(19) United States
(12) Patent Application Publication
(10) Pub. No.: US 2024/0294648 A1
(43) Pub. Date: Sep. 5, 2024

(54) MANAbodies AND METHODS OF USING

(71) Applicant: The Johns Hopkins University, Baltimore, MD (US)

(72) Inventors: Emily Han-Chung Hsiue, Baltimore, MD (US); Qing Wang, Owings Mills, MD (US); Bert Vogelstein, Baltimore, MD (US); Kenneth W. Kinzler, Frankford, DE (US); Shibin Zhou, Owings Mills, MD (US); Jacqueline Douglass, Baltimore, MD (US); Michael S. Hwang, Seattle, WA (US); Nickolas Papadopoulos, Towson, MD (US)

(21) Appl. No.: 18/473,695

(22) Filed: Sep. 25, 2023

Related U.S. Application Data
(63) Continuation of application No. 16/614,005, filed on Nov. 15, 2019, now Pat. No. 11,807,662.
(60) Provisional application No. 62/506,674, filed on May 16, 2017.

(51) Int. Cl. C07K 16/28; A61K 35/17; A61K 38/00; C07K 14/705; C07K 14/725; C07K 14/73

(52) U.S. Cl. CPC C07K 16/2833; A61K 35/17; C07K 14/7051; C07K 14/70514; C07K 14/70517; C07K 14/70521; C07K 16/2809; A61K 38/00; C07K 2317/24; C07K 2317/31; C07K 2317/33; C07K 2317/622; C07K 2319/03

(57) ABSTRACT

This document provides methods and materials for assessing a mammal having or suspected of having cancer and/or for treating a mammal having cancer. For example, molecules including one or more antigen-binding domains (e.g., a single-chain variable fragment (scFv)) that can bind to a modified peptide (e.g., a tumor antigen), as well as method for using such molecules, are provided.

### CROSS-REFERENCE TO RELATED APPLICATIONS

This application is a continuation of U.S. application Ser. No. 16/614,005, filed on Nov. 15, 2019, which is a National Stage application under 35 U.S.C. § 371 of International Application No. PCT/US2018/032996, having an International Filing Date of May 16, 2018, which claims the benefit of U.S. Patent Application Ser. No. 62/506,674, filed on May 16, 2017. The disclosures of the prior applications are considered part of and are incorporated by reference in the disclosure of this application.

### STATEMENT REGARDING FEDERAL FUNDING

This invention was made with government support under grant CA062924 awarded by the National Institutes of Health. The government has certain rights in the invention.

### SEQUENCE LISTING

This application contains a Sequence Listing that has been submitted electronically as an XML file named “44807-0146002_SL.xml.” The XML file, created on Sep. 21, 2023, is 368000 bytes in size. The material in the XML file is hereby incorporated by reference in its entirety.

### BACKGROUND

### 1. Technical Field

This document relates to methods and materials for assessing a mammal having or suspected of having cancer and/or for treating a mammal having cancer. For example, this document provides methods and materials for using a molecule including one or more antigen-binding domains (e.g., a single-chain variable fragment (scFv)) that can bind to a modified peptide (e.g., a tumor antigen) to treat a mammal having a cancer.

### 2. Background Information

Somatic mutations in cancer are ideal targets for cancer therapy as they are uniquely expressed only in tumor cells and not normal cells. In particular, targeting driver gene proteins (broadly subdivided into oncogene proteins and tumor suppressor proteins) have added benefits. First, the tumor's dependence on their oncogenic-endowing capacity makes resistance less likely. Second, these mutations typically occur early during the development of the tumor, thus essentially all daughter cancer cells will contain the mutation. Finally, driver gene proteins tend to have hotspot mutations shared among many patients, thus a therapy targeting a single mutation could be applied to a broad patient population.

Most mutant proteins, including most mutant driver gene proteins, are intracellular. While small molecules can target intracellular proteins, developing small molecules that can specifically inhibit the activity of a mutant driver gene and not its wild-type (wt) counterpart has remained out of reach for the majority of such driver gene proteins. Antibodies, which can have the capacity to distinguish a single amino acid mutation, can typically only target extracellular epitopes.

The immune system samples the intracellular contents of cells through antigen processing and presentation. Following protein proteolysis, a fraction of the resulting peptides are loaded onto human leukocyte antigen (HLA) and sent to the cell surface where they serve as a way for T cells, via their T cell receptor (TCR), to distinguish self from non-self peptides. For example, a virally-infected cell will present viral peptides in its HLA, triggering T cells to kill that cell. Similarly, in cancer, mutant peptides can be presented in HLA on the cancer cell surface, referred to as MANAs, for Mutation-Associated Neo-Antigens. In some cases, and to varying degrees, patients may mount an anti-cancer T cell response against these mutant-peptide-HLA neoantigens, and checkpoint blockade antibodies can further augment this response. However, many patients, particularly those with a low mutational burden, cannot mount a sufficient anti-cancer T cell response. A therapy or diagnostic specifically targeting MANAs could therefore provide a truly tumor-specific method to diagnose or treat cancer.

HLA class I proteins are present on all nucleated cells. There are three classical HLA class I genes, A, B, and C, each of which are highly polymorphic. Each HLA allele has a particular peptide-binding motif, and as a result, only certain peptides will bind to certain HLA alleles.

There is a continuing need in the art to develop new methods to diagnose, monitor, and effectively treat cancers.

### SUMMARY

This document provides methods and materials for treating a mammal having cancer. For example, this document provides methods and materials for using one or more molecules including one or more antigen-binding domains (e.g., scFvs) that can bind to a modified peptide (e.g., a modified peptide present in a peptide-HLA-b2M complex) to treat a mammal having a cancer (e.g., a cancer expressing the modified peptide). In some cases, one or more molecules including one or more antigen-binding domains (e.g., scFvs) that can bind to a modified peptide (e.g., a modified peptide present in a peptide-HLA-b2M complex) can be administered to a mammal having a cancer (e.g., a cancer expressing the modified peptide) to treat the mammal.

As demonstrated herein, scFvs were identified that target (e.g., bind to) numerous Mutation-Associated Neo-Antigens (MANAs) present in a peptide-HLA-b2M complex in many acute myeloid leukemia (AML) cases. Also as demonstrated herein, the scFvs were used to design both chimeric antigen receptor (CAR) T cells (CARTs; also abbreviated as CAR Ts or CAR-Ts) and bispecific antibodies capable of recognizing and killing cells expressing MANAs. The ability to specifically target MANAs provides a tumor-specific method to diagnose and/or treat cancer. For example, scFvs specifically targeting MANAs can be used in full-length antibodies or fragments thereof, antibody drug conjugates (ADCs), antibody radionuclide conjugates, CARTs, or bispecific antibodies to diagnose and/or treat a mammal having cancer.

In general, one aspect of this document a molecule comprising an antigen-binding domain that can bind to a peptide-HLA-beta-2 microglobulin complex, where the peptide includes a modified peptide, where the HLA is a class I HLA, and where the antigen-binding domain does not bind to a complex that includes a wild-type version of the modified peptide. The modified peptide can include from 7 amino acids to 15 amino acids (e.g., 10 amino acids). The modified peptide can be derived from a modified IDH2 polypeptide, a modified EGFR polypeptide, a modified p53 polypeptide, a modified KRAS polypeptide, a modified HRAS polypeptide, a modified NRAS polypeptide, or a modified CTNNB polypeptide. The modified peptide can include an amino acid sequence set forth SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, or SEQ ID NO:32. When the modified peptide includes SEQ ID NO:1, the class I HLA can be an HLA-B7, and the antigen binding fragment can include an amino acid sequence set forth in SEQ ID NO:3, SEQ ID NO:4, SEQ ID NO:5, SEQ ID NO:6, or SEQ ID NO:8. When the modified peptide includes SEQ ID NO: 11, the class I HLA can be an HLA-B7, and the antigen binding fragment can include an amino acid sequence set forth in SEQ ID NO:380, SEQ ID NO:390, SEQ ID NO:391, SEQ ID NO:392, or SEQ ID NO:393. When the modified peptide includes SEQ ID NO: 13, the class I HLA can be an HLA-A2, and the antigen binding fragment can include an amino acid sequence set forth in SEQ ID NO:324, SEQ ID NO:325, SEQ ID NO:326, SEQ ID NO:327, SEQ ID NO:328, SEQ ID NO:329, or SEQ ID NO:330. When the modified peptide includes SEQ ID NO:15, the class I HLA can be an HLA-A2, and the antigen binding fragment can include an amino acid sequence set forth in SEQ ID NO:331, SEQ ID NO:333, SEQ ID NO:336, or SEQ ID NO:337. When the modified peptide includes SEQ ID NO:16, the class I HLA can be an HLA-A2, and the antigen binding fragment can include an amino acid sequence set forth in SEQ ID NO:332, SEQ ID NO:334, SEQ ID NO:335, SEQ ID NO:336, or SEQ ID NO:337. When the modified peptide includes SEQ ID NO: 18, the class I HLA can be an HLA-A2, and the antigen binding fragment can include an amino acid sequence set forth in SEQ ID NO:338, SEQ ID NO:339, or SEQ ID NO:340. When the modified peptide includes SEQ ID NO:20, the class I HLA can be an HLA-A3, and the antigen binding fragment can include an amino acid sequence set forth in SEQ ID NO:341, SEQ ID NO:342, or SEQ ID NO:343. When the modified peptide includes SEQ ID NO:21, the class I HLA can be an HLA-A3, and the antigen binding fragment can include an amino acid sequence set forth in SEQ ID NO:342, SEQ ID NO:343, SEQ ID NO:349, SEQ ID NO:350, SEQ ID NO:351, SEQ ID NO:352, SEQ ID NO:353, SEQ ID NO:354, SEQ ID NO:355, SEQ ID NO:356, or SEQ ID NO:357. When the modified peptide includes SEQ ID NO:22, the class I HLA can be an HLA-A3, and the antigen binding fragment can include an amino acid sequence set forth in SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:369, SEQ ID NO:370, SEQ ID NO:371, SEQ ID NO:372, SEQ ID NO:373, or SEQ ID NO:374. When the modified peptide includes SEQ ID NO:24, the class I HLA can be an HLA-A11, and the antigen binding fragment can include an amino acid sequence set forth in SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, or SEQ ID NO:368. When the modified peptide includes SEQ ID NO:26, the class I HLA can be an HLA-A3, and the antigen binding fragment can include an amino acid sequence set forth in SEQ ID NO:375, SEQ ID NO:376, SEQ ID NO:377, SEQ ID NO:378, or SEQ ID NO:379. When the modified peptide includes SEQ ID NO:28, the class I HLA can be an HLA-A1, and the antigen binding fragment can include an amino acid sequence set forth in SEQ ID NO:394. When the modified peptide includes SEQ ID NO:30, the class I HLA can be an HLA-A1, and the antigen binding fragment can include an amino acid sequence set forth in SEQ ID NO:395. When the modified peptide includes SEQ ID NO:31, the class I HLA can be an HLA-A1, and the antigen binding fragment can include an amino acid sequence set forth in SEQ ID NO:396. When the modified peptide includes SEQ ID NO:32, the class I HLA can be an HLA-A1, and the antigen binding fragment can include an amino acid sequence set forth in SEQ ID NO:397, SEQ ID NO: 398, SEQ ID NO:399, SEQ ID NO:400, or SEQ ID NO:401. The molecule can be an antibody, an antibody fragment, a scFv, a CAR, a TCR, a TCR mimic, a tandem scFv, a bispecific T cell engager, a diabody, a single-chain diabody, an scFv-Fc, a bispecific antibody, or a dual-affinity re-targeting antibody (DART). For example, the molecule can be a single-chain diabody. The molecule also can include an antigen-binding domain that can bind to an effector cell receptor (e.g., CD3, CD28, CD4, CD8, CD16a, NKG2D, PD-1, CTLA-4, 4-1BB, OX40, ICOS, or CD27). When the antigen-binding domain that can bind to an effector cell can bind to CD3, the antigen-binding domain can include an amino acid sequence set forth in SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, or SEQ ID NO:417.

In another aspect, this document features a CAR. The CAR can include an extracellular domain that includes any antigen-binding domain described herein (e.g., an antigen-binding domain that can bind to a peptide-HLA-beta-2 microglobulin complex, where the peptide includes a modified peptide, where the HLA is a class I HLA, and where the antigen-binding domain does not bind to a complex that includes a wild-type version of the modified peptide), a transmembrane domain, and an intracellular domain. The transmembrane domain can include a transmembrane domain of CD4, CD8, or CD28. The intracellular domain can include one or more costimulatory domains from CD28, DAP10, ICOS, OX40, and/or 4-1BB. The intracellular domain can include a signaling domain from CD3-zeta.

In another aspect, this document features a T cell expressing any CAR described herein (e.g., a CAR including an extracellular domain that includes any antigen-binding domain described herein, a transmembrane domain, and an intracellular domain). The T cell can express a CAR including an extracellular domain that includes an antigen-binding domain that can bind to a peptide-HLA-beta-2 microglobulin complex, where the peptide includes a modified peptide, where the HLA is a class I HLA, and where the antigen-binding domain does not bind to a complex that includes a wild-type version of the modified peptide), a transmembrane domain, and an intracellular domain.

In another aspect, this document features methods for treating a mammal having a cancer. The methods can include, or consist essentially of, administering to a mammal one or more molecules that include any antigen-binding domain described herein (e.g., an antigen-binding domain that can bind to a peptide-HLA-beta-2 microglobulin complex, where the peptide includes a modified peptide, where the HLA is a class I HLA, and where the antigen-binding domain does not bind to a complex that includes a wild-type version of the modified peptide), wherein the cancer includes cancer cells expressing a modified peptide. The mammal can be a human. The cancer can be Hodgkin's lymphoma, non-Hodgkin's lymphoma, AML, a lung cancer, a pancreatic cancer, a gastric cancer, a colorectal cancer, an ovarian cancer, an endometrial cancer, a biliary tract cancer, a liver cancer, myeloma, a breast cancer, a prostate cancer, an esophageal cancer, a stomach cancer, a kidney cancer, a bone cancer, a soft tissue cancer, a head and neck cancer, a glioblastoma multiforme, or an astrocytoma.

In another aspect, this document features methods for treating a mammal having a cancer. The methods can include, or consist essentially of, administering to a mammal one or more T cells expressing any one of the CARs described herein (e.g., a CAR including an extracellular domain that includes any antigen-binding domain described herein, a transmembrane domain, and an intracellular domain), where the cancer includes cancer cells expressing a modified peptide. The mammal can be a human. The cancer can be Hodgkin's lymphoma, non-Hodgkin's lymphoma, AML, a lung cancer, a pancreatic cancer, a gastric cancer, a colorectal cancer, an ovarian cancer, an endometrial cancer, a biliary tract cancer, a liver cancer, myeloma, a breast cancer, a prostate cancer, an esophageal cancer, a stomach cancer, a kidney cancer, a bone cancer, a soft tissue cancer, a head and neck cancer, a glioblastoma multiforme, or an astrocytoma.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains ELISA results showing specificity of IDH2 R140Q HLA-B7 scFvs. Peptide-HLA biotinylated monomers were coated on a streptavidin plate, including the wild type (wt) version of an IDH2 peptide (SPNGTIRNIL; SEQ ID NO:2), an IDH2 peptide containing the R140Q mutation (SPNGTIQNIL; SEQ ID NO:1), and four control HLA-B7 monomers containing the following control peptides: control 1 peptide is SPGAANKRPI (an artificial sequence; SEQ ID NO:418), control 2 peptide is RPIPIKYKAM (from mutant MyD88 L265P; SEQ ID NO:9), control 3 peptide is KPITIGRHAH (from a different peptide from wt IDH2; SEQ ID NO:10), and control 4 peptide is AVGVGKSAL (from mutant KRAS G12V; SEQ ID NO:11). The five clones identified in panning were incubated in the wells at the specified dilutions, followed by a rabbit anti-phage antibody, then anti-Rabbit-HRP antibody.

FIG. 2 contains a graph showing flow cytometry on peptide-pulsed A2+ cells. T2 cells were peptide-pulsed overnight at 37° C. in serum-free media with beta-2 microglobulin (b2M) protein only, or b2M with a EGFR T790M(789-797) peptide (IMQLMPFGC; SEQ ID NO:13). The EGFR wt(789-797) peptide (ITQLMPFGC; SEQ ID NO:14) did not bind to HLA-A2. Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 3 contains a graph showing flow cytometry on peptide-pulsed B7+ cells. RPMI-6666 cells were peptide pulsed overnight at 37° C. in serum-free media with b2M protein only, b2M protein with an IDH2 mutant R140Q peptide (SPNGTIQNIL; SEQ ID NO:1), or b2M with the IDH2 wt peptide (SPNGTIRNIL; SEQ ID NO:2). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 4 contains a graph showing flow cytometry on peptide-pulsed A2+ cells. T2 cells were peptide-pulsed overnight at 37° C. in serum-free media with b2M only, b2M with a p53 mutant R248Q(245-254) peptide (GMNQRPILTI; SEQ ID NO:15), b2M with a p53 mutant R248W(245-254) peptide (GMNWRPILTI; SEQ ID NO:16), b2M with the p53 wt(245-254) peptide (GMNRRPILTI; SEQ ID NO:17), or b2M with an HLA-A2 control peptide ELA (ELAGIGILTV; SEQ ID NO:403). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 5 contains a graph showing flow cytometry on peptide-pulsed A2+ cells. T2 cells were peptide-pulsed overnight at 37° C. in serum-free media with b2M only, b2M with a KRAS mutant G12V(6-14) peptide (LVVVGAVGV; SEQ ID NO:18), or b2M with the KRAS wt(6-14) peptide (LVVVGAGGV; SEQ ID NO:19). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 6 contains a graph showing flow cytometry on peptide-pulsed A3+ cells. T2A3 cells were peptide-pulsed overnight at 37° C. in serum-free media with b2M only, b2M with a KRAS mutant G12C(8-16) peptide (VVGACGVGK; SEQ ID NO:419), b2M with a KRAS mutant G12D(8-16) peptide (VVGADGVGK; SEQ ID NO:420), b2M with a KRAS mutant G12V(8-16) peptide (VVGAVGVGK; SEQ ID NO:22), b2M with a KRAS mutant G12C(7-16) peptide (VVVGACGVGK; SEQ ID NO:20), b2M with a KRAS mutant G12D(7-16) peptide (VVVGADGVGK; SEQ ID NO:21), b2M with a KRAS mutant G12V(7-16) peptide (VVVGAVGVGK; SEQ ID NO:22), b2M with the KRAS wt(8-16) peptide (VVGAGGVGK; SEQ ID NO:25), b2M with the KRAS wt(7-16) peptide (VVVGAGGVGK; SEQ ID NO:23), or the CTNNB S45F(41-49) peptide (TTAPFLSGK; SEQ ID NO:26). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 7 contains a graph showing flow cytometry on peptide-pulsed A3+ cells. T2A3 cells were peptide-pulsed overnight at 37° C. in serum-free media with b2M only, b2M with a KRAS mutant G12V(7-16) peptide (VVVGAVGVGK; SEQ ID NO:22), or b2M with the KRAS wt(7-16) peptide (VVVGAGGVGK; SEQ ID NO:23). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 8 contains a graph showing flow cytometry on peptide-pulsed A3+ cells. T2A3 cells were peptide-pulsed overnight at 37° C. in serum-free media with b2M only, b2M with a KRAS mutant G12C(7-16) peptide (VVVGACGVGK; SEQ ID NO:20), b2M with a KRAS mutant G12D(7-16) peptide (VVVGADGVGK; SEQ ID NO:21), b2M with a KRAS mutant G12V(7-16) peptide (VVVGAVGVGK; SEQ ID NO:22), or b2M with the KRAS wt(7-16) peptide (VVVGAGGVGK; SEQ ID NO:23). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 9 contains a graph showing flow cytometry on peptide-pulsed A11+ cells. Hs611.T cells were peptide-pulsed overnight at 37° C. in serum-free media with b2M only, b2M with a KRAS mutant G12C(7-16) peptide (VVVGACGVGK; SEQ ID NO:20), b2M with a KRAS mutant G12D(7-16) peptide (VVVGADGVGK; SEQ ID NO:21), b2M with a KRAS mutant G12V(7-16) peptide (VVVGAVGVGK; SEQ ID NO:22), or b2M with the KRAS wt(7-16) peptide (VVVGAGGVGK; SEQ ID NO:23). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 10 contains a graph showing flow cytometry on peptide-pulsed A11+ cells. MINO cells were peptide-pulsed overnight at 37° C. in serum-free media with b2M only, b2M with a KRAS mutant G12D(8-16) peptide (VVGADGVGK; SEQ ID NO:24), or b2M with the KRAS wt(8-16) peptide (VVGAGGVGK; SEQ ID NO:25). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 11 contains a graph showing flow cytometry on peptide-pulsed A11+ cells. Hs611.T cells were peptide-pulsed overnight at 37° C. in serum-free media with b2M only, b2M with a KRAS mutant G12C(7-16) peptide (VVVGACGVGK; SEQ ID NO:20), b2M with a KRAS mutant G12D(7-16) peptide (VVVGADGVGK; SEQ ID NO:21), b2M with a KRAS G12V(7-16) peptide (VVVGAVGVGK; SEQ ID NO:22), or b2M with the KRAS wt(7-16) peptide (VVVGAGGVGK; SEQ ID NO:23). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 12 contains a graph showing flow cytometry on peptide-pulsed A3+ cells. T2A3 cells were peptide-pulsed overnight at 37° C. in serum-free media with b2M only, b2M with a CTNNB mutant S45F(41-49) peptide (TTAPFLSGK; SEQ ID NO:26), or b2M with CTNNB wt(41-49) peptide (TTAPSLSGK; SEQ ID NO:27). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 13 contains a graph showing flow cytometry on peptide-pulsed B7+ cells. RPMI-6666 cells were peptide-pulsed overnight at 37° C. in serum-free media shows cells pulsed with b2M only, b2M with a KRAS mutant G12V(11-19) peptide (AVGVGKSAL; SEQ ID NO:11). The KRAS wt(11-19) peptide (AGGVGKSAL; SEQ ID NO:12) did not bind to HLA-B7. Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIG. 14 contains a graph showing flow cytometry on peptide-pulsed A1+ cells. SigM5 cells were peptide-pulsed overnight at 37° C. in serum-free media shows cells pulsed with b2M only, b2M with a H/K/N RAS mutant Q61H(55-64) peptide (ILDTAGHEEY; SEQ ID NO:28), b2M with a H/K/N RAS mutant Q61K(55-64) peptide (ILDTAGKEEY; SEQ ID NO:30), b2M with a H/K/N RAS mutant Q61L(55-64) peptide (ILDTAGLEEY; SEQ ID NO:31), b2M with a H/K/N RAS mutant Q61R(55-64) peptide (ILDTAGREEY; SEQ ID NO:32), or b2M with the H/K/N RAS wt(55-64) peptide (ILDTAGQEEY; SEQ ID NO:29). Cells were stained with 10 μL of precipitated phage per 100 μL of cells, washed and stained with rabbit anti-M13 antibody, then washed and stained with anti-Rabbit-PE antibody. Cells were stained with a live/dead Near-IR dye, washed, and analyzed by an LSRII flow cytometer.

FIGS. 15A-15B show that MANAbody clones can be converted into CAR-T cells. FIG. 15A: IDH2 R140Q(134-143)-B7 cl.1 MANA-CAR-T cells were co-cultured for 4 hours at 37° C. with COS-7 cells co-transfected with plasmids encoding various combinations of HLA-B7, IDH2(WT), and IDH2(R140Q). Following co-culture, conditioned media was collected and assayed for secreted IFNγ by ELISA. FIG. 15B: KRAS G12V(7-16)-A3 cl.2 MANA-CAR-T cells were co-cultured for 4 hours at 37° C. with COS-7 cells transfected with plasmids encoding various combinations of HLA-A3, KRAS(WT), and KRAS(G12V). Following co-culture, conditioned media was collected and assayed for secreted IFNγ by ELISA.

FIGS. 16A-16B show that MANAbody clones can be converted into single-chain diabodies (scDbs). FIG. 16A: IDH2 R140Q(134-143)-B7 cl.29, cl. 1, and cl.3 scDbs, containing either the anti-CD3 clone mUCHT1 or hUCHT1v9, were incubated at the specified concentrations with T cells and COS-7 cells co-transfected with plasmids encoding various combinations of HLA-B7, IDH2(WT), IDH2(R140Q), and GFP for 24 hours at 37° C. Following co-culture, plates was snap frozen and conditioned media was collected and assayed for secreted IFNγ by ELISA. FIG. 16B: KRAS G12V(7-16)-A3 cl.2 mUCHT1 scDb was incubated at the specified concentrations with or without T cells and either 1) no target cells, 2) COS-7 cells co-transfected with plasmids encoding HLA-A3 and KRAS(WT) or HLA-A3 and KRAS(G12V), or 3) with NCI-H441 parental or HLA-A3 knockout cells for 24 hours at 37° C. Following co-culture, plates was snap frozen and conditioned media was collected and assayed for secreted IFNγ by ELISA.

FIG. 17 shows that a MANAbody clone converted into a scDb can kill target cells. KRAS G12V(7-16)-A3 cl.2 mUCHT1 scDb and a pan-HLA-A3 scDb were incubated at 0 or 50 ng/mL with or without T cells and with NCI-H441 parental or HLA-A3 knockout cells for 24 hours at 37° C. Following co-culture, CellTiter-Glo® was used to assay viable cells in each well. Percent target cell viability was calculated by subtracting the value from T cell only wells and normalizing to the value from target cell only wells.

### DETAILED DESCRIPTION

This document provides methods and materials for assessing a mammal having cancer or suspected of having cancer and/or treating a mammal having cancer. For example, one or more molecules including one or more antigen-binding domains (e.g., scFvs) that can target (e.g., bind to) one or more modified peptides (e.g., peptides present in a peptide-HLA complex such as a peptide-HLA-b2M complex) can be used to assess a mammal having cancer or suspected of having cancer and/or to treat a mammal having a cancer (e.g., a cancer expressing one or more modified peptides). In some cases, one or more molecules includes one or more antigen-binding domains that can bind to a modified peptide can be used to detect the presence or absence of one or more modified peptides in a sample obtained from a mammal having cancer or suspected of having cancer. In some cases, one or more molecules including one or more antigen-binding domains that can bind to a modified peptide can be administered to a mammal having a cancer (e.g., a cancer expressing the modified peptide) to treat the mammal.

As used herein, a modified peptide is a peptide derived from a modified polypeptide. A modified polypeptide can be any appropriate modified polypeptide (e.g., a polypeptide having a disease causing mutation such as an oncogenic mutation). A modified peptide can have one or more amino acid modifications (e.g., substitutions) relative to a wild type (wt) peptide (e.g., a peptide derived from a wt polypeptide from which the modified polypeptide is derived). A modified peptide also can be referred to as a mutant peptide. In some cases, a modified peptide can be a tumor antigen. Examples of tumor antigens include, without limitation, mutation-associated neo-antigens (MANAs), tumor-associated antigen, and tumor-specific antigens. A modified peptide can be any appropriate length. In some cases, a modified peptide can be from about 7 amino acids to about 15 amino acids (e.g., from about 8 amino acids to about 15 amino acids, from about 9 amino acids to about 15 amino acids, from about 10 amino acids to about 15 amino acids, from about 11 amino acids to about 15 amino acids, from about 12 amino acids to about 15 amino acids, from about 13 amino acids to about 15 amino acids, from about 7 amino acids to about 14 amino acids, from about 7 amino acids to about 13 amino acids, from about 7 amino acids to about 12 amino acids, from about 7 amino acids to about 11 amino acids, from about 7 amino acids to about 10 amino acids, from about 7 amino acids to about 9 amino acids, or from about 9 amino acids to about 10 amino acids) in length. For example, a modified peptide can be about 9 amino acids in length. For example, a modified peptide can be about 10 amino acids in length. A modified peptide can be derived from any modified (e.g., oncogenic) polypeptide. Examples of modified polypeptides from which modified peptides described herein can be derived include, without limitation, epidermal growth factor receptor (EGFR), isocitrate dehydrogenase 2 (IDH2), p53, RAS (e.g., KRAS, HRAS, and NRAS), and CTNNB. A modified peptide can include any appropriate modification. In some cases, modified peptides described herein can include one or more modifications (e.g., mutations) shown in Table 1.

TABLE 1
Modified peptides.

| Protein | | | SEQ | | SEQ | Peptide | HLA |
|---|---|---|---|---|---|---|---|
| of origin | Mutation | Mutant Peptide(s) | ID NO: | WT Peptide | ID NO: | Codons | Allele |
| EGFR | T790M | IMQLMPFGC | 13 | ITQLMPFGC | 14 | 789-797 | A2 |
| IDH2 | R140Q | SPNGTIQNIL | 1 | SPNGTIRNIL | 2 | 134-143 | B7 |
| p53 | R248Q, | GMNQRPILTI, | 15 | GMNRRPILTI | 17 | 245-254 | A2 |
| | R248W | GMNWRPILTI | 16 | | | | |
| KRAS | G12V | LVVVGAVGV | 18 | LVVVGAGGV | 19 | 6-14 | A2 |
| KRAS | G12C, | VVVGACGVGK, | 20 | VVVGAGGVGK | 23 | 7-16 | A3 |
| | G12D, | VVVGADGVGK, | 21 | | | | |
| | G12V | VVVGAVGVGK | 22 | | | | |
| KRAS | G12V | VVVGAVGVGK | 22 | VVVGAGGVGK | 23 | 7-16 | A3 |
| KRAS | G12D | VVVGADGVGK | 21 | VVVGAGGVGK | 23 | 7-16 | A3 |
| KRAS | G12D | VVVGADGVGK | 21 | VVVGAGGVGK | 23 | 7-16 | A11 |
| KRAS | G12D | VVGADGVGK | 24 | VVGAGGVGK | 25 | 8-16 | A11 |
| KRAS | G12V | VVVGAVGVGK | 22 | VVVGAGGVGK | 23 | 7-16 | A11 |
| CTNNB | S45F | TTAPFLSGK | 26 | TTAPSLSGK | 27 | 41-49 | A3 |
| KRAS | G12V | AVGVGKSAL | 11 | AGGVGKSAL | 12 | 11-19 | B7 |
| H/K/N RAS | Q61H | ILDTAGHEEY | 28 | ILDTAGQEEY | 29 | 55-64 | A1 |
| H/K/N RAS | Q61K | ILDTAGKEEY | 30 | ILDTAGQEEY | 29 | 55-64 | A1 |
| H/K/N RAS | Q61L | ILDTAGLEEY | 31 | ILDTAGQEEY | 29 | 55-64 | A1 |
| H/K/N RAS | Q61R | ILDTAGREEY | 32 | ILDTAGQEEY | 29 | 55-64 | A1 |

A modified peptide described herein (e.g., a modified peptide including an amino acid sequence set forth in any one of SEQ ID NO:1, SEQ ID NO:11, SEQ ID NO:13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be in a complex with any appropriate HLA. An HLA can be any appropriate HLA allele. In some cases, an HLA can be a class I HLA (e.g., HLA-A, HLA-B, and HLA-C) allele. Examples of HLA alleles that a modified peptide described herein can complex with include, without limitation, HLA-A1, HLA-A2, HLA-A3, HLA-11, and HLA-B7. Exemplary HLA alleles for particular modified peptides are shown in Table 1. For example, a modified peptide derived from a modified EGFR polypeptide (e.g., IMQLMPFGC (SEQ ID NO:13)) can be in a complex with HLA-A2 and b2M. For example a modified peptide derived from a modified IDH2 polypeptide (e.g., SPNGTIQNIL (SEQ ID NO:1)) can be in a complex with HLA-B7 and b2M. For example a modified peptide derived from a modified p53 polypeptide (e.g., GMNQRPILTI (SEQ ID NO:15) or GMNWRPILTI 1(SEQ ID NO:16)) can be in a complex with HLA-A2 and b2M. For example a modified peptide derived from a modified KRAS polypeptide (e.g., LVVVGAVGV (SEQ ID NO:18), VVVGACGVGK (SEQ ID NO:20), VVVGADGVGK (SEQ ID NO:21), VVVGAVGVGK (SEQ ID NO:22), and VVGADGVGK (SEQ ID NO:24)) can be in a complex with HLA-A2, HLA-A3, and/or HLA-A11, and b2M. For example a modified peptide derived from a modified CTNNB polypeptide (e.g., TTAPFLSGK (SEQ ID NO:26)) can be in a complex with HLA-A3 and b2M. For example a modified peptide derived from a modified KRAS polypeptide (e.g., AVGVGKSAL (SEQ ID NO:11)) can be in a complex with HLA-B7 and b2M. For example a modified peptide derived from a modified H/K/N RAS polypeptide (e.g., ILDTAGHEEY (SEQ ID NO:28), ILDTAGKEEY (SEQ ID NO:30), ILDTAGLEEY (SEQ ID NO:31), ILDTAGREEY (SEQ ID NO:32)) can be in a complex with HLA-A1 and b2M.

This document provides molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO: 15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32). In some cases, a molecule including one or more antigen-binding domains that can bind to a modified peptide described herein does not target (e.g., does not bind to) a modified peptide described herein that is not present in a complex (e.g., a peptide-HLA-b2M complex). In some cases, a molecule including one or more antigen-binding domains that can bind to a modified peptide described herein does not target (e.g., does not bind to) a wt peptide (e.g., a peptide derived from a wt polypeptide from which the modified polypeptide is derived).

A molecule including one or more antigen-binding domains (e.g., scFvs) that can bind to a modified peptide described herein can be any appropriate type of molecule. In some cases, a molecule can be a monovalent molecule (e.g., containing a single antigen-binding domain). In some cases, a molecule can be a multivalent molecule (e.g., containing two or more antigen-binding domains and simultaneously targeting two or more antigens). For example, a bispecific molecule can include two antigen-binding domains, a trispecific molecule can include three antigen-binding domains, a quadspecific molecule can include four antigen-binding domains, etc. Examples of molecules that contain antigen-binding domains include, without limitation, antibodies, antibody fragments, scFvs, CARs, T cell receptors (TCRs), TCR mimics, tandem scFvs, bispecific T cell engagers, diabodies, scDbs, scFv-Fcs, bispecific antibodies, dual-affinity re-targeting antibodies (DARTs), and any other molecule that includes at least one variable heavy chain (VH) and at least one variable light chain (VL). Any of these molecules can be used in accordance with materials and methods described herein. In some cases, an antigen-binding domain can be a scFv. For example, a molecule including one or more antigen-binding domains (e.g., one or more scFvs) that can bind to a modified peptide described herein can be a CAR. For example, a molecule including two scFvs that can bind to a modified peptide described herein can be a single-chain diabody (scDb).

In some cases, when a molecule including one or more antigen-binding domains (e.g., one or more scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) is a CAR, the CAR can be any appropriate CAR. A CAR provided herein can include an extracellular domain having at least one antigen-binding domain that can bind to a modified peptide described herein, a transmembrane domain, and an intracellular domain (e.g., an intracellular signaling domain such as a costimulatory domain). A CAR can include any appropriate extracellular domain. For example, a CAR can include a molecule (e.g., a scFv) having an antigen binding domain that can bind to a modified peptide including an amino acid sequence set forth in any one of SEQ ID NO:1, SEQ ID NO: 11, SEQ ID NO: 13, SEQ ID NO: 15, SEQ ID NO: 16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32. A CAR can include any appropriate transmembrane domain. A transmembrane domain can be derived from any appropriate polypeptide. Examples of transmembrane domains that can be used in CAR described herein include, without limitation, transmembrane domains of CD4, CD8 (e.g., CD8-alpha and CD8-beta), CD28, CD3 epsilon, CD5, CD6, CD9, CD16, CD22, CD33, CD37, CD45, CD64, CD80, CD86, CD134, 4-1BB, and CD154. In some cases, a CAR described herein can include a CD28 transmembrane domain. A CAR can include any appropriate intracellular domain. An intracellular domain can be derived from any appropriate polypeptide. An intracellular domain can include a costimulatory domain (e.g., a single costimulatory domain or multiple costimulatory domains). In cases where a CAR includes multiple costimulatory domains, the CAR can include multiple costimulatory domains of the same type or multiple costimulatory domains of different types. An intracellular domain can include a signaling domain. Examples of intracellular domains that can be used in CAR described herein include, without limitation, intracellular domains of CD3 (e.g., a CD3-zeta), CD28, DAP10, inducible T-cell costimulator (ICOS), OX40, 4-1BB, CD2, CD4, CD8, CD5, CD22, DAP-12, CD22, and CD79. A CAR can be made using any appropriate method. In some cases, a CAR also can include a hinge sequence (e.g., positioned between the extracellular domain and the transmembrane domain). In some cases, a CAR can be made as described elsewhere (see, Curran et al., 2012 J. Gene Med 14:405-415; Kershaw et al., 2005 Nature Reviews Immunol. 5(12):928-940; Eshhar et al., 1993 Proc. Natl. Acad. Sci. U.S.A. 90(2):720-724; Sadelain et al., 2009 Curr. Opin. Immunol. 21(2): 215-223; WO 2015/142675; WO 2015/150526; and WO 2014/134165). Also provided here are CARTs expressing one or more CARs, which CARs can target (e.g., bind to) one or more modified peptides described herein (e.g., CARs having two or more antigen-binding domains). Also provided here are CARTs expressing one or more CARs, which CARTs can target (e.g., bind to) one or more modified peptides described herein.

In some cases, when a molecule including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO: 15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) is a multivalent molecule (e.g., a bispecific molecule), a first antigen-binding domain can bind to a modified peptide described herein and a second antigen-binding domain can bind to an effector cell (e.g., an antigen present on an effector cell). Examples of effector cells include, without limitation, T cells, natural killer (NK) cells, natural killer T (NKT) cells, B cells, plasma cells, macrophages, monocytes, microglia, dendritic cells, neutrophils, fibroblasts, and mast cells. Examples of antigens present on effector cells include, without limitation, CD3, CD4, CD8, CD28, CD16a, NKG2D, PD-1, CTLA-4, 4-1BB, OX40, ICOS, CD27, and any other effector cell surface receptors. In some cases, a molecule described herein can include a first antigen-binding domain that can bind to a modified peptide described herein and a second antigen-binding domain that can bind to an antigen present on a T cell (e.g., CD3). In some cases, sequences (e.g., scFv sequences) that can bind to CD3 can be as shown in Table 4. In some cases, sequences (e.g., scFv sequences) that can bind to CD3 can be as described elsewhere (see, e.g., Rodrigues et al., 1992 Int J Cancer Suppl. 7:45-50; Shalaby et al., 1992 J Exp Med. 175:217-25; Brischwein et al., 2006 Mol Immunol. 43:1129-43; Li et al., 2005 Immunology. 116:487-98; WO2012162067; US20070065437; US20070065437; US20070065437; US20070065437; US20070065437; and US20070065437). In some cases, a molecule described herein can include a first antigen-binding domain that can bind to a modified peptide described herein and a second antigen-binding domain that can bind to an antigen present on a NK cell (e.g., CD16a or NKG2D). By binding both the modified peptide and the effector cell, the multivalent molecule can bring the cell expressing the modified peptide (e.g., as part of the HLA complex) into proximity with the effector cell, permitting the effector cell to act on the cell expressing the modified peptide.

In some cases, when a molecule including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) is a multivalent molecule (e.g., a bispecific molecule), a molecule can be in any appropriate format which includes at least one VH and at least one VL. For example, a VH and a VL can be in any appropriate orientation. In some cases, a VH can be N-terminal to the VL. In some cases, a VH can be C-terminal to the VL. In some cases, a linker amino acid sequence can be positioned between the VH and VL.

In some cases, when a bispecific molecule is a tandem scFv, the tandem scFv can be in any appropriate orientation. Examples of tandem scFv orientations including scFv-A and scFv-B include, without limitation, VLA-LL-VHA-SL-VLB-LL-VHB, VLA-LL-VHA-SL-VHB-LL-VLB, VHA-LL-VLA-SL-VLB-LL-VHB, VHA-LL-VLA-SL-VHB-LL-VLB, VLB-LL-VHB-SL-VLA-LL-VHA, VLB-LL-VHB-SL-VHA-LL-VLA, VHB-LL-VLB-SL-VLA-LL-VHA, and VHB-LL-VLB-SL-VHA-LL-VLA, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination.

In some cases, when a bispecific molecule is a diabody, the diabody can be in any appropriate orientation. Examples of diabody orientations including scFv-A and scFv-B include, without limitation, VLA-SL-VHB and VLB-SL-VHA, VLA-SL-VLB and VHB-SL-VHA, VHA-SL-VLB and VHB-SL-VLA, VLB-SL-VHA and VLA-SL-VHB, VLB-SL-VLA and VHA-SL-VHB, and VHB-SL-VLA and VHA-SL-VLB, where SL is a short linker. A short linker can be from about 3 amino acids to about 10 amino acids in length. A short linker can include any appropriate amino acids (e.g., glycines and serines) in any appropriate combination.

In some cases, when a bispecific molecule is a scDb, the scDb can be in any appropriate orientation. Examples of scDb orientations including scFv-A and scFv-B include, without limitation, VLA-SL-VHB-LL-VLB-SL-VHA, VHA-SL-VLB-LL-VHB-SL-VLA, VLA-SL-VLB-LL-VHB-SL-VHA, VHA-SL-VHB-LL-VLB-SL-VLA, VLB-SL-VHA-LL-VLA-SL-VHB, VHB-SL-VLA-LL-VHA-SL-VLB, VLB-SL-VLA-LL-VHA-SL-VHB, and VHB-SL-VHA-LL-VLA-SL-VLB, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination.

In some cases, when a bispecific molecule is a scFv-Fc, the scFv-Fc can be in any appropriate orientation. Examples of scFv-Fc orientations including scFv-Fc-A, scFv-Fc-B, and an Fc domain include, without limitation, VLA-LL-VHA-hinge-Fc and VLB-LL-VHB-hinge-Fc, VHA-LL-VLA-hinge-Fc and VHB-LL-VLB-hinge-Fc, VLA-LL-VHA-hinge-Fc and VHB-LL-VLB-hinge-Fc, VHA-LL-VLA-hinge-Fc and VLB-LL-VHB-hinge-Fc, where LL is a long linker. A long linker can be from about 10 amino acids to about 25 amino acids in length. A long linker can include any appropriate amino acids (e.g., glycines and serines) in any appropriate combination. In some cases, an Fc domain in a scFv-Fc can include one or more modifications to increase heterodimerization and/or to decrease homodimerization of the scFv-Fc. In some cases, an Fc domain in a scFv-Fc can exclude a hinge domain. In some cases, an Fc domain in a scFv-Fc can be at the N-terminus of the scFv.

A molecule including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can include any appropriate complementarity determining regions (CDRs). For example, a molecule including one or more antigen-binding domains that can bind to a modified peptide described herein can include a variable heavy chain (VH) having three VH complementarity determining regions (CDR-VHs) and a variable light chain (VL) having three VL CDRs (CDR-VLs). For example, a molecule that can bind to a modified peptide derived from a modified EGFR polypeptide (e.g., IMQLMPFGC (SEQ ID NO:13)) can include one of each of the CDRs set forth below:

CDR-VL1:
(SEQ ID NO: 33)
QDVNTA;
CDR-VL2:
SAS;
CDR-VL3:
(SEQ ID NO: 34)
QQYDYAPIT,
(SEQ ID NO: 35)
QQSPYYYLPIT,
(SEQ ID NO: 36)
QQYYYSPVT,
(SEQ ID NO: 37)
QQHYGNPFT,
(SEQ ID NO: 38)
QQSYYSPPT,
(SEQ ID NO: 39)
QQYYSYPPT,
(SEQ ID NO: 40)
QQYYYYPPT;
CDR-VH1:
(SEQ ID NO: 41)
GFNISWYQ,
(SEQ ID NO: 42)
GFNVSWSY,
(SEQ ID NO: 43)
GFNISWNQ,
(SEQ ID NO: 44)
GFNVGYYG,
(SEQ ID NO: 45)
GFNITSSY,
(SEQ ID NO: 46)
GFNINSSY,
(SEQ ID NO: 47)
GFNISTSY;
CDR-VH2:
(SEQ ID NO: 48)
VTPYSGYT,
(SEQ ID NO: 49)
IYGDSGYT,
(SEQ ID NO: 50)
VSPYSGYT,
(SEQ ID NO: 51)
VSGMEGYT,
(SEQ ID NO: 52)
ISPADGYN,
(SEQ ID NO: 53)
ISPTDGYY,
(SEQ ID NO: 54)
IDPNDGYS;
and
CDR-VH3:
(SEQ ID NO: 55)
SRSYTDGFDY,
(SEQ ID NO: 56)
SRGQWEASYYAMDY,
(SEQ ID NO: 57)
SRSDYYAMDY,
(SEQ ID NO: 58)
SRDIYGYAMDV,
(SEQ ID NO: 59)
SRTDSTAYTAMDV,
(SEQ ID NO: 60)
SRTSDTSYAAMDV,
(SEQ ID NO: 61)
SRTNNTAADAMDV.

For example, a molecule that can bind to a modified peptide derived from a modified IDH2 polypeptide (e.g., SPNGTIQNIL (SEQ ID NO:1) can include one of each of the CDRs set forth below:

CDR-VL1:
(SEQ ID NO: 33)
QDVNTA;
CDR-VL2:
SAS;
CDR-VL3:
(SEQ ID NO: 62)
QQYSYSPPT,
(SEQ ID NO: 63)
QQGKAYWPAT,
(SEQ ID NO: 64)
QQVYSSPFT,
(SEQ ID NO: 65)
QQYSLYSPMT,
(SEQ ID NO: 66)
QQSYYMPFT;
CDR-VH1:
(SEQ ID NO: 67)
GFNISDTY,
(SEQ ID NO: 68)
GFNVGHYR,
(SEQ ID NO: 69)
GFNVKYYM,
(SEQ ID NO: 70)
GFNSFLS,
(SEQ ID NO: 71)
GFNIFRGY;
CDR-VH2:
(SEQ ID NO: 72)
ISPRTGYN,
(SEQ ID NO: 73)
VSPNGYYT,
(SEQ ID NO: 74)
ISPGYDYT,
(SEQ ID NO: 75)
IFPSSDYT,
(SEQ ID NO: 76)
ISPHSDYT;
and
CDR-VH3:
(SEQ ID NO: 77)
SRAYYSYAYAMDV,
(SEQ ID NO: 78)
SRGYSSYAFDY,
(SEQ ID NO: 79)
SRSYWRYSVDV,
(SEQ ID NO: 80)
SRGKHSSDSNYYMDY,
(SEQ ID NO: 81)
SRSYGWAAFDY.

For example, a molecule that can bind to a modified peptide derived from a modified p53 polypeptide (e.g., GMNQRPILTI (SEQ ID NO:15) and GMNWRPILTI (SEQ ID NO:16)) can include one of each of the CDRs set forth below:

CDR-VL1:
(SEQ ID NO: 33)
QDVNTA;
CDR-VL2:
SAS;
CDR-VL3:
(SEQ ID NO: 82)
QQSGYAPIT,
(SEQ ID NO: 83)
QQYSYAPIT,
(SEQ ID NO: 84)
QQSLYGPFT,
(SEQ ID NO: 85)
QQYSYSPIT,
(SEQ ID NO: 86)
QQSGYQPDT,
(SEQ ID NO: 87)
QQYLYQPWT;
CDR-VH1:
(SEQ ID NO: 89)
GFNISYYS,
(SEQ ID NO: 90)
GFNIGYYT,
(SEQ ID NO: 91)
GFNIAYEY,
(SEQ ID NO: 92)
GFNLFGYG,
(SEQ ID NO: 93)
GFNISWYA,
(SEQ ID NO: 94)
GFNIDYYG;
CDR-VH2:
(SEQ ID NO: 96)
VDPDSDYT,
(SEQ ID NO: 97)
VSPWSYST,
(SEQ ID NO: 98)
IGPDSGYT,
(SEQ ID NO: 99)
IGPYYYYT,
(SEQ ID NO: 100)
IWPDSDWT,
(SEQ ID NO: 101)
LYGGSDST;
and
CDR-VH3:
(SEQ ID NO: 103)
SRSWIHMFSMDY,
(SEQ ID NO: 104)
SRDHWDEAFDV,
(SEQ ID NO: 105)
SRVWYYSTYGMDY,
(SEQ ID NO: 106)
SRENYDMAMDY,
(SEQ ID NO: 107)
SRYYYSSAFDV,
(SEQ ID NO: 108)
SRQYSAYFDY.

For example, a molecule that can bind to a modified peptide derived from a modified KRAS polypeptide (e.g., LVVVGAVGV (SEQ ID NO:18), VVVGACGVGK (SEQ ID NO:20), VVVGADGVGK (SEQ ID NO:21), VVVGAVGVGK (SEQ ID NO:22), and VVGADGVGK (SEQ ID NO:24)) can include one of each of the CDRs set forth below:

CDR-VL1:
(SEQ ID NO: 33)
QDVNTA;
CDR-VL2:
SAS and SAY;
CDR-VL3:
(SEQ ID NO: 110)
QQWYSSPVT,
(SEQ ID NO: 111)
QQYYSRPVT,
(SEQ ID NO: 121)
QQSYGSGSPWT,
(SEQ ID NO: 122)
QQTYYSPWT,
(SEQ ID NO: 123)
QQYYYPPIT,
(SEQ ID NO: 132)
QQSYYYFRPIT,
(SEQ ID NO: 133)
QQASYYYPLT,
(SEQ ID NO: 134)
QQKSEYSPWT,
(SEQ ID NO: 135)
QQSGYIPFT,
(SEQ ID NO: 136)
QQGAYYRPFT,
(SEQ ID NO: 152)
QQYMYSPVT,
(SEQ ID NO: 153)
QQSSSSPIT,
(SEQ ID NO: 154)
QQSSASPLT,
(SEQ ID NO: 155)
QQYAYSPLT,
(SEQ ID NO: 168)
QQYSYYPIT,
(SEQ ID NO: 169)
QQYSYTPVT,
(SEQ ID NO: 170)
QQYSYEPVT,
(SEQ ID NO: 171)
QQYAYYSPVT,
(SEQ ID NO: 172)
QQYEYYPMT,
(SEQ ID NO: 188)
QQYSFYPFT,
(SEQ ID NO: 85)
QQYSYSPIT,
(SEQ ID NO: 189)
QQYSAYYQPIT,
(SEQ ID NO: 168)
QQYSYYPIT,
(SEQ ID NO: 190)
QQYEYVPHT,
(SEQ ID NO: 191)
QQYSYMPIT,
(SEQ ID NO: 192)
QQYAYYPVT,
(SEQ ID NO: 191)
QQYSYMPIT,
(SEQ ID NO: 193)
QQYDYRPVT,
(SEQ ID NO: 194)
QQYDFTPMT,
(SEQ ID NO: 195)
QQYSSSSPVT,
(SEQ ID NO: 229)
QQSSYTPIT,
(SEQ ID NO: 230)
QQYAYYPIT,
(SEQ ID NO: 231)
QQYEYYPIT,
(SEQ ID NO: 232)
QQYTYYPIT,
(SEQ ID NO: 168)
QQYSYYPIT,
(SEQ ID NO: 233)
QQSSVEPWT;
CDR-VH1:
(SEQ ID NO: 112)
GFNINWAN,
(SEQ ID NO: 113)
GFNIYLHD,
(SEQ ID NO: 114)
GFNIYWSH,
(SEQ ID NO: 124)
GFNIVGGG,
(SEQ ID NO: 125)
GFNIRSYA,
(SEQ ID NO: 126)
GFNVSHTG,
(SEQ ID NO: 137)
GFNLSYSD,
(SEQ ID NO: 138)
GFNISASG,
(SEQ ID NO: 139)
GFNIYRYG,
(SEQ ID NO: 140)
GFNIYGTM,
(SEQ ID NO: 141)
GFNISYSY,
(SEQ ID NO: 156)
GFNVSAYW,
(SEQ ID NO: 157)
GFNISGYG,
(SEQ ID NO: 158)
GFNVSSVG,
(SEQ ID NO: 159)
GFNVSSYG,
(SEQ ID NO: 173)
GFNFSYGY,
(SEQ ID NO: 174)
GFNVWGPG,
(SEQ ID NO: 175)
GFNVSGSQ,
(SEQ ID NO: 176)
GFNIYGQM,
(SEQ ID NO: 177)
GFNVMYST,
(SEQ ID NO: 196)
GFNFGSY,
(SEQ ID NO: 197)
GFNISDSY,
(SEQ ID NO: 198)
GFNIFSDQ,
(SEQ ID NO: 199)
GFNLSYSY,
(SEQ ID NO: 200)
GFNISYGY,
(SEQ ID NO: 201)
GFNISYQH,
(SEQ ID NO: 202)
GFNLSGYY,
(SEQ ID NO: 203)
GFNVSGQY,
(SEQ ID NO: 204)
GFNVSTSG,
(SEQ ID NO: 205)
GFNISYAK,
(SEQ ID NO: 206)
GFNFSSYV,
(SEQ ID NO: 234)
GFNISQGG,
(SEQ ID NO: 235)
GFNISSTG,
(SEQ ID NO: 236)
GFNFFSTV,
(SEQ ID NO: 237)
GFNLHGYL,
(SEQ ID NO: 238)
GFNLSTHV,
(SEQ ID NO: 239)
GFNVSYYS;
CDR-VH2:
(SEQ ID NO: 115)
ISPPYDYT,
(SEQ ID NO: 116)
IIPAIDYT,
(SEQ ID NO: 117)
ISSFEGYT,
(SEQ ID NO: 127)
IYPQGDYT,
(SEQ ID NO: 128)
VGPGKGYT,
(SEQ ID NO: 128)
VGPGKGYT,
(SEQ ID NO: 142)
VMPDSGHT,
(SEQ ID NO: 143)
IHPLKPYT,
(SEQ ID NO: 144)
LYPYGYST,
(SEQ ID NO: 145)
FKPDSYNT,
(SEQ ID NO: 146)
LLPYDGNT,
(SEQ ID NO: 160)
IYGGSGYT,
(SEQ ID NO: 161)
LYGGSDYT,
(SEQ ID NO: 162)
IYGTSDYT,
(SEQ ID NO: 163)
IAPRRDYT,
(SEQ ID NO: 178)
ISGYTGNT,
(SEQ ID NO: 179)
IHPFSGNT,
(SEQ ID NO: 180)
IPGWSGYT,
(SEQ ID NO: 181)
LSPFSGNT,
(SEQ ID NO: 182)
IYSWSDYT,
(SEQ ID NO: 207)
ISGYSGNT,
(SEQ ID NO: 208)
FSPYSSNT,
(SEQ ID NO: 209)
FNIPYDSYYT,
(SEQ ID NO: 210)
ISGFSGNT,
(SEQ ID NO: 211)
FHYGSGNT,
(SEQ ID NO: 212)
FMPYQGST,
(SEQ ID NO: 213)
FSPYSGYT,
(SEQ ID NO: 214)
ISPVSGNT,
(SEQ ID NO: 215)
IYGAYSGT,
(SEQ ID NO: 216)
LTYWGGYT,
(SEQ ID NO: 217)
VYPDSGGT,
(SEQ ID NO: 240)
VYPGGGQT,
(SEQ ID NO: 241)
LLGGSGNT,
(SEQ ID NO: 242)
IYPWSGST,
(SEQ ID NO: 243)
IYPPNGYT,
(SEQ ID NO: 244)
FYPYVGYT,
(SEQ ID NO: 245)
IYPWNDYT;
and
CDR-VH3:
(SEQ ID NO: 118)
SRSYSYYFDY,
(SEQ ID NO: 119)
SRRDGYYFDY,
(SEQ ID NO: 120)
SRSYSYYMDY,
(SEQ ID NO: 129)
SRDSSYLAFDY,
(SEQ ID NO: 130)
SRNFQSTSHAFDY,
(SEQ ID NO: 131)
SRKTYYAFDY,
(SEQ ID NO: 147)
SRATNIPVYAFDY,
(SEQ ID NO: 148)
SRYSSMYYYYFDY,
(SEQ ID NO: 149)
SRSYAYGYFAY,
(SEQ ID NO: 150)
SRGEVYHYYAFDY,
(SEQ ID NO: 151)
SRAAYSSMDV,
(SEQ ID NO: 164)
SRTHSYWSAFDY,
(SEQ ID NO: 165)
SRTVRYAFDY,
(SEQ ID NO: 166)
SRSSRYSMDY,
(SEQ ID NO: 167)
SRKSSYYFDY,
(SEQ ID NO: 183)
SRAASLSSSYYSAFDV,
(SEQ ID NO: 184)
SRGYSYSAMDY,
(SEQ ID NO: 185)
SRGYSYFAMDY,
(SEQ ID NO: 186)
SRNISYEQSSAFDY,
(SEQ ID NO: 187)
SRGYAHNSFDY,
(SEQ ID NO: 218)
SRSNQSAYSYMDY,
(SEQ ID NO: 219)
SRSQFTFYQYFDY,
(SEQ ID NO: 220)
SRMSVRNAFDY,
(SEQ ID NO: 221)
SRSDSYYTAMDY,
(SEQ ID NO: 222)
SRSNYYYLDY,
(SEQ ID NO: 223)
SRANIYSSHSFFDY,
(SEQ ID NO: 224)
SRTHSSIYHSFDY,
(SEQ ID NO: 225)
SRPMKTSYYGAFDY,
(SEQ ID NO: 226)
SRSQSYTYWSAMDY,
(SEQ ID NO: 227)
SRGEYGTYMDY,
(SEQ ID NO: 228)
SRTSSYYAFDY,
(SEQ ID NO: 246)
SRGYDYSAFDY,
(SEQ ID NO: 247)
SRGLQYSAMDY,
(SEQ ID NO: 248)
SRSRSSNYYFDV,
(SEQ ID NO: 249)
SRGVDYAYLDY,
(SEQ ID NO: 250)
SRGYRYQYMDV,
(SEQ ID NO: 251)
SRGSYYSFDY.

For example, a molecule that can bind to a modified peptide derived from a modified CTNNB polypeptide (e.g., TTAPFLSGK (SEQ ID NO:26)) can include one of each of the CDRs set forth below:

CDR-VL1:
(SEQ ID NO: 33)
QDVNTA;
CDR-VL2:
SAS and SAY;
CDR-VL3:
(SEQ ID NO: 38)
QQSYYSPPT,
(SEQ ID NO: 252)
QQIYTSPIT,
(SEQ ID NO: 253)
QQRAYFPIT,
(SEQ ID NO: 254)
QQQYAYTPIT,
(SEQ ID NO: 255)
QQIHYKPLT;
CDR-VH1:
(SEQ ID NO: 256)
GFNINNTY,
(SEQ ID NO: 257)
GFNFITTG,
(SEQ ID NO: 258)
GFNFSDYG,
(SEQ ID NO: 259)
GFNVWSYG,
(SEQ ID NO: 260)
GFNVAWYS;
CDR-VH2:
(SEQ ID NO: 260)
IYPTDGYT,
(SEQ ID NO: 261)
IGPGSDYT,
(SEQ ID NO: 262)
LIPASGYT,
(SEQ ID NO: 263)
VTPDGSYT,
(SEQ ID NO: 264)
VYGGSSYT;
and
CDR-VH3:
(SEQ ID NO: 265)
SRTYYSYYSAMDV,
(SEQ ID NO: 266)
SRYYYASALDY,
(SEQ ID NO: 267)
SRGWSYYMDY,
(SEQ ID NO: 268)
SRSYGWAMDY,
(SEQ ID NO: 269)
SRDFYSSGMDY.

For example, a molecule that can bind to a modified peptide derived from a modified KRAS polypeptide (e.g., AVGVGKSAL (SEQ ID NO:11)) can include one of each of the CDRs set forth below:

CDR-VL1:
(SEQ ID NO: 33)
QDVNTA;
CDR-VL2:
SAS;
CDR-VL3:
(SEQ ID NO: 270)
QQEWRLPIT,
(SEQ ID NO: 271)
QQGTSTPFT,
(SEQ ID NO: 272)
QQSWRYPMT,
(SEQ ID NO: 273)
QQSYSYPVT,
(SEQ ID NO: 274)
QQGWLYSPFT;
CDR-VH1:
(SEQ ID NO: 275),
GFNVYGNQ,
(SEQ ID NO: 402)
GFNLSYYG,
(SEQ ID NO: 276)
GFNISRYG,
(SEQ ID NO: 277)
GFNIYSSW,
(SEQ ID NO: 157)
GFNISGYG;
CDR-VH2:
(SEQ ID NO: 278)
IYPYSGST,
(SEQ ID NO: 279)
IYPDSGYT,
(SEQ ID NO: 280)
FYPSSSYT,
(SEQ ID NO: 281)
FQPYSGYT,
(SEQ ID NO: 282)
VYGGSGYT;
and
CDR-VH3:
(SEQ ID NO: 283)
SRSAYVAYSYFDY,
(SEQ ID NO: 284)
SRAYLYYYLAY,
(SEQ ID NO: 285)
SRKYYEAMDY,
(SEQ ID NO: 286)
SREYTYYFDY,
(SEQ ID NO: 287)
SRAHSSYYVDY.

For example, a molecule that can bind to a modified peptide derived from a modified H/K/N RAS polypeptide (e.g., ILDTAGHEEY (SEQ ID NO:28), ILDTAGKEEY (SEQ ID NO:30), ILDTAGLEEY (SEQ ID NO:31), ILDTAGREEY (SEQ ID NO:32)) can include one of each of the CDRs set forth below:

CDR-VL1:
(SEQ ID NO: 33)
QDVNTA;
CDR-VL2:
SAS;
CDR-VL3:
(SEQ ID NO: 292)
QQHYYSPVT,
(SEQ ID NO: 296)
QQYAYAPFT,
(SEQ ID NO: 300)
QQAHMIPIT,
(SEQ ID NO: 301)
QQSVYDPIT,
(SEQ ID NO: 302)
QQSYTSPLT,
(SEQ ID NO: 303)
QQGQYSPFT,
(SEQ ID NO: 320)
QQYWYLPTT;
CDR-VH1:
(SEQ ID NO: 289)
GFNIGYYG,
(SEQ ID NO: 293)
GFNIFYQD,
(SEQ ID NO: 297)
GFNVSYSM,
(SEQ ID NO: 305)
GFNFSFPG,
(SEQ ID NO: 306)
GFNISGSW,
(SEQ ID NO: 307),
GFNIYYGV,
(SEQ ID NO: 308)
GFNVSYEY,
(SEQ ID NO: 321)
GFNISWYD;
CDR-VH2:
(SEQ ID NO: 290)
VYPGGGYT,
(SEQ ID NO: 294)
IYPDYDYT,
(SEQ ID NO: 298)
VWGDGGVT,
(SEQ ID NO: 310)
FVGYDGYT,
(SEQ ID NO: 311)
LYPDSDYT,
(SEQ ID NO: 312)
IYPDSSWT,
(SEQ ID NO: 313)
IYGGSDNT,
(SEQ ID NO: 322)
IEPSVGYT;
and
CDR-VH3:
(SEQ ID NO: 291)
SRYYYYGFDY,
(SEQ ID NO: 295)
SRTYSVYMDY,
(SEQ ID NO: 299)
SRGSYYAFDY,
(SEQ ID NO: 316)
SRDYYSFSMDY,
(SEQ ID NO: 317)
SRAHTYAFDY,
(SEQ ID NO: 318)
SRDQDFHYMNYYLSYALDY,
(SEQ ID NO: 319)
SRPLGSYFDY,
(SEQ ID NO: 323)
SRSYPYYYFDY.

Examples of CDRs (e.g., particular combinations of a CDR-VL1, a CDR-VL2, a CDR-VL3, a CDR-VH1, a CDR-VH2, and a CDR-VH3) that can bind to particular modified peptides are shown in Table 2. In some cases, a molecule including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can include any appropriate set of CDR sequences (e.g., any of the CDR sequence sets described herein).

A molecule including one or more antigen-binding domains (e.g., scFvs) that can bind to a modified peptide described herein (e.g., a modified peptide including an amino acid sequence set forth in any one of SEQ ID NO:1, SEQ ID NO: 11, SEQ ID NO: 13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can include any appropriate sequence. For example, a molecule that can bind to a modified peptide derived from a modified EGFR polypeptide (e.g., IMQLMPFGC (SEQ ID NO:13)) can include, without limitation, the scFv sequence set forth in any one of SEQ ID NO:324, SEQ ID NO:325, SEQ ID NO:326, SEQ ID NO:327, SEQ ID NO:328, SEQ ID NO:329, and SEQ ID NO:330. For example, a molecule that can bind to a modified peptide derived from a modified IDH2 polypeptide (e.g., SPNGTIQNIL (SEQ ID NO:1)) can include, without limitation, the scFv sequence set forth in any one of SEQ ID NO:3, SEQ ID NO:4, SEQ ID NO:5, SEQ ID NO:6, and SEQ ID NO:8. For example, a molecule that can bind to a modified peptide derived from a modified p53 polypeptide (e.g., GMNQRPILTI (SEQ ID NO:15) and GMNWRPILTI 1(SEQ ID NO:16)) can include, without limitation, the scFv sequence set forth in any one of SEQ ID NO:331, SEQ ID NO:332, SEQ ID NO:333, SEQ ID NO:334, SEQ ID NO:335, SEQ ID NO:336, and SEQ ID NO:337. For example, a molecule that can bind to a modified peptide derived from a modified KRAS polypeptide (e.g., LVVVGAVGV (SEQ ID NO:18), VVVGACGVGK (SEQ ID NO:20), VVVGADGVGK (SEQ ID NO:21), VVVGAVGVGK (SEQ ID NO:22), and VVGADGVGK (SEQ ID NO:24)) can include, without limitation, the scFv sequence set forth in any one of SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, and SEQ ID NO:374. For example, a molecule that can bind to a modified peptide derived from a modified CTNNB polypeptide (e.g., TTAPFLSGK (SEQ ID NO:26)) can include, without limitation, the scFv sequence set forth in any one of SEQ ID NO:375, SEQ ID NO:376, SEQ ID NO:377, SEQ ID NO:378, SEQ ID NO:379. For example, a molecule that can bind to a modified peptide derived from a modified KRAS polypeptide (e.g., AVGVGKSAL (SEQ ID NO:11)) can include, without limitation, the scFv sequence set forth in any one of SEQ ID NO:380, SEQ ID NO:390, SEQ ID NO:391, SEQ ID NO:392, and SEQ ID NO:393. For example, a molecule that can bind to a modified peptide derived from a modified H/K/N RAS polypeptide (e.g., ILDTAGHEEY (SEQ ID NO:28), ILDTAGKEEY (SEQ ID NO:30), ILDTAGLEEY (SEQ ID NO:31), ILDTAGREEY (SEQ ID NO:32)) can include, without limitation, the scFv sequence set forth in any one of SEQ ID NO:394, SEQ ID NO:395, SEQ ID NO:396, SEQ ID NO:397, SEQ ID NO:398, SEQ ID NO:399, and SEQ ID NO:400. Examples of sequences (e.g., scFv sequences) that can bind to particular modified peptides are shown in Table 3. In some cases, a molecule including one or more antigen-binding domains (e.g., scFvs) that can bind to a modified peptide described herein (e.g., a modified peptide including an amino acid sequence set forth in any one of SEQ ID NO: 1, SEQ ID NO: 11, SEQ ID NO: 13, SEQ ID NO: 15, SEQ ID NO:16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can have a sequence that deviates from a sequence shown in Table 3, sometimes referred to as a variant sequence. For example, a molecule including one or more antigen-binding domains that can bind to a modified peptide described herein can have at least 75% sequence identity (e.g., at least 80% sequence identity, at least 85% sequence identity, at least 90% sequence identity, at least 95% sequence identity, at least 96% sequence identity, at least 97% sequence identity, at least 98% sequence identity, at least 99% sequence identity, or more) to any of the sequences shown in Table 3, provided the variant sequence maintains the ability to bind to a modified peptide described herein. In some cases, a molecule including one or more antigen-binding domains that can bind to a modified peptide described herein can include any appropriate set of CDR sequences described herein, and any sequence deviations from a sequence shown in Table 3 can be in the scaffold sequence(s).

A molecule including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO: 16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be attached (e.g., covalently or non-covalently attached) to a label (e.g., a detectable label). A detectable label can be any appropriate label. In some cases, a label can be used to assist in detecting the presence or absence of one or more modified peptides described herein. For example, a molecule described herein that is labelled can be used in vitro to detect cancer cells (e.g., cancer cells expressing a modified peptide described herein) in a sample obtained from a mammal. In some cases, a label (e.g., a detectable label) can be used to assist in determining the location of one or more modified peptides described herein. For example, molecule described herein that is labelled can be used in vivo to monitor anti-tumor therapy and/or to detect cancer cells (e.g., cancer cells expressing a modified peptide described herein) in a mammal. Examples of labels that can be attached to a molecule described herein include, without limitation, radionuclides, chromophores, enzymes, and fluorescent molecules (e.g., green fluorescent protein).

A molecule including one or more antigen-binding domains (e.g., scFvs) that can bind to a modified peptide described herein (e.g., a modified peptide including an amino acid sequence set forth in any one of SEQ ID NO:1, SEQ ID NO: 11, SEQ ID NO: 13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be attached (e.g., covalently or non-covalently attached) to a therapeutic agent. A therapeutic agent can be any therapeutic agent. In some cases, a therapeutic agent can be an anti-cancer agent. Examples of therapeutic agents that can be attached to a molecule described herein include, without limitation, anti-cancer agents such as monomethyl auristatin E (MMAE), monomethyl auristatin F (MMAF), maytansine, mertansine/emtansine (DM1), ravtansine/soravtansine (DM4), SN-38, calicheamicin, D6.5, dimeric pyrrolobenzodiazepines (PBDs), ricin, pseudomonas exotoxin A, diphtheria toxin, and gelonin.

This document also provides methods for using one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO: 13, SEQ ID NO: 15, SEQ ID NO: 16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32). For example, one or more molecules including one or more antigen-binding domains (e.g., scFvs) that can target (e.g., bind to) one or more modified peptides can be used to assess a mammal having cancer or suspected of having cancer and/or to treat a mammal having a cancer (e.g., a cancer expressing one or more modified peptides). In some cases, one or more molecules includes one or more antigen-binding domains that can bind to a modified peptide can be used to detect the presence or absence of one or more modified peptides in a sample obtained from a mammal having cancer or suspected of having cancer. In some cases, one or more molecules including one or more antigen-binding domains that can bind to a modified peptide can be administered to a mammal having a cancer (e.g., a cancer expressing the modified peptide) to treat the mammal. Administration of one or more molecules including one or more antigen-binding domains that can bind to a modified peptide described herein to a mammal (e.g., human) having a cancer can be effective to treat the mammal.

Any type of mammal can be assessed and/or treated as described herein. Examples of mammals that can be assessed and/or treated as described herein include, without limitation, primates (e.g., humans and non-human primates such as chimpanzees, baboons, or monkeys), dogs, cats, pigs, sheep, rabbits, mice, and rats. In some cases, a mammal can be a human.

A mammal can be assessed and/or treated for any appropriate cancer. In some cases, a cancer can express one or more modified peptides (e.g., one or more MANAs) described herein. A cancer can be a primary cancer. A cancer can be a metastatic cancer. A cancer can include one or more solid tumors. A cancer can include one or more non-solid tumors. Examples of cancers that can be assessed as described herein (e.g., based at least in part on the presence of one or more modified peptides described herein) and/or treated as described herein (e.g, by administering one or more molecules including one or more antigen-binding domains (e.g., scFvs) that can bind to a modified peptide described herein) include, without limitation, blood cancers (e.g., Hodgkin's lymphoma, non-Hodgkin's lymphoma, leukemia such as acute myeloid leukemia (AML), and myeloma), lung cancers, pancreatic cancers, gastric cancers, colon cancers (e.g., colorectal cancers), ovarian cancers, endometrial cancers, biliary tract cancers, liver cancers, bone and soft tissue cancers, breast cancers, prostate cancers, esophageal cancers, stomach cancers, kidney cancers, head and neck cancers, and brain cancers (e.g., glioblastoma multiforme and astrocytomas).

When assessing a mammal having cancer or suspected of having cancer, one or more molecules including one or more antigen-binding domains (e.g., scFvs) that can bind to a modified peptide described herein (e.g., a modified peptide including an amino acid sequence set forth in any one of SEQ ID NO:1, SEQ ID NO: 11, SEQ ID NO: 13, SEQ ID NO: 15, SEQ ID NO:16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be used to assess for the presence or absence of one or more modified peptides described herein. For example, the presence, absence, or level of one or more modified peptides described herein in a sample obtained from a human can be used to determine whether or not the human has a cancer. In some cases, the presence of one or more modified peptides described herein in a sample obtained from a mammal can be used to identify the mammal as having a cancer. For example, a mammal can be identified as having a cancer when a sample obtained from the mammal has one or more modified peptides described herein.

Any appropriate sample obtained from a mammal can be assessed for the presence, absence, or level of one or more modified peptides described herein. For example, biological samples such as tissue samples (e.g., breast tissue), and fluid samples (e.g., blood, serum, plasma, or urine) can be obtained from a mammal and assessed for the presence, absence, or level of one or more modified peptides described herein. Any appropriate method can be used to detect the presence, absence, or level of one or more modified peptides described herein. For example, sequencing techniques including, but not limited to, Sanger sequencing, chemical sequencing, nanopore sequencing, sequencing by ligation (SOLID sequencing), and sequencing with mass spectrometry can be used to determine the presence, absence, or level of one or more modified peptides described herein in a sample obtained from a mammal.

When treating a mammal having cancer, one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO: 15, SEQ ID NO: 16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be administered to a mammal having cancer to treat the mammal. In some cases, a mammal can have a cancer expressing one or more modified peptides described herein. For example, one or more molecules including one or more antigen-binding domains that can bind to a modified peptide described herein can be administered to a mammal having a cancer expressing that modified peptide to treat the mammal. For example, one or more molecules including one or more scFvs that can bind to a modified peptide described herein (e.g., one or more CARs and/or one or more scDbs) can be administered to a mammal having a cancer expressing that modified peptide to treat the mammal.

In some cases, one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be administered to a mammal (e.g., a mammal having a cancer) once or multiple times over a period of time ranging from days to weeks. In some cases, one or more molecules including one or more antigen-binding domains (e.g., scFvs) that can bind to a modified peptide described herein can be formulated into a pharmaceutically acceptable composition for administration to a mammal. For example, an effective amount of one or more molecules including one or more antigen-binding domains (e.g., scFvs) that can bind to a modified peptide described herein can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A composition containing one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO: 13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be designed for oral, parenteral (including subcutaneous, intramuscular, intravenous, and intradermal), or intratumoral administration. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

A composition containing one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be administered using any appropriate technique and to any appropriate location. A composition including one or more molecules including one or more antigen-binding domains (e.g., scFvs) that can bind to a modified peptide described herein can be administered locally (e.g., intratumorally) or systemically. For example, a composition provided herein can be administered locally by intratumoral administration (e.g., injection into tumors) or by administration into biological spaces infiltrated by tumors (e.g. intraspinal administration, intracelebellar administration, intraperitoneal administration and/or pleural administration). For example, a composition provided herein can be administered systemically by oral administration or by intravenous administration (e.g., injection or infusion) to a mammal (e.g., a human).

Effective doses can vary depending on the risk and/or the severity of the cancer, the route of administration, the age and general health condition of the subject, excipient usage, the possibility of co-usage with other therapeutic treatments such as use of other agents, and the judgment of the treating physician. An effective amount of a composition containing one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO: 13, SEQ ID NO:15, SEQ ID NO:16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be any amount that treats a cancer present within the subject without producing significant toxicity to the subject. If a particular subject fails to respond to a particular amount, then the amount of one or more molecules including one or more antigen-binding domains (e.g., scFvs) that can bind to a modified peptide described herein can be increased (e.g., by two-fold, three-fold, four-fold, or more). After receiving this higher amount, the mammal can be monitored for both responsiveness to the treatment and toxicity symptoms, and adjustments made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO: 13, SEQ ID NO: 15, SEQ ID NO: 16, SEQ ID NO: 18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be any frequency that effectively treats a mammal having a cancer without producing significant toxicity to the mammal. For example, the frequency of administration of one or more molecules including one or more antigen-binding domains (e.g., scFvs) that can bind to a modified peptide described herein can be from about two to about three times a week to about two to about three times a year. In some cases, a subject having cancer can receive a single administration of one or more antibodies described herein. The frequency of administration of one or more molecules including one or more antigen-binding domains (e.g., scFvs) that can bind to a modified peptide described herein can remain constant or can be variable during the duration of treatment. A course of treatment with a composition containing one or more molecules including one or more antigen-binding domains (e.g., scFvs) that can bind to a modified peptide described herein can include rest periods. For example, a composition containing one or more molecules including one or more antigen-binding domains (e.g., scFvs) that can bind to a modified peptide described herein can be administered every other month over a two-year period followed by a six-month rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO: 13, SEQ ID NO: 15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be any duration that effectively treats a cancer present within the mammal without producing significant toxicity to the mammal. In some cases, the effective duration can vary from several months to several years. In general, the effective duration for treating a mammal having a cancer can range in duration from about one or two months to five or more years. Multiple factors can influence the actual effective duration used for a particular treatment. For example, an effective duration can vary with the frequency of administration, effective amount, use of multiple treatment agents, route of administration, and severity of the condition being treated.

In certain instances, a cancer within a mammal can be monitored to evaluate the effectiveness of the cancer treatment. Any appropriate method can be used to determine whether or not a mammal having cancer is treated. For example, imaging techniques or laboratory assays can be used to assess the number of cancer cells and/or the size of a tumor present within a mammal. For example, imaging techniques or laboratory assays can be used to assess the location of cancer cells and/or a tumor present within a mammal.

In some cases, one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32) can be administered to a mammal having a cancer as a combination therapy with one or more additional cancer treatments (e.g., anti-cancer agents). A cancer treatment can include any appropriate cancer treatments. In some cases, a cancer treatment can include surgery. In some cases, a cancer treatment can include radiation therapy. In some cases, a cancer treatment can include administration of one or more therapeutic agents (e.g., one or more anti-cancer agents). Examples of anti-cancer agents include, without limitation, platinum compounds (e.g., a cisplatin or carboplatin), taxanes (e.g., paclitaxel, docetaxel, or an albumin bound paclitaxel such as nab-paclitaxel), altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, luteinizing-hormone-releasing hormone (LHRH) agonists (e.g., goserelin and leuprolide), anti-estrogens (e.g., tamoxifen), aromatase inhibitors (e.g., letrozole, anastrozole, and exemestane), angiogenesis inhibitors (e.g., bevacizumab), poly(ADP)-ribose polymerase (PARP) inhibitors (e.g., olaparib, rucaparib, and niraparib), radioactive phosphorus, anti-CTLA-4 antibodies, anti-PD-1 antibodies, anti-PD-L1 antibodies, IL-2 and other cytokines, and any combinations thereof. In cases where one or more molecules including one or more antigen-binding domains (e.g., scFvs) that can bind to a modified peptide described herein are used in combination with one or more additional cancer treatments, the one or more additional cancer treatments can be administered at the same time or independently. For example, a composition including one or more molecules including one or more antigen-binding domains (e.g., scFvs) that can bind to a modified peptide described herein can be administered first, and the one or more additional cancer treatments administered second, or vice versa.

Also provided herein are kits that include one or more molecules including one or more antigen-binding domains (e.g., scFvs) that can bind to a modified peptide described herein (e.g., a modified peptide including an amino acid sequence set forth in any one of SEQ ID NO:1, SEQ ID NO:11, SEQ ID NO:13, SEQ ID NO:15, SEQ ID NO:16, SEQ ID NO:18, SEQ ID NO:20, SEQ ID NO:21, SEQ ID NO:22, SEQ ID NO:24, SEQ ID NO:26, SEQ ID NO:28, SEQ ID NO:30, SEQ ID NO:31, and SEQ ID NO:32). For example, a kit can include a composition (e.g., a pharmaceutically acceptable composition) containing one or more molecules including one or more antigen-binding domains (e.g., scFvs) that can bind to a modified peptide described herein. In some cases, a kit can include instructions for performing any of the methods described herein. In some cases, a kit can include at least one dose of any of the compositions (e.g., pharmaceutical compositions) described herein. In some cases, a kit can provide a means (e.g., a syringe) for administering any of the compositions (e.g., pharmaceutical compositions) described herein.

The invention is further described in the following examples, which do not limit the scope of the invention described in the claims.

### EXAMPLES

### Example 1: Identification of Additional MANAbody Clones and Conversion of MANAbody Clones into T Cell-Based Therapeutic Formats

In this study, two phage display libraries were designed and built, both of which displayed a single chain variable fragment (scFv) on the phage surface. The scFvs present in both libraries were based on the humanized 4D5 (trastuzumab) framework with amino acid variability introduced at key positions of the scFv's complementarity determining regions (CDRs).

Phage display libraries were used to identify scFvs that specifically recognized mutation-containing peptides folded into a complex with a recombinant HLA allele alpha chain and beta-2 microglobulin (b2M). These complexes, also referred to herein as monomers, mimic the natural peptide/HLA complexes on a cancer cell surface.

Peptide-HLA targets can include mutant peptides (e.g., Mutation-Associated Neo-Antigens (MANAs)) shown in Table 1. Complementarity-determining regions (CDRs) that can specifically bind to peptide-HLA targets in Table 1 are shown in Table 2. scFvs that can specifically bind to peptide-HLA targets in Table 1 are shown in Table 3. These scFvs can also be referred to as MANAbodies for their ability to bind to Mutation-Associated Neo-Antigens.

TABLE 2
MANAbody complementarity-determining region (CDR) sequences
of light (L) chains and heavy (H) chains.

| | | | | | | | |
|---|---|---|---|---|---|---|---|
| | Target | | | | | | |
| Target | HLA | | CDR | | | | |
| Peptide(s) | Allele | CDR L1 | L2 | CDR L3 | CDR H1 | CDR H2 | CDR H3 |

1) EGFR T790M(789-797)-A2

| | | | | | | | |
|---|---|---|---|---|---|---|---|
| IMQLMPFGC | HLA-A2 | QDVNTA | SAS | QQYDYAPIT | GFNISWYQ | VTPYSGYT | SRSYTDGFDY |
| (SEQ ID NO: 13) | | (SEQ ID | | (SEQ ID NO: 34) | (SEQ ID NO: 41) | (SEQ ID NO: 48) | (SEQ ID NO: 55) |
| | | NO: 33) | | | | | |
| IMQLMPFGC | HLA-A2 | QDVNTA | SAS | QQSPYYYLPIT | GFNVSWSY | IYGDSGYT | SRGQWEASYYAMDY |
| (SEQ ID NO: 13) | | (SEQ ID | | (SEQ ID NO: 35) | (SEQ ID NO: 42) | (SEQ ID NO: 49) | (SEQ ID NO: 56) |
| | | NO: 33) | | | | | |
| IMQLMPFGC | HLA-A2 | QDVNTA | SAS | QQYYYSPVT | GFNISWNQ | VSPYSGYT | SRSDYYAMDY |
| (SEQ ID NO: 13) | | (SEQ ID | | (SEQ ID NO: 36) | (SEQ ID NO: 43) | (SEQ ID NO: 50) | (SEQ ID NO: 57) |
| | | NO: 33) | | | | | |
| IMQLMPFGC | HLA-A2 | QDVNTA | SAS | QQHYGNPFT | GFNVGYYG | VSGMEGYT | SRDIYGYAMDV |
| (SEQ ID NO: 13) | | (SEQ ID | | (SEQ ID NO: 37) | (SEQ ID NO: 44) | (SEQ ID NO: 51) | (SEQ ID NO: 58) |
| | | NO: 33) | | | | | |
| IMQLMPFGC | HLA-A2 | QDVNTA | SAS | QQSYYSPPT | GFNITSSY | ISPADGYN | SRTDSTAYTAMDV |
| (SEQ ID NO: 13) | | (SEQ ID | | (SEQ ID NO: 38) | (SEQ ID NO: 45) | (SEQ ID NO: 52) | (SEQ ID NO: 59) |
| | | NO: 33) | | | | | |
| IMQLMPFGC | HLA-A2 | QDVNTA | SAS | QQYYSYPPT | GFNINSSY | ISPTDGYY | SRTSDTSYAAMDV |
| (SEQ ID NO: 13) | | (SEQ ID | | (SEQ ID NO: 39) | (SEQ ID NO: 46) | (SEQ ID NO: 53) | (SEQ ID NO: 60) |
| | | NO: 33) | | | | | |
| IMQLMPFGC | HLA-A2 | QDVNTA | SAS | QQYYYYPPT | GFNISTSY | IDPNDGYS | SRTNNTAADAMDV |
| (SEQ ID NO: 13) | | (SEQ ID | | (SEQ ID NO: 40) | (SEQ ID NO: 47) | (SEQ ID NO: 54) | (SEQ ID NO: 61) |
| | | NO: 33) | | | | | |

2) IDH2 R140Q(134-143)-B7

| | | | | | | | |
|---|---|---|---|---|---|---|---|
| SPNGTIQNIL | HLA-B7 | QDVNTA | SAS | QQYSYSPPT | GFNISDTY | ISPRTGYN | SRAYYSYAYAMDV |
| (SEQ ID NO: 1) | | (SEQ ID | | (SEQ ID NO: 62) | (SEQ ID NO: 67) | (SEQ ID NO: 72) | (SEQ ID NO: 77) |
| | | NO: 33) | | | | | |
| SPNGTIQNIL | HLA-B7 | QDVNTA | SAS | QQGKAYWPAT | GFNVGHYR | VSPNGYYT | SRGYSSYAFDY |
| (SEQ ID NO: 1) | | (SEQ ID | | (SEQ ID NO: 63) | (SEQ ID NO: 68) | (SEQ ID NO: 73) | (SEQ ID NO: 78) |
| | | NO: 33) | | | | | |
| SPNGTIQNIL | HLA-B7 | QDVNTA | SAS | QQVYSSPFT | GFNVKYYM | ISPGYDYT | SRSYWRYSVDV |
| (SEQ ID NO: 1) | | (SEQ ID | | (SEQ ID NO: 64) | (SEQ ID NO: 69) | (SEQ ID NO: 74) | (SEQ ID NO: 79) |
| | | NO: 33) | | | | | |
| SPNGTIQNIL | HLA-B7 | QDVNTA | SAS | QQYSLYSPMT | GFNSFLS | IFPSSDYT | SRGKHSSDSNYYMDY |
| (SEQ ID NO: 1) | | (SEQ ID | | (SEQ ID NO: 65) | (SEQ ID NO: 70) | (SEQ ID NO: 75) | (SEQ ID NO: 80) |
| | | NO: 33) | | | | | |
| SPNGTIQNIL | HLA-B7 | QDVNTA | SAS | QQSYYMPFT | GFNIFRGY | ISPHSDYT | SRSYGWAAFDY |
| (SEQ ID NO: 1) | | (SEQ ID | | (SEQ ID NO: 66) | (SEQ ID NO: 71) | (SEQ ID NO: 76) | (SEQ ID NO: 81) |
| | | NO: 33) | | | | | |

3) p53 R248Q/W(245-254)-A2

| | | | | | | | |
|---|---|---|---|---|---|---|---|
| GMNQRPILTI | HLA-A2 | QDVNTA | SAS | QQSGYAPIT | GFNISYYS | VDPDSDYT | SRSWIHMFSMDY |
| (SEQ ID NO: 15) | | (SEQ ID | | (SEQ ID NO: 82) | (SEQ ID NO: 89) | (SEQ ID NO: 96) | (SEQ ID NO: 103) |
| | | NO: 33) | | | | | |
| GMNWRPILTI | HLA-A2 | QDVNTA | SAS | QQYSYAPIT | GFNIGYYT | VSPWSYST | SRDHWDEAFDV |
| (SEQ ID NO: 16) | | (SEQ ID | | (SEQ ID NO: 83) | (SEQ ID NO: 90) | (SEQ ID NO: 97) | (SEQ ID NO: 104) |
| | | NO: 33) | | | | | |
| GMNQRPILTI | HLA-A2 | QDVNTA | SAS | QQSLYGPFT | GFNIAYEY | IGPDSGYT | SRVWYYSTYGMDY |
| (SEQ ID NO: 15) | | (SEQ ID | | (SEQ ID NO: 84) | (SEQ ID NO: 91) | (SEQ ID NO: 98) | (SEQ ID NO: 105) |
| | | NO: 33) | | | | | |
| GMNWRPILTI | HLA-A2 | QDVNTA | SAS | QQYSYSPIT | GFNLFGYG | IGPYYYYT | SRENYDMAMDY |
| (SEQ ID NO: 16) | | (SEQ ID | | (SEQ ID NO: 85) | (SEQ ID NO: 92) | (SEQ ID NO: 99) | (SEQ ID NO: 106) |
| | | NO: 33) | | | | | |
| GMNWRPILTI | HLA-A2 | QDVNTA | SAS | QQSGYQPDT | GFNISWYA | IWPDSDWT | SRYYYSSAFDV |
| (SEQ ID NO: 16) | | (SEQ ID | | (SEQ ID NO: 86) | (SEQ ID NO: 93) | (SEQ ID NO: 100) | (SEQ ID NO: 107) |
| | | NO: 33) | | | | | |
| GMNQRPILTI | HLA-A2 | QDVNTA | SAS | QQYLYQPWT | GFNIDYYG | LYGGSDST | SRQYSAYFDY |
| (SEQ ID NO: 15), | | (SEQ ID | | (SEQ ID NO: 87) | (SEQ ID NO: 94) | (SEQ ID NO: 101) | (SEQ ID NO: 108) |
| GMNWRPILTI | | NO: 33) | | | | | |
| (SEQ ID NO: 16) | | | | | | | |
| GMNQRPILTI | HLA-A2 | QDVNTA | SAS | QQGLYYPWT | GFNV SYS S | IWPD SGQT | SRSSYFDAMDY |
| (SEQ ID NO: 15), | | (SEQ ID | | (SEQ ID NO: 88) | (SEQ ID NO: 95) | (SEQ ID NO: 102) | (SEQ ID NO: 109) |
| GMNWRPILTI | | NO: 33) | | | | | |
| (SEQ ID NO: 16) | | | | | | | |

4) KRAS G12V(6-14)-A2

| | | | | | | | |
|---|---|---|---|---|---|---|---|
| LVVVGAVGV | HLA-A2 | QDVNTA | SAS | QQWYSSPVT | GFNINWAN | ISPPYDYT | SRSYSYYFDY |
| (SEQ ID NO: 18) | | (SEQ ID | | (SEQ ID NO: 110) | (SEQ ID NO: 112) | (SEQ ID NO: 115) | (SEQ ID NO: 118) |
| | | NO: 33) | | | | | |
| LVVVGAVGV | HLA-A2 | QDVNTA | SAS | QQYYSRPVT | GFNIYLHD | IIPAIDYT | SRRDGYYFDY |
| (SEQ ID NO: 18) | | (SEQ ID | | (SEQ ID NO: 111) | (SEQ ID NO: 113) | (SEQ ID NO: 116) | (SEQ ID NO: 119) |
| | | NO: 33) | | | | | |
| LVVVGAVGV | HLA-A2 | QDVNTA | SAS | QQWYSSPVT | GFNIYWSH | ISSFEGYT | SRSYSYYMDY |
| (SEQ ID NO: 18) | | (SEQ ID | | (SEQ ID NO: 110) | (SEQ ID NO: 114) | (SEQ ID NO: 117) | (SEQ ID NO: 120) |
| | | NO: 33) | | | | | |

5) KRAS G12C/D/V(7-16)-A3

| | | | | | | | |
|---|---|---|---|---|---|---|---|
| VVVGACGVGK | HLA-A3 | QDVNTA | SAS | QQSYGSGSPWT | GFNIVGGG | IYPQGDYT | SRDSSYLAFDY |
| (SEQ ID NO: 20), | | (SEQ ID | | (SEQ ID NO: 121) | (SEQ ID NO: 124) | (SEQ ID NO: 127) | (SEQ ID NO: 129) |
| VVVGAVGVGK | | NO: 33) | | | | | |
| (SEQ ID NO: 22) | | | | | | | |
| VVVGACGVGK | HLA-A3 | QDVNTA | SAS | QQTYYSPWT | GFNIRSYA | VGPGKGYT | SRNFQSTSHAFDY |
| (SEQ ID NO: 20), | | (SEQ ID | | (SEQ ID NO: 122) | (SEQ ID NO: 125) | (SEQ ID NO: 128) | (SEQ ID NO: 130) |
| VVVGADGVGK | | NO: 33) | | | | | |
| (SEQ ID NO: 21), | | | | | | | |
| VVVGAVGVGK | | | | | | | |
| (SEQ ID NO: 22) | | | | | | | |
| VVVGACGVGK | HLA-A3 | QDVNTA | SAS | QQYYYPPIT | GFNVSHTG | VGPGKGYT | SRKTYYAFDY |
| (SEQ ID NO: 20), | | (SEQ ID | | (SEQ ID NO: 123) | (SEQ ID NO: 126) | (SEQ ID NO: 128) | (SEQ ID NO: 131) |
| VVVGADGVGK | | NO: 33) | | | | | |
| (SEQ ID NO: 21), | | | | | | | |
| VVVGAVGVGK | | | | | | | |
| (SEQ ID NO: 22) | | | | | | | |

6) KRAS G12V(7-16)-A3

| | | | | | | | |
|---|---|---|---|---|---|---|---|
| VVVGAVGVGK | HLA-A3 | QDVNTA | SAS | QQSYYYFRPIT | GFNLSYSD | VMPDSGHT | SRATNIPVYAFDY |
| (SEQ ID NO: 22) | | (SEQ ID | | (SEQ ID NO: 132) | (SEQ ID NO: 137) | (SEQ ID NO: 142) | (SEQ ID NO: 147) |
| | | NO: 33) | | | | | |
| VVVGAVGVGK | HLA-A3 | QDVNTA | SAS | QQASYYYPLT | GFNISASG | IHPLKPYT | SRYSSMYYYYFDY |
| (SEQ ID NO: 22) | | (SEQ ID | | (SEQ ID NO: 133) | (SEQ ID NO: 138) | (SEQ ID NO: 143) | (SEQ ID NO: 148) |
| | | NO: 33) | | | | | |
| VVVGAVGVGK | HLA-A3 | QDVNTA | SAS | QQKSEYSPWT | GFNIYRYG | LYPYGYST | SRSYAYGYFAY |
| (SEQ ID NO: 22) | | (SEQ ID | | (SEQ ID NO: 134) | (SEQ ID NO: 139) | (SEQ ID NO: 144) | (SEQ ID NO: 149) |
| | | NO: 33) | | | | | |
| VVVGAVGVGK | HLA-A3 | QDVNTA | SAS | QQSGYIPFT | GFNIYGTM | FKPDSYNT | SRGEVYHYYAFDY |
| (SEQ ID NO: 22) | | (SEQ ID | | (SEQ ID NO: 135) | (SEQ ID NO: 140) | (SEQ ID NO: 145) | (SEQ ID NO: 150) |
| | | NO: 33) | | | | | |
| VVVGAVGVGK | HLA-A3 | QDVNTA | SAS | QQGAYYRPFT | GFNISYSY | LLPYDGNT | SRAAYSSMDV |
| (SEQ ID NO: 22) | | (SEQ ID | | (SEQ ID NO: 136) | (SEQ ID NO: 141) | (SEQ ID NO: 146) | (SEQ ID NO: 151) |
| | | NO: 33) | | | | | |

7) KRAS G12D(7-16)-A3

| | | | | | | | |
|---|---|---|---|---|---|---|---|
| VVVGADGVGK | HLA-A3 | QDVNTA | SAS | QQYMYSPVT | GFNVSAYW | IYGGSGYT | SRTHSYWSAFDY |
| (SEQ ID NO: 21) | | (SEQ ID | | (SEQ ID NO: 152) | (SEQ ID NO: 156) | (SEQ ID NO: 160) | (SEQ ID NO: 164) |
| | | NO: 33) | | | | | |
| VVVGADGVGK | HLA-A3 | QDVNTA | SAS | QQSSSSPIT | GFNISGYG | LYGGSDYT | SRTVRYAFDY |
| (SEQ ID NO: 21) | | (SEQ ID | | (SEQ ID NO: 153) | (SEQ ID NO: 157) | (SEQ ID NO: 161) | (SEQ ID NO: 165) |
| | | NO: 33) | | | | | |
| VVVGADGVGK | HLA-A3 | QDVNTA | SAS | QQSSASPLT | GFNVSSVG | IYGTSDYT | SRSSRYSMDY |
| (SEQ ID NO: 21) | | (SEQ ID | | (SEQ ID NO: 154) | (SEQ ID NO: 158) | (SEQ ID NO: 162) | (SEQ ID NO: 166) |
| | | NO: 33) | | | | | |
| VVVGADGVGK | HLA-A3 | QDVNTA | SAS | QQYAYSPLT | GFNVSSYG | IAPRRDYT | SRKSSYYFDY |
| (SEQ ID NO: 21) | | (SEQ ID | | (SEQ ID NO: 155) | (SEQ ID NO: 159) | (SEQ ID NO: 163) | (SEQ ID NO: 167) |
| | | NO: 33) | | | | | |

8) KRAS G12D(7-16)-A11

| | | | | | | | |
|---|---|---|---|---|---|---|---|
| VVVGADGVGK | HLA-A11 | QDVNTA | SAS | QQYSYYPIT | GFNFSYGY | ISGYTGNT | SRAASLSSSYYSAFDV |
| (SEQ ID NO: 21) | | (SEQ ID | | (SEQ ID NO: 168) | (SEQ ID NO: 173) | (SEQ ID NO: 178) | (SEQ ID NO: 183) |
| | | NO: 33) | | | | | |
| VVVGADGVGK | HLA-A11 | QDVNTA | SAS | QQYSYTPVT | GFNVWGPG | IHPFSGNT | SRGYSYSAMDY |
| (SEQ ID NO: 21) | | (SEQ ID | | (SEQ ID NO: 169) | (SEQ ID NO: 174) | (SEQ ID NO: 179) | (SEQ ID NO: 184) |
| | | NO: 33) | | | | | |
| VVVGADGVGK | HLA-A11 | QDVNTA | SAS | QQYSYEPVT | GFNVSGSQ | IPGWSGYT | SRGYSYFAMDY |
| (SEQ ID NO: 21) | | (SEQ ID | | (SEQ ID NO: 170) | (SEQ ID NO: 175) | (SEQ ID NO: 180) | (SEQ ID NO: 185) |
| | | NO: 33) | | | | | |
| VVVGADGVGK | HLA-A11 | QDVNTA | SAS | QQYAYYSPVT | GFNIYGQM | LSPFSGNT | SRNISYEQSSAFDY |
| (SEQ ID NO: 21) | | (SEQ ID | | (SEQ ID NO: 171) | (SEQ ID NO: 176) | (SEQ ID NO: 181) | (SEQ ID NO: 186) |
| | | NO: 33) | | | | | |
| VVVGADGVGK | HLA-A11 | QDVNTA | SAS | QQYEYYPMT | GFNVMYST | IYSWSDYT | SRGYAHNSFDY |
| (SEQ ID NO: 21) | | (SEQ ID | | (SEQ ID NO: 172) | (SEQ ID NO: 177) | (SEQ ID NO: 182) | (SEQ ID NO: 187) |
| | | NO: 33) | | | | | |

9) KRAS G12D(8-16)-A11

| | | | | | | | |
|---|---|---|---|---|---|---|---|
| VVGADGVGK | HLA-A11 | QDVNTA | SAS | QQYSFYPFT | GFNFGSY | ISGYSGNT | SRSNQSAYSYMDY |
| (SEQ ID NO: 24) | | (SEQ ID | | (SEQ ID NO: 188) | (SEQ ID NO: 196) | (SEQ ID NO: 207) | (SEQ ID NO: 218) |
| | | NO: 33) | | | | | |
| VVGADGVGK | HLA-A11 | QDVNTA | SAS | QQYSYSPIT | GFNISDSY | FSPYSSNT | SRSQFTFYQYFDY |
| (SEQ ID NO: 24) | | (SEQ ID | | (SEQ ID NO: 85) | (SEQ ID NO: 197) | (SEQ ID NO: 208) | (SEQ ID NO: 219) |
| | | NO: 33) | | | | | |
| VVGADGVGK | HLA-A11 | QDVNTA | SAY | QQYSAYYQPIT | GFNIFSDQ | FMPYDSYYT | SRMSVRNAFDY |
| (SEQ ID NO: 24) | | (SEQ ID | | (SEQ ID NO: 189) | (SEQ ID NO: 198) | (SEQ ID NO: 209) | (SEQ ID NO: 220) |
| | | NO: 33) | | | | | |
| VVGADGVGK | HLA-A11 | QDVNTA | SAS | QQYSYYPIT | GFNLSYSY | ISGFSGNT | SRSDSYYTAMDY |
| (SEQ ID NO: 24) | | (SEQ ID | | (SEQ ID NO: 168) | (SEQ ID NO: 199) | (SEQ ID NO: 210) | (SEQ ID NO: 221) |
| | | NO: 33) | | | | | |
| VVGADGVGK | HLA-A11 | QDVNTA | SAS | QQYEYVPHT | GFNISYGY | FHYGSGNT | SRSNYYYLDY |
| (SEQ ID NO: 24) | | (SEQ ID | | (SEQ ID NO: 190) | (SEQ ID NO: 200) | (SEQ ID NO: 211) | (SEQ ID NO: 222) |
| | | NO: 33) | | | | | |
| VVGADGVGK | HLA-A11 | QDVNTA | SAS | QQYSYMPIT | GFNISYQH | FMPYQGST | SRANIYSSHSFFDY |
| (SEQ ID NO: 24) | | (SEQ ID | | (SEQ ID NO: 191) | (SEQ ID NO: 201) | (SEQ ID NO: 212) | (SEQ ID NO: 223) |
| | | NO: 33) | | | | | |
| VVGADGVGK | HLA-A11 | QDVNTA | SAS | QQYAYYPVT | GFNLSGYY | FSPYSGYT | SRTHSSIYHSFDY |
| (SEQ ID NO: 24) | | (SEQ ID | | (SEQ ID NO: 192) | (SEQ ID NO: 202) | (SEQ ID NO: 213) | (SEQ ID NO: 224) |
| | | NO: 33) | | | | | |
| VVGADGVGK | HLA-A11 | QDVNTA | SAS | QQYSYMPIT | GFNVSGQY | ISPVSGNT | SRPMKTSYYGAFDY |
| (SEQ ID NO: 24) | | (SEQ ID | | (SEQ ID NO: 191) | (SEQ ID NO: 203) | (SEQ ID NO: 214) | (SEQ ID NO: 225) |
| | | NO: 33) | | | | | |
| VVGADGVGK | HLA-A11 | QDVNTA | SAS | QQYDYRPVT | GFNVSTSG | IYGAYSGT | SRSQSYTYWSAMDY |
| (SEQ ID NO: 24) | | (SEQ ID | | (SEQ ID NO: 193) | (SEQ ID NO: 204) | (SEQ ID NO: 215) | (SEQ ID NO: 226) |
| | | NO: 33) | | | | | |
| VVGADGVGK | HLA-A11 | QDVNTA | SAS | QQYDFTPMT | GFNISYAK | LTYWGGYT | SRGEYGTYMDY |
| (SEQ ID NO: 24) | | (SEQ ID | | (SEQ ID NO: 194) | (SEQ ID NO: 205) | (SEQ ID NO: 216) | (SEQ ID NO: 227) |
| | | NO: 33) | | | | | |
| VVGADGVGK | HLA-A11 | QDVNTA | SAS | QQYSSSSPVT | GFNFSSYV | VYPDSGGT | SRTSSYYAFDY |
| (SEQ ID NO: 24) | | (SEQ ID | | (SEQ ID NO: 195) | (SEQ ID NO: 206) | (SEQ ID NO: 217) | (SEQ ID NO: 228) |
| | | NO: 33) | | | | | |

10) KRAS G12V(7-16)-A11

| | | | | | | | |
|---|---|---|---|---|---|---|---|
| VVVGAVGVGK | HLA-A11 | QDVNTA | SAS | QQSSYTPIT | GFNISQGG | VYPGGGQT | SRGYDYSAFDY |
| (SEQ ID NO: 22) | | (SEQ ID | | (SEQ ID NO: 229) | (SEQ ID NO: 234) | (SEQ ID NO: 240) | (SEQ ID NO: 246) |
| | | NO: 33) | | | | | |
| VVVGAVGVGK | HLA-A11 | QDVNTA | SAS | QQYAYYPIT | GFNISSTG | LLGGSGNT | SRGLQYSAMDY |
| (SEQ ID NO: 22) | | (SEQ ID | | (SEQ ID NO: 230) | (SEQ ID NO: 235) | (SEQ ID NO: 241) | (SEQ ID NO: 247) |
| | | NO: 33) | | | | | |
| VVVGAVGVGK | HLA-A11 | QDVNTA | SAS | QQYEYYPIT | GFNFFSTV | IYPWSGST | SRSRSSNYYFDV |
| (SEQ ID NO: 22) | | | | (SEQ ID NO: 231) | (SEQ ID NO: 236) | (SEQ ID NO: 242) | (SEQ ID NO: 248) |
| VVVGAVGVGK | HLA-A11 | QDVNTA | SAS | QQYTYYPIT | GFNLHGYL | IYPPNGYT | SRGVDYAYLDY |
| (SEQ ID NO: 22) | | (SEQ ID | | (SEQ ID NO: 232) | (SEQ ID NO: 237) | (SEQ ID NO: 243) | (SEQ ID NO: 249) |
| | | NO: 33) | | | | | |
| VVVGAVGVGK | HLA-A11 | QDVNTA | SAS | QQYSYYPIT | GFNLSTHV | FYPYVGYT | SRGYRYQYMDV |
| (SEQ ID NO: 22) | | (SEQ ID | | (SEQ ID NO: 168) | (SEQ ID NO: 238) | (SEQ ID NO: 244) | (SEQ ID NO: 250) |
| | | NO: 33) | | | | | |
| VVVGAVGVGK | HLA-A11 | QDVNTA | SAS | QQSSVEPWT | GFNVSYYS | IYPWNDYT | SRGSYYSFDY |
| (SEQ ID NO: 22) | | (SEQ ID | | (SEQ ID NO: 233) | (SEQ ID NO: 239) | (SEQ ID NO: 245) | (SEQ ID NO: 251) |
| | | NO: 33) | | | | | |

11) CTNNB S45F(41-49)-A3

| | | | | | | | |
|---|---|---|---|---|---|---|---|
| TTAPFLSGK | HLA-A3 | QDVNTA | SAS | QQSYYSPPT | GFNINNTY | IYPTDGYT | SRTYYSYYSAMDV |
| (SEQ ID NO: 26) | | (SEQ ID | | (SEQ ID NO: 38) | (SEQ ID NO: 256) | (SEQ ID NO: 260) | (SEQ ID NO: 265) |
| | | NO: 33) | | | | | |
| TTAPFLSGK | HLA-A3 | QDVNTA | SAY | QQIYTSPIT | GFNFITTG | IGPGSDYT | SRYYYASALDY |
| (SEQ ID NO: 26) | | (SEQ ID | | (SEQ ID NO: 252) | (SEQ ID NO: 257) | (SEQ ID NO: 261) | (SEQ ID NO: 266) |
| | | NO: 33) | | | | | |
| TTAPFLSGK | HLA-A3 | QDVNTA | SAS | QQRAYFPIT | GFNFSDYG | LIPASGYT | SRGWSYYMDY |
| (SEQ ID NO: 26) | | (SEQ ID | | (SEQ ID NO: 253) | (SEQ ID NO: 258) | (SEQ ID NO: 262) | (SEQ ID NO: 267) |
| | | NO: 33) | | | | | |
| TTAPFLSGK | HLA-A3 | QDVNTA | SAS | QQQYAYTPIT | GFNVWSYG | VTPDGSYT | SRSYGWAMDY |
| (SEQ ID NO: 26) | | (SEQ ID | | (SEQ ID NO: 254) | (SEQ ID NO: 259) | (SEQ ID NO: 263) | (SEQ ID NO: 268) |
| | | NO: 33) | | | | | |
| TTAPFLSGK | HLA-A3 | QDVNTA | SAY | QQIHYKPLT | GFNVAWYS | VYGGSSYT | SRDFYSSGMDY |
| (SEQ ID NO: 26) | | (SEQ ID | | (SEQ ID NO: 255) | (SEQ ID NO: 260) | (SEQ ID NO: 264) | (SEQ ID NO: 269) |
| | | NO: 33) | | | | | |

12) KRAS G12V(11-19)-B7

| | | | | | | | |
|---|---|---|---|---|---|---|---|
| AVGVGKSAL | HLA-B7 | QDVNTA | SAS | QQEWRLPIT | GFNVYGNQ | IYPYSGST | SRSAYVAYSYFDY |
| (SEQ ID NO: 11) | | (SEQ ID | | (SEQ ID NO: 270) | (SEQ ID NO: 275) | (SEQ ID NO: 278) | (SEQ ID NO: 283) |
| | | NO: 33) | | | | | |
| AVGVGKSAL | HLA-B7 | QDVNTA | SAS | QQGTSTPFT | GFNLSYYG | IYPDSGYT | SRAYLYYYLAY |
| (SEQ ID NO: 11) | | (SEQ ID | | (SEQ ID NO: 271) | (SEQ ID NO: 402) | (SEQ ID NO: 279) | (SEQ ID NO: 284) |
| | | NO: 33) | | | | | |
| AVGVGKSAL | HLA-B7 | QDVNTA | SAS | QQSWRYPMT | GFNISRYG | FYPSSSYT | SRKYYEAMDY |
| (SEQ ID NO: 11) | | (SEQ ID | | (SEQ ID NO: 272) | (SEQ ID NO: 276) | (SEQ ID NO: 280) | (SEQ ID NO: 285) |
| | | NO: 33) | | | | | |
| AVGVGKSAL | HLA-B7 | QDVNTA | SAS | QQSYSYPVT | GFNIYSSW | FQPYSGYT | SREYTYYFDY |
| (SEQ ID NO: 11) | | (SEQ ID | | (SEQ ID NO: 273) | (SEQ ID NO: 277) | (SEQ ID NO: 281) | (SEQ ID NO: 286) |
| | | NO: 33) | | | | | |
| AVGVGKSAL | HLA-B7 | QDVNTA | SAS | QQGWLYSPFT | GFNISGYG | VYGGSGYT | SRAHSSYYVDY |
| (SEQ ID NO: 11) | | (SEQ ID | | (SEQ ID NO: 274) | (SEQ ID NO: 157) | (SEQ ID NO: 282) | (SEQ ID NO: 287) |
| | | NO: 33) | | | | | |

13) H/K/N RAS Q61H(55-64)-A1

| | | | | | | | |
|---|---|---|---|---|---|---|---|
| ILDTAGHEEY | HLA-A1 | QDVNTA | SAS | QQGYFYYPNT | GFNIGYYG | VYPGGGYT | SRYYYYGFDY |
| (SEQ ID NO: 28) | | (SEQ ID | | (SEQ ID NO: 288) | (SEQ ID NO: 289) | (SEQ ID NO: 290) | (SEQ ID NO: 291) |
| | | NO: 33) | | | | | |

14) H/K/N RAS Q61K(55-64)-A1

| | | | | | | | |
|---|---|---|---|---|---|---|---|
| ILDTAGKEEY | HLA-A1 | QDVNTA | SAS | QQHYYSPVT | GFNIFYQD | IYPDYDYT | SRTYSVYMDY |
| (SEQ ID NO: 30) | | (SEQ ID | | (SEQ ID NO: 292) | (SEQ ID NO: 293) | (SEQ ID NO: 294) | (SEQ ID NO: 295) |
| | | NO: 33) | | | | | |

15) H/K/N Q61L(55-64)-A1

| | | | | | | | |
|---|---|---|---|---|---|---|---|
| ILDTAGLEEY | HLA-A1 | QDVNTA | SAS | QQYAYAPFT | GFNVSYSM | VWGDGGVT | SRGSYYAFDY |
| (SEQ ID NO: 31) | | (SEQ ID | | (SEQ ID NO: 296) | (SEQ ID NO: 297) | (SEQ ID NO: 298) | (SEQ ID NO: 299) |
| | | NO: 33) | | | | | |

16) H/K/N RAS Q61R(55-64)-A1

| | | | | | | | |
|---|---|---|---|---|---|---|---|
| ILDTAGREEY | HLA-A1 | QDVNTA | SAS | QQAHMIPIT | GFNFSFPG | FVGYDGYT | SRDYYSFSMDY |
| (SEQ ID NO: 32) | | (SEQ ID | | (SEQ ID NO: 300) | (SEQ ID NO: 305) | (SEQ ID NO: 310) | (SEQ ID NO: 316) |
| | | NO: 33) | | | | | |
| ILDTAGREEY | HLA-A1 | QDVNTA | SAS | QQSVYDPIT | GFNISGSW | LYPDSDYT | SRAHTYAFDY |
| (SEQ ID NO: 32) | | (SEQ ID | | (SEQ ID NO: 301) | (SEQ ID NO: 306) | (SEQ ID NO: 311) | (SEQ ID NO: 317) |
| | | NO: 33) | | | | | |
| ILDTAGREEY | HLA-A1 | QDVNTA | SAS | QQSYTSPLT | GFNIYYGV | IYPDSSWT | SRDQDFHYMNYYLSYA |
| (SEQ ID NO: 32) | | (SEQ ID | | (SEQ ID NO: 302) | (SEQ ID NO: 307) | (SEQ ID NO: 312) | LDY |
| | | NO: 33) | | | | | (SEQ ID NO: 318) |
| ILDTAGREEY | HLA-A1 | QDVNTA | SAS | QQGQYSPFT | GFNVSYEY | IYGGSDNT | SRPLGSYFDY |
| (SEQ ID NO: 32) | | (SEQ ID | | (SEQ ID NO: 303) | (SEQ ID NO: 308) | (SEQ ID NO: 313) | (SEQ ID NO: 319) |
| | | NO: 33) | | | | | |
| ILDTAGREEY | HLA-A1 | QDVNTA | SAS | QQYWYLPTT | GFNISWYD | IEPSVGYT | SRSYPYYYFDY |
| (SEQ ID NO: 32) | | (SEQ ID | | (SEQ ID NO: 320) | (SEQ ID NO: 321) | (SEQ ID NO: 322) | (SEQ ID NO: 323) |
| | | NO: 33) | | | | | |

TABLE 3
MANAbody scFv sequences.

| | | | | |
|---|---|---|---|---|
| | Target | | | |
| Target | HLA | | | SEQ |
| Peptide(s) | Allele | ScFc clone name | scFv sequence | ID NO: |

1) EGFR T790M(789-797)-A2

| | | | | |
|---|---|---|---|---|
| IMQLMPFGC | HLA-A2 | EGFR_T790M_A2_cl1 | DIQMTQSPSSLSASVGDRVTITCRASQDVNTAVAWYQQ | 324 |
| (SEQ ID NO: 13) | | | KPGKAPKLLIYSASFLYSGVPSRFSGSRSGTDFTLTIS | |
| | | | SLQPEDFATYYCQQYDYAPITFGQGTKVEIKRTGGGSG | |
| | | | GGGSGGGASEVQLVESGGGLVQPGGSLRLSCAASGFNI | |
| | | | SWYQMHWVRQAPGKGLEWVALVTPYSGYTYYADSVKGR | |
| | | | FTISADTSKNTAYLQMNSLRAEDTAVYYCSRSYTDGFD | |
| | | | YWGQGTLVTVSS | |
| IMQLMPFGC | HLA-A2 | EGFR_T790M_A2_cl5 | DIQMTQSPSSLSASVGDRVTITCRASQDVNTAVAWYQQ | 325 |
| (SEQ ID NO: 13) | | | KPGKAPKLLIYSASFLYSGVPSRFSGSRSGTDFTLTIS | |
| | | | SLQPEDFATYYCQQSPYYYLPITFGQGTKVEIKRTGGG | |
| | | | SGGGGSGGGASEVQLVESGGGLVQPGGSLRLSCAASGF | |
| | | | NVSWSYMHWVRQAPGKGLEWVANIYGDSGYTHYADSVK | |
| | | | GRFTISADTSKNTAYLQMNSLRAEDTAVYYCSRGQWEA | |
| | | | SYYAMDYWGQGTLVTVSS | |
| IMQLMPFGC | HLA-A2 | EGFR_T790M_A2_cl18 | DIQMTQSPSSLSASVGDRVTITCRASQDVNTAVAWYQQ | 326 |
| (SEQ ID NO: 13) | | | KPGKAPKLLIYSASFLYSGVPSRFSGSRSGTDFTLTIS | |
| | | | SLQPEDFATYYCQQYYYSPVTFGQGTKVEIKRTGGGSG | |
| | | | GGGSGGGASEVQLVESGGGLVQPGGSLRLSCAASGFNI | |
| | | | SWNQMHWVRQAPGKGLEWVALVSPYSGYTYYADSVKGR | |
| | | | FTISADTSKNTAYLQMNSLRAEDTAVYYCSRSDYYAMD | |
| | | | YWGQGTLVTVSS | |
| IMQLMPFGC | HLA-A2 | EGFR_T790M_A2_cl23 | DIQMTQSPSSLSASVGDRVTITCRASQDVNTAVAWYQQ | 327 |
| (SEQ ID NO: 13) | | | KPGKAPKLLIYSASFLYSGVPSRFSGSRSGTDFTLTIS | |
| | | | SLQPEDFATYYCQQHYGNPFTFGQGTKVEIKRTGGGSG | |
| | | | GGGSGGGASEVQLVESGGGLVQPGGSLRLSCAASGFNV | |
| | | | GYYGMHWVRQAPGKGLEWVAFVSGMEGYTSYADSVKGR | |
| | | | FTISADTSKNTAYLQMNSLRAEDTAVYYCSRDIYGYAM | |
| | | | DVWGQGTLVTVSS | |
| IMQLMPFGC | HLA-A2 | EGFR_T790M_A2_D2D6 | DIQMTQSPSSLSASVGDRVTITCRASQDVNTAVAWYQQ | 328 |
| (SEQ ID NO: 13) | | | KPGKAPKLLIYSASFLYSGVPSRFSGSRSGTDFTLTIS | |
| | | | SLQPEDFATYYCQQSYYSPPTFGQGTKVEIKRTGGGSG | |
| | | | GGGSGGGASEVQLVESGGGLVQPGGSLRLSCAASGFNI | |
| | | | TSSYIHWVRQAPGKGLEWVAYISPADGYNRYADSVKGR | |
| | | | FTISADTSKNTAYLQMNSLRAEDTAVYYCSRTDSTAYT | |
| | | | AMDVWGQGTLVTVSS | |
| IMQLMPFGC | HLA-A2 | EGFR_T790M_A2_D2D8 | DIQMTQSPSSLSASVGDRVTITCRASQDVNTAVAWYQQ | 329 |
| (SEQ ID NO: 13) | | | KPGKAPKLLIYSASFLYSGVPSRFSGSRSGTDFTLTIS | |
| | | | SLQPEDFATYYCQQYYSYPPTFGQGTKVEIKRTGGGSG | |
| | | | GGGSGGGASEVQLVESGGGLVQPGGSLRLSCAASGFNI | |
| | | | NSSYIHWVRQAPGKGLEWVAYISPTDGYYRYADSVKGR | |
| | | | FTISADTSKNTAYLQMNSLRAEDTAVYYCSRTSDTSYA | |
| | | | AMDVWGQGTLVTVSS | |
| IMQLMPFGC | HLA-A2 | EGFR_T790M_A2_D3E6 | DIQMTQSPSSLSASVGDRVTITCRASQDVNTAVAWYQQ | 330 |
| (SEQ ID NO: 13) | | | KPGKAPKLLIYSASFLYSGVPSRFSGSRSGTDFTLTIS | |
| | | | SLQPEDFATYYCQQYYYYPPTFGQGTKVEIKRTGGGSG | |
| | | | GGGSGGGASEVQLVESGGGLVQPGGSLRLSCAASGFNI | |
| | | | STSYIHWVRQAPGKGLEWVATIDPNDGYSRYADSVKGR | |
| | | | FTISADTSKNTAYLQMNSLRAEDTAVYYCSRTNNTAAD | |
| | | | AMDVWGQGTLVTVSS | |

2) IDH2 R140Q(134-143)-B7

| | | | | |
|---|---|---|---|---|
| SPNGTIQNIL | HLA-B7 | IDH2_R140Q_B7_D4 | DIQMTQSPSSLSASVGDRVTITCRASQDVNTAVAWYQQ | 3 |
| (SEQ ID NO: 1) | | | KPGKAPKLLIYSASFLYSGVPSRFSGSRSGTDFTLTIS | |
| | | | SLQPEDFATYYCQQYSYSPPTFGQGTKVEIKRTGGGSG | |
| | | | GGGSGGGASEVQLVESGGGLVQPGGSLRLSCAASGFNI | |
| | | | SDTYIHWVRQAPGKGLEWVASISPRTGYNRYADSVKGR | |
| | | | FTISADTSKNTAYLQMNSLRAEDTAVYYCSRAYYSYAY | |
| | | | AMDVWGQGTLVTVSS | |
| SPNGTIQNIL | HLA-B7 | IDH2_R140Q_B7_cl29 | DIQMTQSPSSLSASVGDRVTITCRASQDVNTAVAWYQQ | 4 |
| (SEQ ID NO: 1) | | | KPGKAPKLLIYSASFLYSGVPSRFSGSRSGTDFTLTIS | |
| | | | SLQPEDFATYYCQQGKAYWPATFGQGTKVEIKRTGGGS | |
| | | | GGGGSGGGASEVQLVESGGGLVQPGGSLRLSCAASGFN | |
| | | | VGHYRMHWVRQAPGKGLEWVAMVSPNGYYTYYADSVKG | |
| | | | RFTISADTSKNTAYLQMNSLRAEDTAVYYCSRGYSSYA | |
| | | | FDYWGQGTLVTVSS | |
| SPNGTIQNIL | HLA-B7 | IDH2_R140Q_B7_cl1 | DIQMTQSPSSLSASVGDRVTITCRASQDVNTAVAWYQQ | 5 |
| (SEQ ID NO: 1) | | | KPGKAPKLLIYSASFLYSGVPSRFSGSRSGTDFTLTIS | |
| | | | SLQPEDFATYYCQQVYSSPFTFGQGTKVEIKRTGGGSG | |
| | | | GGGSGGGASEVQLVESGGGLVQPGGSLRLSCAASGFNV | |
| | | | KYYMMHWVRQAPGKGLEWVAAISPGYDYTYYADSVKGR | |
| | | | FTISADTSKNTAYLQMNSLRAEDTAVYYCSRSYWRYSV | |
| | | | DVWGQGTLVTVSS | |
| SPNGTIQNIL | HLA-B7 | IDH2_R140Q_B7_cl3 | DIQMTQSPSSLSASVGDRVTITCRASQDVNTAVAWYQQ | 6 |
| (SEQ ID NO: 1) | | | KPGKAPKLLIYSASFLYSGVPSRFSGSRSGTDFTLTIS | |
| | | | SLQPEDFATYYCQQYSLYSPMTFGQGTKVEIKRTGGGS | |
| | | | GGGGSGGGASEVQLVESGGGLVQPGGSLRLSCAASGFN | |
| | | | SFLSIHWVRQAPGKGLEWVAHIFPSSDYTSYADSVKGR | |
| | | | FTISADTSKNTAYLQMNSLRAEDTAVYYCSRGKHSSDS | |
| | | | NYYMDYWGQGTLVTVSS | |
| SPNGTIQNIL | HLA-B7 | IDH2_R140Q_B7_cl8 | DIQMTQSPSSLSASVGDRVTITCRASQDVNTAVAWYQQ | 8 |
| (SEQ ID NO: 1) | | | KPGKAPKLLIYSASFLYSGVPSRFSGSRSGTDFTLTIS | |
| | | | SLQPEDFATYYCQQSYYMPFTFGQGTKVEIKRTGGGSG | |
| | | | GGGSGGGASEVQLVESGGGLVQPGGSLRLSCAASGFNI | |
| | | | FRGYMHWVRQAPGKGLEWVAMISPHSDYTSYADSVKGR | |
| | | | FTISADTSKNTAYLQMNSLRAEDTAVYYCSRSYGWAAF | |
| | | | DYWGQGTLVTVSS | |

3) p53 R248Q/W(245-254)-A2

| | | | | |
|---|---|---|---|---|
| GMNQRPILTI | HLA-A2 | p53_R248Q_A2_cl0 | DIQMTQSPSSLSASVGDRVTITCRASQDVNTAVAWYQQ | 331 |
| (SEQ ID NO: 15) | | | KPGKAPKLLIYSASFLYSGVPSRFSGSRSGTDFTLTIS | |
| | | | SLQPEDFATYYCQQSGYAPITFGQGTKVEIKRTGGGSG | |
| | | | GGGSGGGASEVQLVESGGGLVQPGGSLRLSCAASGFNI | |
| | | | SYYSMHWVRQAPGKGLEWVADVDPDSDYTEYADSVKGR | |
| | | | FTISADTSKNTAYLQMNSLRAEDTAVYYCSRSWIHMFS | |
| | | | MDYWGQGTLVTVSS | |
| GMNWRPILTI | HLA-A2 | p53_R248W_A2_cl2 | DIQMTQSPSSLSASVGDRVTITCRASQDVNTAVAWYQQ | 332 |
| (SEQ ID NO: 16) | | | KPGKAPKLLIYSASFLYSGVPSRFSGSRSGTDFTLTIS | |
| | | | SLQPEDFATYYCQQYSYAPITFGQGTKVEIKRTGGGSG | |
| | | | GGGSGGGASEVQLVESGGGLVQPGGSLRLSCAASGFNI | |
| | | | GYYTMHWVRQAPGKGLEWVAEVSPWSYSTSYADSVKGR | |
| | | | FTISADTSKNTAYLQMNSLRAEDTAVYYCSRDHWDEAF | |
| | | | DVWGQGTLVTVSS | |
| GMNQRPILTI | HLA-A2 | p53_R248Q_A2_cl4 | DIQMTQSPSSLSASVGDRVTITCRASQDVNTAVAWYQQ | 333 |
| (SEQ ID NO: 15) | | | KPGKAPKLLIYSASFLYSGVPSRFSGSRSGTDFTLTIS | |
| | | | SLQPEDFATYYCQQSLYGPFTFGQGTKVEIKRTGGGSG | |
| | | | GGGSGGGASEVQLVESGGGLVQPGGSLRLSCAASGFNI | |
| | | | AYEYMHWVRQAPGKGLEWVALIGPDSGYTYYADSVKGR | |
| | | | FTISADTSKNTAYLQMNSLRAEDTAVYYCSRVWYYSTY | |
| | | | GMDYWGQGTLVTVSS | |
| GMNWRPILTI | HLA-A2 | p53_R248W_A2_cl8 | DIQMTQSPSSLSASVGDRVTITCRASQDVNTAVAWYQQ | 334 |
| (SEQ ID NO: 16) | | | KPGKAPKLLIYSASFLYSGVPSRFSGSRSGTDFTLTIS | |
| | | | SLQPEDFATYYCQQYSYSPITFGQGTKVEIKRTGGGSG | |
| | | | GGGSGGGASEVQLVESGGGLVQPGGSLRLSCAASGFNL | |
| | | | FGYGMHWVRQAPGKGLEWVAEIGPYYYYTSYADSVKGR | |
| | | | FTISADTSKNTAYLQMNSLRAEDTAVYYCSRENYDMAM | |
| | | | DYWGQGTLVTVSS | |
| GMNWRPILTI | HLA-A2 | p53_R248W_A2_cl11 | DIQMTQSPSSLSASVGDRVTITCRASQDVNTAVAWYQQ | 335 |
| (SEQ ID NO: 16) | | | KPGKAPKLLIYSASFLYSGVPSRFSGSRSGTDFTLTIS | |
| | | | SLQPEDFATYYCQQSGYQPDTFGQGTKVEIKRTGGGSG | |
| | | | GGGSGGGASEVQLVESGGGLVQPGGSLRLSCAASGFNI | |
| | | | SWYAMHWVRQAPGKGLEWVAEIWPDSDWTYYADSVKGR | |
| | | | FTISADTSKNTAYLQMNSLRAEDTAVYYCSRYYYSSAF | |
| | | | DVWGQGTLVTVSS | |
| GMNQRPILTI | HLA-A2 | p53_R248QW_A2_cl14 | DIQMTQSPSSLSASVGDRVTITCRASQDVNTAVAWYQQ | 336 |
| (SEQ ID NO: 15), | | | KPGKAPKLLIYSASFLYSGVPSRFSGSRSGTDFTLTIS | |
| GMNWRPILTI | | | SLQPEDFATYYCQQYLYQPWTFGQGTKVEIKRTGGGSG | |
| (SEQ ID NO: 16) | | | GGGSGGGASEVQLVESGGGLVQPGGSLRLSCAASGFNI | |
| | | | DYYGMHWVRQAPGKGLEWVASLYGGSDSTDYADSVKGR | |
| | | | FTISADTSKNTAYLQMNSLRAEDTAVYYCSRQYSAYFD | |
| | | | YWGQGTLVTVSS | |
| GMNQRPILTI | HLA-A2 | p53_R248QW_A2_cl17 | DIQMTQSPSSLSASVGDRVTITCRASQDVNTAVAWYQQ | 337 |
| (SEQ ID NO: 15), | | | KPGKAPKLLIYSASFLYSGVPSRFSGSRSGTDFTLTIS | |
| GMNWRPILTI | | | SLQPEDFATYYCQQGLYYPWTFGQGTKVEIKRTGGGSG | |
| (SEQ ID NO: 16) | | | GGGSGGGASEVQLVESGGGLVQPGGSLRLSCAASGFNV | |
| | | | SYSSIHWVRQAPGKGLEWVAEIWPDSGQTWYADSVKGR | |
| | | | FTISADTSKNTAYLQMNSLRAEDTAVYYCSRSSYFDAM | |
| | | | DYWGQGTLVTVSS | |

4) KRAS G12V(6-14)-A2

| | | | | |
|---|---|---|---|---|
| LVVVGAVGV | HLA-A2 | KRAS_G12V_A2_A1 | DIQMTQSPSSLSASVGDRVTITCRASQDVNTAVAWYQQ | 338 |
| (SEQ ID NO: 18) | | | KPGKAPKLLIYSASFLYSGVPSRFSGSRSGTDFTLTIS | |
| | | | SLQPEDFATYYCQQWYSSPVTFGQGTKVEIKRTGGGSG | |
| | | | GGGSGGGASEVQLVESGGGLVQPGGSLRLSCAASGFNI | |
| | | | NWANMHWVRQAPGKGLEWVAQISPPYDYTNYADSVKGR | |
| | | | FTISADTSKNTAYLQMNSLRAEDTAVYYCSRSYSYYFD | |
| | | | YWGQGTLVTVSS | |
| LVVVGAVGV | HLA-A2 | KRAS_G12V_A2_C1 | DIQMTQSPSSLSASVGDRVTITCRASQDVNTAVAWYQQ | 339 |
| (SEQ ID NO: 18) | | | KPGKAPKLLIYSASFLYSGVPSRFSGSRSGTDFTLTIS | |
| | | | SLQPEDFATYYCQQYYSRPVTFGQGTKVEIKRTGGGSG | |
| | | | GGGSGGGASEVQLVESGGGLVQPGGSLRLSCAASGFNI | |
| | | | YLHDMHWVRQAPGKGLEWVAQIIPAIDYTNYADSVKGR | |
| | | | FTISADTSKNTAYLQMNSLRAEDTAVYYCSRRDGYYFD | |
| | | | YWGQGTLVTVSS | |
| LVVVGAVGV | HLA-A2 | KRAS_G12V_A2_A5 | DIQMTQSPSSLSASVGDRVTITCRASQDVNTAVAWYQQ | 340 |
| (SEQ ID NO: 18) | | | KPGKAPKLLIYSASFLYSGVPSRFSGSRSGTDFTLTIS | |
| | | | SLQPEDFATYYCQQWYSSPVTFGQGTKVEIKRTGGGSG | |
| | | | GGGSGGGASEVQLVESGGGLVQPGGSLRLSCAASGFNI | |
| | | | YWSHMHWVRQAPGKGLEWVAIISSFEGYTYYADSVKGR | |
| | | | FTISADTSKNTAYLQMNSLRAEDTAVYYCSRSYSYYMD | |
| | | | YWGQGTLVTVSS | |

5) KRAS G12C/D/V(7-16)-A3

| | | | | |
|---|---|---|---|---|
| VVVGACGVGK | HLA-A3 | KRAS_G12CV_A3_cl5 | DIQMTQSPSSLSASVGDRVTITCRASQDVNTAVAWYQQ | 341 |
| (SEQ ID NO: 20), | | | KPGKAPKLLIYSASFLYSGVPSRFSGSRSGTDFTLTIS | |
| VVVGAVGVGK | | | SLQPEDFATYYCQQSYGSGSPWTFGQGTKVEIKRTGGG | |
| (SEQ ID NO: 22) | | | SGGGGSGGGASEVQLVESGGGLVQPGGSLRLSCAASGF | |
| | | | NIVGGGIHWVRQAPGKGLEWVAKIYPQGDYTYYADSVK | |
| | | | GRFTISADTSKNTAYLQMNSLRAEDTAVYYCSRDSSYL | |
| | | | AFDYWGQGTLVTVSS | |
| VVVGACGVGK | HLA-A3 | KRAS_G12CDV_A3_cl9 | DIQMTQSPSSLSASVGDRVTITCRASQDVNTAVAWYQQ | 342 |
| (SEQ ID NO: 20), | | | KPGKAPKLLIYSASFLYSGVPSRFSGSRSGTDFTLTIS | |
| VVVGADGVGK | | | SLQPEDFATYYCQQTYYSPWTFGQGTKVEIKRTGGGSG | |
| (SEQ ID NO: 21), | | | GGGSGGGASEVQLVESGGGLVQPGGSLRLSCAASGFNI | |
| VVVGAVGVGK | | | RSYAMHWVRQAPGKGLEWVAQVGPGKGYTYYADSVKGR | |
| (SEQ ID NO: 22) | | | FTISADTSKNTAYLQMNSLRAEDTAVYYCSRNFQSTSH | |
| | | | AFDYWGQGTLVTVSS | |
| VVVGACGVGK | HLA-A3 | KRAS_G12CDV_A3_cl18 | DIQMTQSPSSLSASVGDRVTITCRASQDVNTAVAWYQQ | 343 |
| (SEQ ID NO: 20), | | | KPGKAPKLLIYSASFLYSGVPSRFSGSRSGTDFTLTIS | |
| VVVGADGVGK | | | SLQPEDFATYYCQQYYYPPITFGQGTKVEIKRTGGGSG | |
| (SEQ ID NO: 21), | | | GGGSGGGASEVQLVESGGGLVQPGGSLRLSCAASGFNV | |
| VVVGAVGVGK | | | SHTGMHWVRQAPGKGLEWVAVVGPGKGYTYYADSVKGR | |
| (SEQ ID NO: 22) | | | FTISADTSKNTAYLQMNSLRAEDTAVYYCSRKTYYAFD | |
| | | | YWGQGTLVTVSS | |

6) KRAS G12V(7-16)-A3

| | | | | |
|---|---|---|---|---|
| VVVGAVGVGK | HLA-A3 | KRAS_G12VA3_cl2 | DIQMTQSPSSLSASVGDRVTITCRASQDVNTAVAWYQQ | 344 |
| (SEQ ID NO: 22) | | | KPGKAPKLLIYSASFLYSGVPSRFSGSRSGTDFTLTIS | |
| | | | SLQPEDFATYYCQQSYYYFRPITFGQGTKVEIKRTGGG | |
| | | | SGGGGSGGGASEVQLVESGGGLVQPGGSLRLSCAASGF | |
| | | | NLSYSDIHWVRQAPGKGLEWVAVVMPDSGHTNYADSVK | |
| | | | GRFTISADTSKNTAYLQMNSLRAEDTAVYYCSRATNIP | |
| | | | VYAFDYWGQGTLVTVSS | |
| VVVGAVGVGK | HLA-A3 | KRAS G12V_A3_V12 | DIQMTQSPSSLSASVGDRVTITCRASQDVNTAVAWYQQ | 345 |
| (SEQ ID NO: 22) | | | KPGKAPKLLIYSASFLYSGVPSRFSGSRSGTDFTLTIS | |
| | | | SLQPEDFATYYCQQASYYYPLTFGQGTKVEIKRTGGGS | |
| | | | GGGGSGGGASEVQLVESGGGLVQPGGSLRLSCAASGFN | |
| | | | ISASGMHWVRQAPGKGLEWVADIHPLKPYTNYADSVKG | |
| | | | RFTISADTSKNTAYLQMNSLRAEDTAVYYCSRYSSMYY | |
| | | | YYFDYWGQGTLVTVSS | |
| VVVGAVGVGK | HLA-A3 | KRAS G12V_A3_cl20 | DIQMTQSPSSLSASVGDRVTITCRASQDVNTAVAWYQQ | 346 |
| (SEQ ID NO: 22) | | | KPGKAPKLLIYSASFLYSGVPSRFSGSRSGTDFTLTIS | |
| | | | SLQPEDFATYYCQQKSEYSPWTFGQGTKVEIKRTGGGS | |
| | | | GGGGSGGGASEVQLVESGGGLVQPGGSLRLSCAASGFN | |
| | | | IYRYGIHWVRQAPGKGLEWVAVLYPYGYSTSYADSVKG | |
| | | | RFTISADTSKNTAYLQMNSLRAEDTAVYYCSRSYAYGY | |
| | | | FAYWGQGTLVTVSS | |
| VVVGAVGVGK | HLA-A3 | KRAS G12V_A3_cl21 | DIQMTQSPSSLSASVGDRVTITCRASQDVNTAVAWYQQ | 347 |
| (SEQ ID NO: 22) | | | KPGKAPKLLIYSASFLYSGVPSRFSGSRSGTDFTLTIS | |
| | | | SLQPEDFATYYCQQSGYIPFTFGQGTKVEIKRTGGGSG | |
| | | | GGGSGGGASEVQLVESGGGLVQPGGSLRLSCAASGFNI | |
| | | | YGTMMHWVRQAPGKGLEWVAQFKPDSYNTYYADSVKGR | |
| | | | FTISADTSKNTAYLQMNSLRAEDTAVYYCSRGEVYHYY | |
| | | | AFDYWGQGTLVTVSS | |
| VVVGAVGVGK | HLA-A3 | KRAS G12V_A3_cl22 | DIQMTQSPSSLSASVGDRVTITCRASQDVNTAVAWYQQ | 348 |
| (SEQ ID NO: 22) | | | KPGKAPKLLIYSASFLYSGVPSRFSGSRSGTDFTLTIS | |
| | | | SLQPEDFATYYCQQGAYYRPFTFGQGTKVEIKRTGGGS | |
| | | | GGGGSGGGASEVQLVESGGGLVQPGGSLRLSCAASGFN | |
| | | | ISYSYMHWVRQAPGKGLEWVATLLPYDGNTYYADSVKG | |
| | | | RFTISADTSKNTAYLQMNSLRAEDTAVYYCSRAAYSSM | |
| | | | DVWGQGTLVTVSS | |

7) KRAS G12D(7-16)-A3

| | | | | |
|---|---|---|---|---|
| VVVGADGVGK | HLA-A3 | KRAS_G12D_A3_cl11 | DIQMTQSPSSLSASVGDRVTITCRASQDVNTAVAWYQQ | 349 |
| (SEQ ID NO: 21) | | | KPGKAPKLLIYSASFLYSGVPSRFSGSRSGTDFTLTIS | |
| | | | SLQPEDFATYYCQQYMYSPVTFGQGTKVEIKRTGGGSG | |
| | | | GGGSGGGASEVQLVESGGGLVQPGGSLRLSCAASGFNV | |
| | | | SAYWNIHWVRQAPGKGLEWVAQIYGGSGYTMYADSVKG | |
| | | | RFTISADTSKNTAYLQMNSLRAEDTAVYYCSRTHSYWS | |
| | | | AFDYWGQGTLVTVSS | |
| VVVGADGVGK | HLA-A3 | KRAS_G12D_A3_D12 | DIQMTQSPSSLSASVGDRVTITCRASQDVNTAVAWYQQ | 350 |
| (SEQ ID NO: 21) | | | KPGKAPKLLIYSASFLYSGVPSRFSGSRSGTDFTLTIS | |
| | | | SLQPEDFATYYCQQSSSSPITFGQGTKVEIKRTGGGSG | |
| | | | GGGSGGGASEVQLVESGGGLVQPGGSLRLSCAASGFNI | |
| | | | SGYGMHWVRQAPGKGLEWVAYLYGGSDYTNYADSVKGR | |
| | | | FTISADTSKNTAYLQMNSLRAEDTAVYYCSRTVRYAFD | |
| | | | YWGQGTLVTVSS | |
| VVVGADGVGK | HLA-A3 | KRAS_G12D_A3_D15 | DIQMTQSPSSLSASVGDRVTITCRASQDVNTAVAWYQQ | 351 |
| (SEQ ID NO: 21) | | | KPGKAPKLLIYSASFLYSGVPSRFSGSRSGTDFTLTIS | |
| | | | SLQPEDFATYYCQQSSASPLTFGQGTKVEIKRTGGGSG | |
| | | | GGGSGGGASEVQLVESGGGLVQPGGSLRLSCAASGFNV | |
| | | | SSVGNIHWVRQAPGKGLEWVAYIYGTSDYTYYADSVKG | |
| | | | RFTISADTSKNTAYLQMNSLRAEDTAVYYCSRSSRYSM | |
| | | | DYWGQGTLVTVSS | |
| VVVGADGVGK | HLA-A3 | KRAS_G12D_A3_D26 | DIQMTQSPSSLSASVGDRVTITCRASQDVNTAVAWYQQ | 352 |
| (SEQ ID NO: 21) | | | KPGKAPKLLIYSASFLYSGVPSRFSGSRSGTDFTLTIS | |
| | | | SLQPEDFATYYCQQYAYSPLTFGQGTKVEIKRTGGGSG | |
| | | | GGGSGGGASEVQLVESGGGLVQPGGSLRLSCAASGFNV | |
| | | | SSYGMHWVRQAPGKGLEWVAFIAPRRDYTSYADSVKGR | |
| | | | FTISADTSKNTAYLQMNSLRAEDTAVYYCSRKSSYYFD | |
| | | | YWGQGTLVTVSS | |

8) KRAS G12D(7-16)-A11

| | | | | |
|---|---|---|---|---|
| VVVGADGVGK | HLA-A11 | KRAS_G12D_A11_D3 | DIQMTQSPSSLSASVGDRVTITCRASQDVNTAVAWYQQ | 353 |
| (SEQ ID NO: 21) | | | KPGKAPKLLIYSASFLYSGVPSRFSGSRSGTDFTLTIS | |
| | | | SLQPEDFATYYCQQYSYYPITFGQGTKVEIKRTGGGSG | |
| | | | GGGSGGGASEVQLVESGGGLVQPGGSLRLSCAASGFNF | |
| | | | SYGYMHWVRQAPGKGLEWVAWISGYTGNTYYADSVKGR | |
| | | | FTISADTSKNTAYLQMNSLRAEDTAVYYCSRAASLSSS | |
| | | | YYSAFDVWGQGTLVTVSS | |
| VVVGADGVGK | HLA-A11 | KRAS_G12D_A11_D14 | DIQMTQSPSSLSASVGDRVTITCRASQDVNTAVAWYQQ | 354 |
| (SEQ ID NO: 21) | | | KPGKAPKLLIYSASFLYSGVPSRFSGSRSGTDFTLTIS | |
| | | | SLQPEDFATYYCQQYSYTPVTFGQGTKVEIKRTGGGSG | |
| | | | GGGSGGGASEVQLVESGGGLVQPGGSLRLSCAASGFNV | |
| | | | WGPGMHWVRQAPGKGLEWVARIHPFSGNTYYADSVKGR | |
| | | | FTISADTSKNTAYLQMNSLRAEDTAVYYCSRGYSYSAM | |
| | | | DYWGQGTLVTVSS | |
| VVVGADGVGK | HLA-A11 | KRAS_G12D_A11_D18 | DIQMTQSPSSLSASVGDRVTITCRASQDVNTAVAWYQQ | 355 |
| (SEQ ID NO: 21) | | | KPGKAPKLLIYSASFLYSGVPSRFSGSRSGTDFTLTIS | |
| | | | SLQPEDFATYYCQQYSYEPVTFGQGTKVEIKRTGGGSG | |
| | | | GGGSGGGASEVQLVESGGGLVQPGGSLRLSCAASGFNV | |
| | | | SGSQMHWVRQAPGKGLEWVARIPGWSGYTYYADSVKGR | |
| | | | FTISADTSKNTAYLQMNSLRAEDTAVYYCSRGYSYFAM | |
| | | | DYWGQGTLVTVSS | |
| VVVGADGVGK | HLA-A11 | KRAS_G12D_A11_D21 | DIQMTQSPSSLSASVGDRVTITCRASQDVNTAVAWYQQ | 356 |
| (SEQ ID NO: 21) | | | KPGKAPKLLIYSASFLYSGVPSRFSGSRSGTDFTLTIS | |
| | | | SLQPEDFATYYCQQYAYYSPVTFGQGTKVEIKRTGGGS | |
| | | | GGGGSGGGASEVQLVESGGGLVQPGGSLRLSCAASGFN | |
| | | | IYGQMMHWVRQAPGKGLEWVAFLSPFSGNTYYADSVKG | |
| | | | RFTISADTSKNTAYLQMNSLRAEDTAVYYCSRNISYEQ | |
| | | | SSAFDYWGQGTLVTVSS | |
| VVVGADGVGK | HLA-A11 | KRAS_G12D_A11_D22 | DIQMTQSPSSLSASVGDRVTITCRASQDVNTAVAWYQQ | 357 |
| (SEQ ID NO: 21) | | | KPGKAPKLLIYSASFLYSGVPSRFSGSRSGTDFTLTIS | |
| | | | SLQPEDFATYYCQQYEYYPMTFGQGTKVEIKRTGGGSG | |
| | | | GGGSGGGASEVQLVESGGGLVQPGGSLRLSCAASGFNV | |
| | | | MYSTMHWVRQAPGKGLEWVASIYSWSDYTYYADSVKGR | |
| | | | FTISADTSKNTAYLQMNSLRAEDTAVYYCSRGYAHNSF | |
| | | | DYWGQGTLVTVSS | |

9) KRAS G12D(8-16)-A11

| | | | | |
|---|---|---|---|---|
| VVGADGVGK | HLA-A11 | KRAS_G12D_A11_cl4 | DIQMTQSPSSLSASVGDRVTITCRASQDVNTAVAWYQQ | 358 |
| (SEQ ID NO: 24) | | | KPGKAPKLLIYSASFLYSGVPSRFSGSRSGTDFTLTIS | |
| | | | SLQPEDFATYYCQQYSFYPFTFGQGTKVEIKRTGGGSG | |
| | | | GGGSGGGASEVQLVESGGGLVQPGGSLRLSCAASGFNF | |
| | | | GSYIHWVRQAPGKGLEWVAIISGYSGNTYYADSVKGRF | |
| | | | TISADTSKNTAYLQMNSLRAEDTAVYYCSRSNQSAYSY | |
| | | | MDYWGQGTLVTVSS | |
| VVGADGVGK | HLA-A11 | KRAS_G12D_A11_cl6 | DIQMTQSPSSLSASVGDRVTITCRASQDVNTAVAWYQQ | 359 |
| (SEQ ID NO: 24) | | | KPGKAPKLLIYSASFLYSGVPSRFSGSRSGTDFTLTIS | |
| | | | SLQPEDFATYYCQQYSYSPITFGQGTKVEIKRTGGGSG | |
| | | | GGGSGGGASEVQLVESGGGLVQPGGSLRLSCAASGFNI | |
| | | | SDSYMHWVRQAPGKGLEWVATFSPYSSNTWYADSVKGR | |
| | | | FTISADTSKNTAYLQMNSLRAEDTAVYYCSRSQFTFYQ | |
| | | | YFDYWGQGTLVTVSS | |
| VVGADGVGK | HLA-A11 | KRAS_G12D_A11_cl7 | DIQMTQSPSSLSASVGDRVTITCRASQDVNTAVAWYQQ | 360 |
| (SEQ ID NO: 24) | | | KPGKAPKLLIYSAYFLYSGVPSRFSGSRSGTDFTLTIS | |
| | | | SLQPEDFATYYCQQYSAYYQPITFGQGTKVEIKRTGGG | |
| | | | SGGGGSGGGASEVQLVESGGGLVQPGGSLRLSCAASGF | |
| | | | NIFSDQMHWVRQAPGKGLEWVAGFMPYDSYYTNYADSV | |
| | | | KGRFTISADTSKNTAYLQMNSLRAEDTAVYYCSRMSVR | |
| | | | NAFDYWGQGTLVTVSS | |
| VVGADGVGK | HLA-A11 | KRAS_G12D_A11_cl9 | DIQMTQSPSSLSASVGDRVTITCRASQDVNTAVAWYQQ | 361 |
| (SEQ ID NO: 24) | | | KPGKAPKLLIYSASFLYSGVPSRFSGSRSGTDFTLTIS | |
| | | | SLQPEDFATYYCQQYSYYPITFGQGTKVEIKRTGGGSG | |
| | | | GGGSGGGASEVQLVESGGGLVQPGGSLRLSCAASGFNL | |
| | | | SYSYNIHWVRQAPGKGLEWVAVISGFSGNTYYADSVKG | |
| | | | RFTISADTSKNTAYLQMNSLRAEDTAVYYCSRSDSYYT | |
| | | | AMDYWGQGTLVTVSS | |
| VVGADGVGK | HLA-A11 | KRAS_G12D_A11_cl10 | DIQMTQSPSSLSASVGDRVTITCRASQDVNTAVAWYQQ | 362 |
| (SEQ ID NO: 24) | | | KPGKAPKLLIYSASFLYSGVPSRFSGSRSGTDFTLTIS | |
| | | | SLQPEDFATYYCQQYEYVPHTFGQGTKVEIKRTGGGSG | |
| | | | GGGSGGGASEVQLVESGGGLVQPGGSLRLSCAASGFNI | |
| | | | SYGYMHWVRQAPGKGLEWVAKFHYGSGNTYYADSVKGR | |
| | | | FTISADTSKNTAYLQMNSLRAEDTAVYYCSRSNYYYLD | |
| | | | YWGQGTLVTVSS | |
| VVGADGVGK | HLA-A11 | KRAS_G12D_A11_cl12 | DIQMTQSPSSLSASVGDRVTITCRASQDVNTAVAWYQQ | 363 |
| (SEQ ID NO: 24) | | | KPGKAPKLLIYSASFLYSGVPSRFSGSRSGTDFTLTIS | |
| | | | SLQPEDFATYYCQQYSYMPITFGQGTKVEIKRTGGGSG | |
| | | | GGGSGGGASEVQLVESGGGLVQPGGSLRLSCAASGFNI | |
| | | | SYQHIHWVRQAPGKGLEWVAVFMPYQGSTYYADSVKGR | |
| | | | FTISADTSKNTAYLQMNSLRAEDTAVYYCSRANIYSSH | |
| | | | SFFDYWGQGTLVTVSS | |
| VVGADGVGK | HLA-A11 | KRAS_G12D_A11_cl14 | DIQMTQSPSSLSASVGDRVTITCRASQDVNTAVAWYQQ | 364 |
| (SEQ ID NO: 24) | | | KPGKAPKLLIYSASFLYSGVPSRFSGSRSGTDFTLTIS | |
| | | | SLQPEDFATYYCQQYAYYPVTFGQGTKVEIKRTGGGSG | |
| | | | GGGSGGGASEVQLVESGGGLVQPGGSLRLSCAASGFNL | |
| | | | SGYYMHWVRQAPGKGLEWVAWFSPYSGYTYYADSVKGR | |
| | | | FTISADTSKNTAYLQMNSLRAEDTAVYYCSRTHSSIYH | |
| | | | SFDYWGQGTLVTVSS | |
| VVGADGVGK | HLA-A11 | KRAS_G12D_A11_cl15 | DIQMTQSPSSLSASVGDRVTITCRASQDVNTAVAWYQQ | 365 |
| (SEQ ID NO: 24) | | | KPGKAPKLLIYSASFLYSGVPSRFSGSRSGTDFTLTIS | |
| | | | SLQPEDFATYYCQQYSYMPITFGQGTKVEIKRTGGGSG | |
| | | | GGGSGGGASEVQLVESGGGLVQPGGSLRLSCAASGFNV | |
| | | | SGQYMHWVRQAPGKGLEWVAVISPVSGNTYYADSVKGR | |
| | | | FTISADTSKNTAYLQMNSLRAEDTAVYYCSRPMKTSYY | |
| | | | GAFDYWGQGTLVTVSS | |
| VVGADGVGK | HLA-A11 | KRAS_G12D_A11_cl17 | DIQMTQSPSSLSASVGDRVTITCRASQDVNTAVAWYQQ | 366 |
| (SEQ ID NO: 24) | | | KPGKAPKLLIYSASFLYSGVPSRFSGSRSGTDFTLTIS | |
| | | | SLQPEDFATYYCQQYDYRPVTFGQGTKVEIKRTGGGSG | |
| | | | GGGSGGGASEVQLVESGGGLVQPGGSLRLSCAASGFNV | |
| | | | STSGMHWVRQAPGKGLEWVAFIYGAYSGTYYADSVKGR | |
| | | | FTISADTSKNTAYLQMNSLRAEDTAVYYCSRSQSYTYW | |
| | | | SAMDYWGQGTLVTVSS | |
| VVGADGVGK | HLA-A11 | KRAS_G12D_A11_cl18 | DIQMTQSPSSLSASVGDRVTITCRASQDVNTAVAWYQQ | 367 |
| (SEQ ID NO: 24) | | | KPGKAPKLLIYSASFLYSGVPSRFSGSRSGTDFTLTIS | |
| | | | SLQPEDFATYYCQQYDFTPMTFGQGTKVEIKRTGGGSG | |
| | | | GGGSGGGASEVQLVESGGGLVQPGGSLRLSCAASGFNI | |
| | | | SYAKMHWVRQAPGKGLEWVAYLTYWGGYTNYADSVKGR | |
| | | | FTISADTSKNTAYLQMNSLRAEDTAVYYCSRGEYGTYM | |
| | | | DYWGQGTLVTVSS | |
| VVGADGVGK | HLA-A11 | KRAS_G12D_A11_c119 | DIQMTQSPSSLSASVGDRVTITCRASQDVNTAVAWYQQ | 368 |
| (SEQ ID NO: 24) | | | KPGKAPKLLIYSASFLYSGVPSRFSGSRSGTDFTLTIS | |
| | | | SLQPEDFATYYCQQYSSSSPVTFGQGTKVEIKRTGGGS | |
| | | | GGGGSGGGASEVQLVESGGGLVQPGGSLRLSCAASGFN | |
| | | | FSSYVMHWVRQAPGKGLEWVAVVYPDSGGTYYADSVKG | |
| | | | RFTISADTSKNTAYLQMNSLRAEDTAVYYCSRTSSYYA | |
| | | | FDYWGQGTLVTVSS | |

10) KRAS G12V(7-16)-A11

| | | | | |
|---|---|---|---|---|
| VVVGAVGVGK | HLA-A11 | KRAS_G12V_A11_V3 | DIQMTQSPSSLSASVGDRVTITCRASQDVNTAVAWYQQ | 369 |
| (SEQ ID NO: 22) | | | KPGKAPKLLIYSASFLYSGVPSRFSGSRSGTDFTLTIS | |
| | | | SLQPEDFATYYCQQSSYTPITFGQGTKVEIKRTGGGSG | |
| | | | GGGSGGGASEVQLVESGGGLVQPGGSLRLSCAASGFNI | |
| | | | SQGGIHWVRQAPGKGLEWVAYVYPGGGQTNYADSVKGR | |
| | | | FTISADTSKNTAYLQMNSLRAEDTAVYYCSRGYDYSAF | |
| | | | DYWGQGTLVTVSS | |
| VVVGAVGVGK | HLA-A11 | KRAS_G12_VA11_V9 | DIQMTQSPSSLSASVGDRVTITCRASQDVNTAVAWYQQ | 370 |
| (SEQ ID NO: 22) | | | KPGKAPKLLIYSASFLYSGVPSRFSGSRSGTDFTLTIS | |
| | | | SLQPEDFATYYCQQYAYYPITFGQGTKVEIKRTGGGSG | |
| | | | GGGSGGGASEVQLVESGGGLVQPGGSLRLSCAASGFNI | |
| | | | SSTGMHWVRQAPGKGLEWVAELLGGSGNTNYADSVKGR | |
| | | | FTISADTSKNTAYLQMNSLRAEDTAVYYCSRGLQYSAM | |
| | | | DYWGQGTLVTVSS | |
| VVVGAVGVGK | HLA-A11 | KRAS_G12V_A11V10 | DIQMTQSPSSLSASVGDRVTITCRASQDVNTAVAWYQQ | 371 |
| (SEQ ID NO: 22) | | | KPGKAPKLLIYSASFLYSGVPSRFSGSRSGTDFTLTIS | |
| | | | SLQPEDFATYYCQQYEYYPITFGQGTKVEIKRTGGGSG | |
| | | | GGGSGGGASEVQLVESGGGLVQPGGSLRLSCAASGFNF | |
| | | | FSTVIHWVRQAPGKGLEWVAEIYPWSGSTYYADSVKGR | |
| | | | FTISADTSKNTAYLQMNSLRAEDTAVYYCSRSRSSNYY | |
| | | | FDVWGQGTLVTVSS | |
| VVVGAVGVGK | HLA-A11 | KRAS_G12V_A11_V21 | DIQMTQSPSSLSASVGDRVTITCRASQDVNTAVAWYQQ | 372 |
| (SEQ ID NO: 22) | | | KPGKAPKLLIYSASFLYSGVPSRFSGSRSGTDFTLTIS | |
| | | | SLQPEDFATYYCQQYTYYPITFGQGTKVEIKRTGGGSG | |
| | | | GGGSGGGASEVQLVESGGGLVQPGGSLRLSCAASGFNL | |
| | | | HGYLMHWVRQAPGKGLEWVAFIYPPNGYTYYADSVKGR | |
| | | | FTISADTSKNTAYLQMNSLRAEDTAVYYCSRGVDYAYL | |
| | | | DYWGQGTLVTVSS | |
| VVVGAVGVGK | HLA-A11 | KRAS_G12V_A11_V23 | DIQMTQSPSSLSASVGDRVTITCRASQDVNTAVAWYQQ | 373 |
| (SEQ ID NO: 22) | | | KPGKAPKLLIYSASFLYSGVPSRFSGSRSGTDFTLTIS | |
| | | | SLQPEDFATYYCQQYSYYPITFGQGTKVEIKRTGGGSG | |
| | | | GGGSGGGASEVQLVESGGGLVQPGGSLRLSCAASGFNL | |
| | | | STHVMHWVRQAPGKGLEWVAEFYPYVGYTYYADSVKGR | |
| | | | FTISADTSKNTAYLQMNSLRAEDTAVYYCSRGYRYQYM | |
| | | | DVWGQGTLVTVSS | |
| VVVGAVGVGK | HLA-A11 | KRAS_G12V_A11_V24 | DIQMTQSPSSLSASVGDRVTITCRASQDVNTAVAWYQQ | 374 |
| (SEQ ID NO: 22) | | | KPGKAPKLLIYSASFLYSGVPSRFSGSRSGTDFTLTIS | |
| | | | SLQPEDFATYYCQQSSVEPWTFGQGTKVEIKRTGGGSG | |
| | | | GGGSGGGASEVQLVESGGGLVQPGGSLRLSCAASGFNV | |
| | | | SYYSIHWVRQAPGKGLEWVAYIYPWNDYTYYADSVKGR | |
| | | | FTISADTSKNTAYLQMNSLRAEDTAVYYCSRGSYYSFD | |
| | | | YWGQGTLVTVSS | |

11) CTNNB S45F(41-49)-A3

| | | | | |
|---|---|---|---|---|
| TTAPFLSGK | HLA-A3 | CTNNB_S45F_A3_E10 | DIQMTQSPSSLSASVGDRVTITCRASQDVNTAVAWYQQ | 375 |
| (SEQ ID NO: 26) | | | KPGKAPKLLIYSASFLYSGVPSRFSGSRSGTDFTLTIS | |
| | | | SLQPEDFATYYCQQSYYSPPTFGQGTKVEIKRTGGGSG | |
| | | | GGASEVQLVESGGGLVQPGGSLRLSCAASGFNINNTYI | |
| | | | HWVRQAPGKGLEWVASIYPTDGYTRYADSVKGRFTISA | |
| | | | DTSKNTAYLQMNSLRAEDTAVYYCSRTYYSYYSAMDVW | |
| | | | GQGTLVTVSS | |
| TTAPFLSGK | HLA-A3 | CTNNB_S45_FA3_cl3 | DIQMTQSPSSLSASVGDRVTITCRASQDVNTAVAWYQQ | 376 |
| (SEQ ID NO: 26) | | | KPGKAPKLLIYSAYFLYSGVPSRFSGSRSGTDFTLTIS | |
| | | | SLQPEDFATYYCQQIYTSPITFGQGTKVEIKRTGGGSG | |
| | | | GGGSGGGASEVQLVESGGGLVQPGGSLRLSCAASGFNF | |
| | | | ITTGMHWVRQAPGKGLEWVARIGPGSDYTNYADSVKGR | |
| | | | FTISADTSKNTAYLQMNSLRAEDTAVYYCSRYYYASAL | |
| | | | DYWGQGTLVTVSS | |
| TTAPFLSGK | HLA-A3 | CTNNB_S45_FA3_cl4 | DIQMTQSPSSLSASVGDRVTITCRASQDVNTAVAWYQQ | 377 |
| (SEQ ID NO: 26) | | | KPGKAPKLLIYSASFLYSGVPSRFSGSRSGTDFTLTIS | |
| | | | SLQPEDFATYYCQQRAYFPITFGQGTKVEIKRTGGGSG | |
| | | | GGGSGGGASEVQLVESGGGLVQPGGSLRLSCAASGFNF | |
| | | | SDYGMHWVRQAPGKGLEWVAMLIPASGYTNYADSVKGR | |
| | | | FTISADTSKNTAYLQMNSLRAEDTAVYYCSRGWSYYMD | |
| | | | YWGQGTLVTVSS | |
| TTAPFLSGK | HLA-A3 | CTNNB_S45_FA3 cl7 | DIQMTQSPSSLSASVGDRVTITCRASQDVNTAVAWYQQ | 378 |
| (SEQ ID NO: 26) | | | KPGKAPKLLIYSASFLYSGVPSRFSGSRSGTDFTLTIS | |
| | | | SLQPEDFATYYCQQQYAYTPITFGQGTKVEIKRTGGGS | |
| | | | GGGGSGGGASEVQLVESGGGLVQPGGSLRLSCAASGFN | |
| | | | VWSYGIHWVRQAPGKGLEWVAGVTPDGSYTYYADSVKG | |
| | | | RFTISADTSKNTAYLQMNSLRAEDTAVYYCSRSYGWAM | |
| | | | DYWGQGTLVTVSS | |
| TTAPFLSGK | HLA-A3 | CTNNB_S45_FA3 cl9 | DIQMTQSPSSLSASVGDRVTITCRASQDVNTAVAWYQQ | 379 |
| (SEQ ID NO: 26) | | | KPGKAPKLLIYSAYFLYSGVPSRFSGSRSGTDFTLTIS | |
| | | | SLQPEDFATYYCQQIHYKPLTFGQGTKVEIKRTGGGSG | |
| | | | GGGSGGGASEVQLVESGGGLVQPGGSLRLSCAASGFNV | |
| | | | AWYSIHWVRQAPGKGLEWVAQVYGGSSYTYYADSVKGR | |
| | | | FTISADTSKNTAYLQMNSLRAEDTAVYYCSRDFYSSGM | |
| | | | DYWGQGTLVTVSS | |

12) KRAS G12V(11-19)-B7

| | | | | |
|---|---|---|---|---|
| AVGVGKSAL | HLA-B7 | KRAS_G12VB7_cl1 | DIQMTQSPSSLSASVGDRVTITCRASQDVNTAVAWYQQ | 380 |
| (SEQ ID NO: 11) | | | KPGKAPKLLIYSASFLYSGVPSRFSGSRSGTDFTLTIS | |
| | | | SLQPEDFATYYCQQEWRLPITFGQGTKVEIKRTGGGSG | |
| | | | GGGSGGGASEVQLVESGGGLVQPGGSLRLSCAASGFNV | |
| | | | YGNQIHWVRQAPGKGLEWVARIYPYSGSTYYADSVKGR | |
| | | | FTISADTSKNTAYLQMNSLRAEDTAVYYCSRSAYVAYS | |
| | | | YFDYWGQGTLVTVSS | |
| AVGVGKSAL | HLA-B7 | KRAS_G12VB7_cl2 | DIQMTQSPSSLSASVGDRVTITCRASQDVNTAVAWYQQ | 390 |
| (SEQ ID NO: 11) | | | KPGKAPKLLIYSASFLYSGVPSRFSGSRSGTDFTLTIS | |
| | | | SLQPEDFATYYCQQGTSTPFTFGQGTKVEIKRTGGGSG | |
| | | | GGGSGGGASEVQLVESGGGLVQPGGSLRLSCAASGFNL | |
| | | | SYYGMHWVRQAPGKGLEWVATIYPDSGYTKYADSVKGR | |
| | | | FTISADTSKNTAYLQMNSLRAEDTAVYYCSRAYLYYYL | |
| | | | AYWGQGTLVTVSS | |
| AVGVGKSAL | HLA-B7 | KRAS_G12VB7_cl3 | DIQMTQSPSSLSASVGDRVTITCRASQDVNTAVAWYQQ | 391 |
| (SEQ ID NO: 11) | | | KPGKAPKLLIYSASFLYSGVPSRFSGSRSGTDFTLTIS | |
| | | | SLQPEDFATYYCQQSWRYPMTFGQGTKVEIKRTGGGSG | |
| | | | GGGSGGGASEVQLVESGGGLVQPGGSLRLSCAASGFNI | |
| | | | SRYGMHWVRQAPGKGLEWVAVFYPSSSYTYYADSVKGR | |
| | | | FTISADTSKNTAYLQMNSLRAEDTAVYYCSRKYYEAMD | |
| | | | YWGQGTLVTVSS | |
| AVGVGKSAL | HLA-B7 | KRAS_G12VB7_cl5 | DIQMTQSPSSLSASVGDRVTITCRASQDVNTAVAWYQQ | 392 |
| (SEQ ID NO: 11) | | | KPGKAPKLLIYSASFLYSGVPSRFSGSRSGTDFTLTIS | |
| | | | SLQPEDFATYYCQQSYSYPVTFGHGTKVEIKRTGGGSG | |
| | | | GGGSGGGASEVQLVESGGGLVQPGGSLRLSCAASGFNI | |
| | | | YSSWMHWVRQAPGKGLEWVAYFQPYSGYTKYADSVKGR | |
| | | | FTISADTSKNTAYLQMNSLRAEDTAVYYCSREYTYYFD | |
| | | | YWGQGTLVTVSS | |
| AVGVGKSAL | HLA-B7 | KRAS_G12VB7_cl6 | DIQMTQSPSSLSASVGDRVTITCRASQDVNTAVAWYQQ | 393 |
| (SEQ ID NO: 11) | | | KPGKAPKLLIYSASFLYSGVPSRFSGSRSGTDFTLTIS | |
| | | | SLQPEDFATYYCQQGWLYSPFTFGQGTKVEIKRTGGGS | |
| | | | GGGGSGGGASEVQLVESGGGLVQPGGSLRLSCAASGFN | |
| | | | ISGYGMHWVRQAPGKGLEWVARVYGGSGYTYYADSVKG | |
| | | | RFTISADTSKNTAYLQMNSLRAEDTAVYYCSRAHSSYY | |
| | | | VDYWGQGTLVTVSS | |

13) H/K/N RAS Q61H(55-64)-A1

| | | | | |
|---|---|---|---|---|
| ILDTAGHEEY | HLA-A1 | H/K/N RAS | DIQMTQSPSSLSASVGDRVTITCRASQDVNTAVAWYQQ | 394 |
| (SEQ ID NO: 28) | | Q61H_A1_cl0 | KPGKAPKLLIYSASFLYSGVPSRFSGSRSGTDFTLTIS | |
| | | | SLQPEDFATYYCQQGYFYYPNTFGQGTKVEIKRTGGGS | |
| | | | GGGGSGGGASEVQLVESGGGLVQPGGSLRLSCAASGFN | |
| | | | IGYYGMHWVRQAPGKGLEWVATVYPGGGYTSYADSVKG | |
| | | | RFTISADTSKNTAYLQMNSLRAEDTAVYYCSRYYYYGF | |
| | | | DYWGQGTLVTVSS | |

14) H/K/N RAS Q61K(55-64)-A1

| | | | | |
|---|---|---|---|---|
| ILDTAGKEEY | HLA-A1 | H/K/N RAS | DIQMTQSPSSLSASVGDRVTITCRASQDVNTAVAWYQQ | 395 |
| (SEQ ID NO: 30) | | Q61K_A1_cl6 | KPGKAPKLLIYSASFLYSGVPSRFSGSRSGTDFTLTIS | |
| | | | SLQPEDFATYYCQQHYYSPVTFGQGTKVEIKRTGGGSG | |
| | | | GGGSGGGASEVQLVESGGGLVQPGGSLRLSCAASGFNI | |
| | | | FYQDMHWVRQAPGKGLEWVAMIYPDYDYTYYADSVKGR | |
| | | | FTISADTSKNTAYLQMNSLRAEDTAVYYCSRTYSVYMD | |
| | | | YWGQGTLVTVSS | |

15) H/K/N RAS Q61L(55-64)-A1

| | | | | |
|---|---|---|---|---|
| ILDTAGLEEY | HLA-A1 | H/K/N RAS | DIQMTQSPSSLSASVGDRVTITCRASQDVNTAVAWYQQ | 396 |
| (SEQ ID NO: 31) | | Q61K_A1_cl8 | KPGKAPKLLIYSASFLYSGVPSRFSGSRSGTDFTLTIS | |
| | | | SLQPEDFATYYCQQYAYAPFTFGQGTKVEIKRTGGGSG | |
| | | | GGGSGGGASEVQLVESGGGLVQPGGSLRLSCAASGFNV | |
| | | | SYSMIHWVRQAPGKGLEWVARVWGDGGVTYYADSVKGR | |
| | | | FTISADTSKNTAYLQMNSLRAEDTAVYYCSRGSYYAFD | |
| | | | YWGQGTLVTVSS | |

16) H/K/N RAS Q61R(55-64)-A1

| | | | | |
|---|---|---|---|---|
| ILDTAGREEY | HLA-A1 | H/R/N RAS | DIQMTQSPSSLSASVGDRVTITCRASQDVNTAVAWYQQ | 397 |
| (SEQ ID NO: 32) | | Q61R_A1_cl16 | KPGKAPKLLIYSASFLYSGVPSRFSGSRSGTDFTLTIS | |
| | | | SLQPEDFATYYCQQAHMIPITFGQGTKVEIKRTGGGSG | |
| | | | GGGSGGGASEVQLVESGGGLVQPGGSLRLSCAASGFNF | |
| | | | SFPGMEIWVRQAPGKGLEWVAWFVGYDGYTYYADSVKG | |
| | | | RFTISADTSKNTAYLQMNSLRAEDTAVYYCSRDYYSFS | |
| | | | MDYWGQGTLVTVSS | |
| ILDTAGREEY | HLA-A1 | H/R/N RAS | DIQMTQSPSSLSASVGDRVTITCRASQDVNTAVAWYQQ | 398 |
| (SEQ ID NO: 32) | | Q61R_A1_cl17 | KPGKAPKLLIYSASFLYSGVPSRFSGSRSGTDFTLTIS | |
| | | | SLQPEDFATYYCQQSVYDPITFGQGTKVEIKRTGGGSG | |
| | | | GGGSGGGASEVQLVESGGGLVQPGGSLRLSCAASGFNI | |
| | | | SGSWIHWVRQAPGKGLEWVAWLYPDSDYTYYADSVKGR | |
| | | | FTISADTSKNTAYLQMNSLRAEDTAVYYCSRAHTYAFD | |
| | | | YWGQGTLVTVSS | |
| ILDTAGREEY | HLA-A1 | H/R/N RAS | DIQMTQSPSSLSASVGDRVTITCRASQDVNTAVAWYQQ | 399 |
| (SEQ ID NO: 32) | | Q61R_A1_cl18 | KPGKAPKLLIYSASFLYSGVPSRFSGSRSGTDFTLTIS | |
| | | | SLQPEDFATYYCQQSYTSPLTFGQGTKVEIKRTGGGSG | |
| | | | GGGSGGGASEVQLVESGGGLVQPGGSLRLSCAASGFNI | |
| | | | YYGVMHWVRQAPGKGLEWVAMIYPDSSWTYYADSVKGR | |
| | | | FTISADTSKNTAYLQMNSLRAEDTAVYYCSRDQDFHYM | |
| | | | NYYLSYALDYWGQGTLVTVSS | |
| ILDTAGREEY | HLA-A1 | H/R/N RAS | DIQMTQSPSSLSASVGDRVTITCRASQDVNTAVAWYQQ | 400 |
| (SEQ ID NO: 32) | | Q61R_A1_cl19 | KPGKAPKLLIYSASFLYSGVPSRFSGSRSGTDFTLTIS | |
| | | | SLQPEDFATYYCQQGQYSPFTFGQGTKVEIKRTGGGSG | |
| | | | GGGSGGGASEVQLVESGGGLVQPGGSLRLSCAASGFNV | |
| | | | SYEYMHWVRQAPGKGLEWVAEIYGGSDNTYYADSVKGR | |
| | | | FTISADTSKNTAYLQMNSLRAEDTAVYYCSRPLGSYFD | |
| | | | YWGQGTLVTVSS | |
| ILDTAGREEY | HLA-A1 | H/R/N RAS | DIQMTQSPSSLSASVGDRVTITCRASQDVNTAVAWYQQ | 401 |
| (SEQ ID NO: 32) | | Q61R_A1_cl22 | KPGKAPKLLIYSASFLYSGVPSRFSGSRSGTDFTLTIS | |
| | | | SLQPEDFATYYCQQYWYLPTTFGQGTKVEIKRTGGGSG | |
| | | | GGGSGGGASEVQLVESGGGLVQPGGSLRLSCAASGFNI | |
| | | | SWYDIHWVRQAPGKGLEWVADIEPSVGYTYYADSVKGR | |
| | | | FTISADTSKNTAYLQMNSLRAEDTAVYYCSRSYPYYYF | |
| | | | DYWGQGTLVTVSS | |

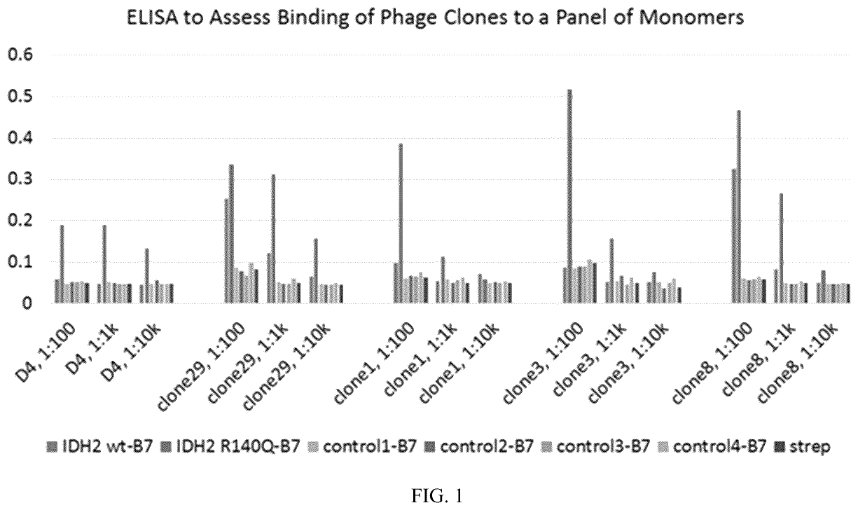

Representative ELISA data for a scFvs that specifically recognized an IDH2 peptide containing the R140Q mutation in complex with HLA-B7 (SPNGTIQNIL; SEQ ID NO: 1) are shown in FIG. 1. The scFvs did not recognize the wt version of the peptide of interest in complex with the same HLA allele. The scFvs did not recognize other control peptides in complex with the HLA allele when tested for binding to a monomer-coated ELISA plate.

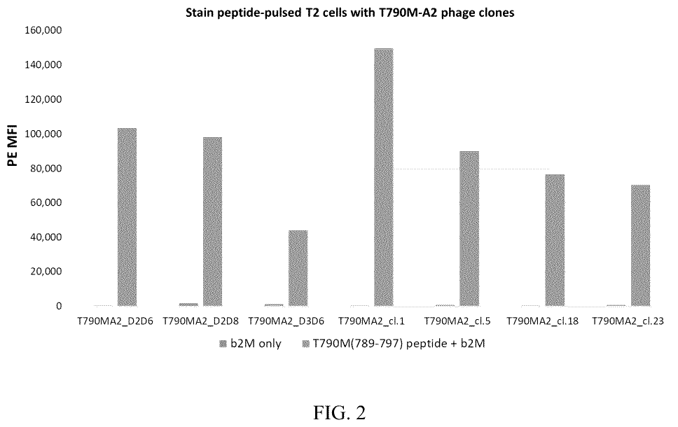

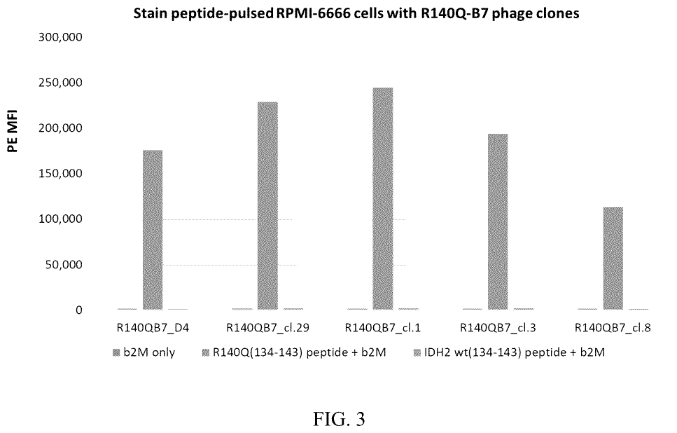

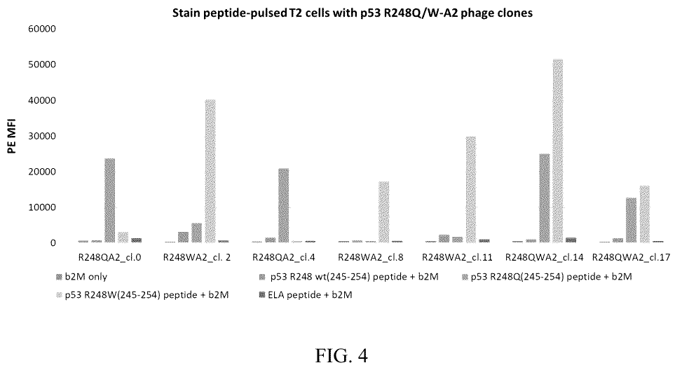

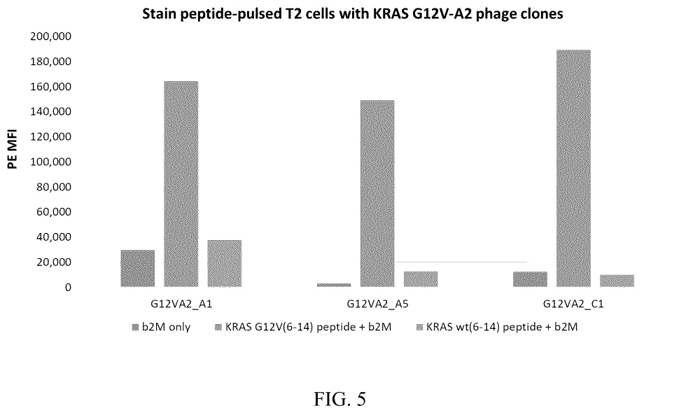

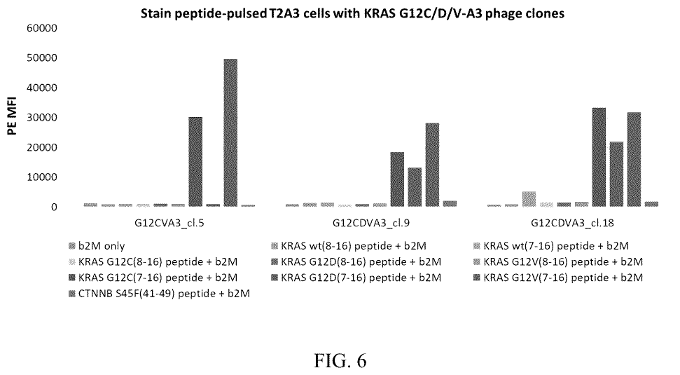

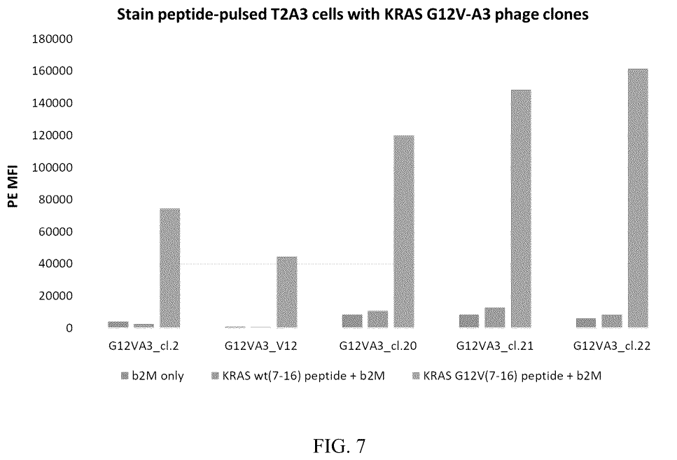

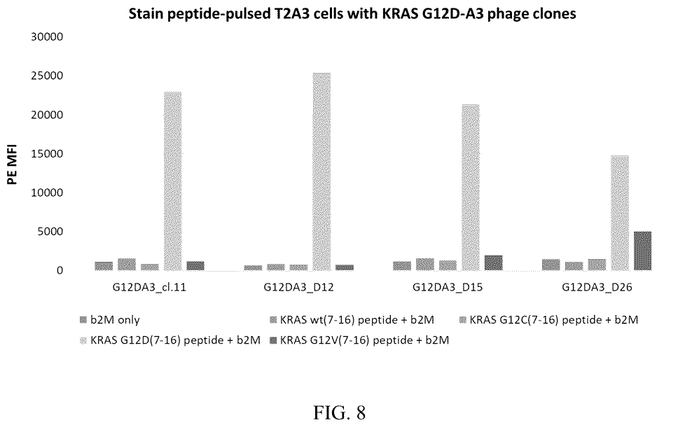

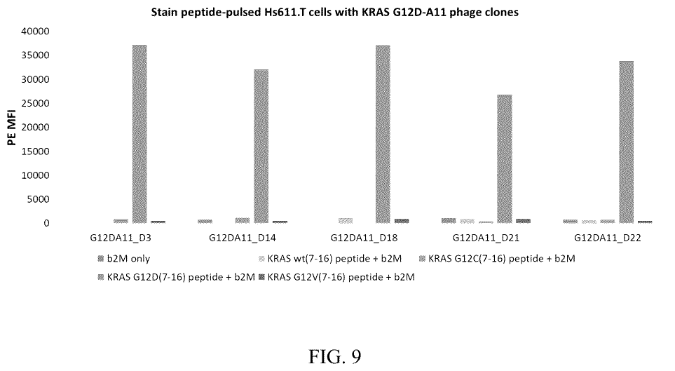

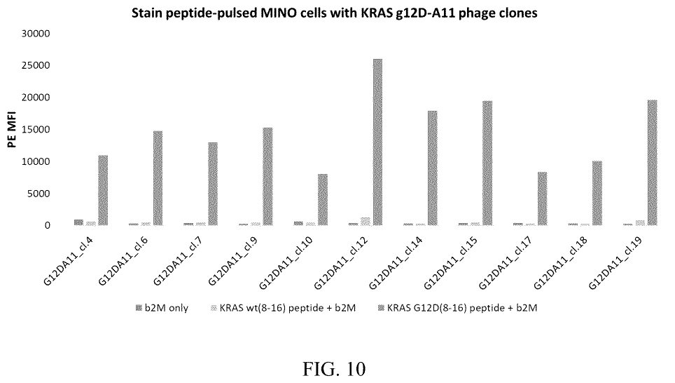

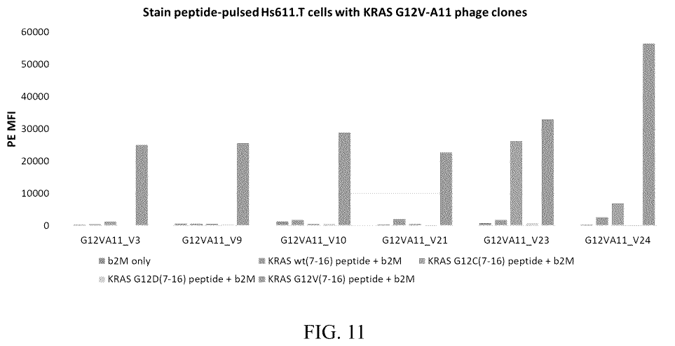

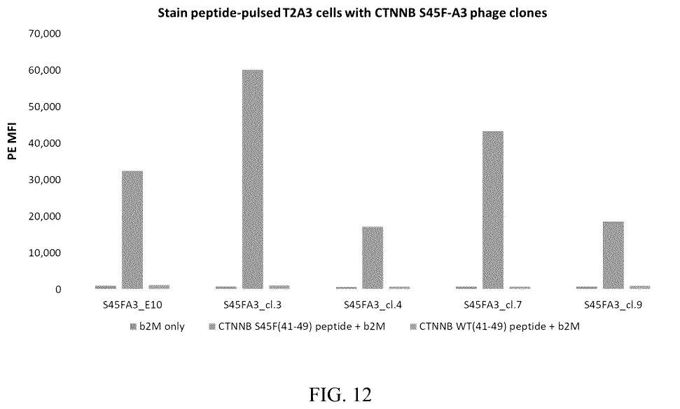

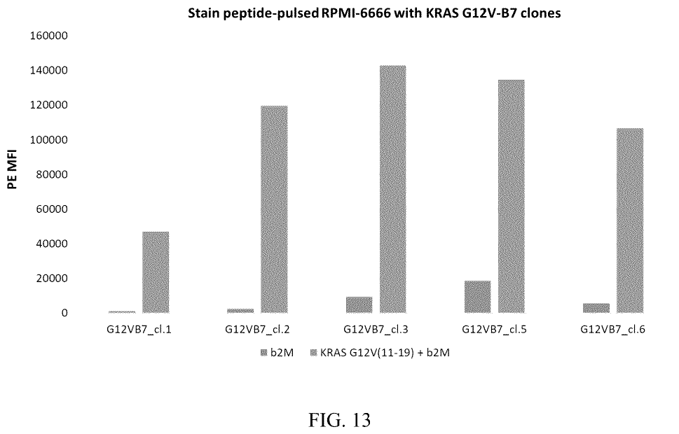

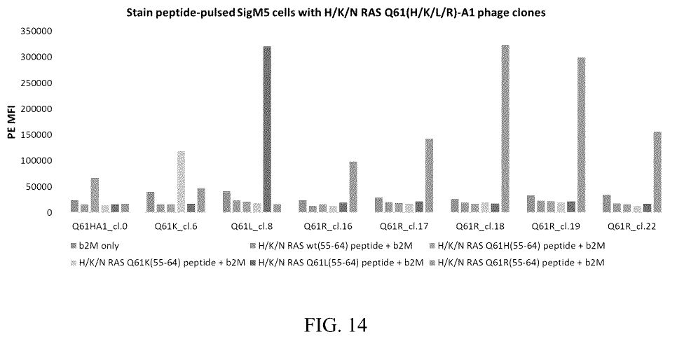

Further flow cytometry using showed that MANAbody scFv clones specifically stain the HLA allele-matched cell lines when these cells are pulsed with the mutant peptide, but not the wt peptide or other control peptides (FIGS. 2-14).

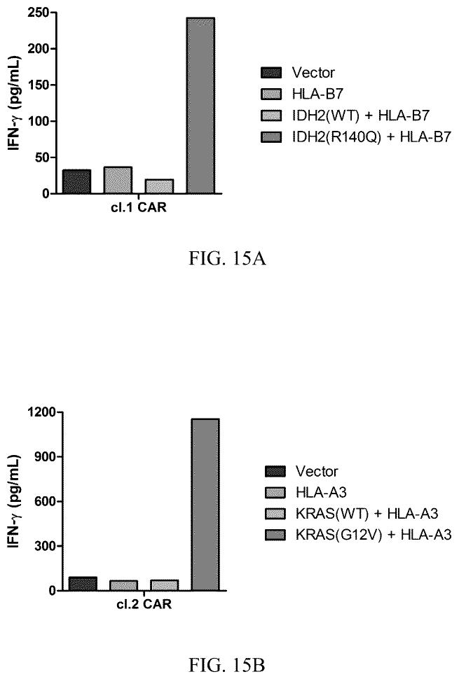

To demonstrate that MANAbody clones can be utilized as a therapeutic modality, selected MANAbody clones were engineered into CAR-T cells. Chimeric antigen receptor (CAR) T cells (CARTs) capable of recognizing and killing cells expressing oncogenic mutation-containing peptides in the context of HLA molecules via their endogenous processing and presentation machinery were engineered. Specifically, CARTs targeting a mutant KRAS G12V peptide presented in the context of HLA-A3 were engineered, and CARTs targeting a mutant IDH2 R140Q peptide presented in the context of HLA-B7 were engineered. MANAbody scFvs targeting either mutant peptide were grafted onto a 3^{rd} Generation CAR construct, and CAR receptors were expressed in CD3+ T cells by mRNA electroporation. CAR-T cells were subsequently co-cultured with COS-7 cells co-transfected with plasmids encoding KRAS/IDH2 mutant and wt proteins in combination with their respective HLA. As T cells, including CAR-T cells, produce cytokines following activation by cognate antigen on target cells, the release of IFNγ in the co-culture media supernatant was measured by ELISA. Only when COS-7 cells were co-transfected with the mutant and cognate HLA plasmids was there significant release of IFNγ over background (FIG. 15). CAR-T cells co-cultured with COS-7 cells co-transfected with the wt and cognate HLA released only background levels of IFNγ. Together, these findings suggest that CAR-T cells expressing MANAbody clones can target tumor cells expressing MANAs presented in the context of HLA molecules.

To demonstrate that MANAbody clones can be utilized as a therapeutic modality, selected MANAbody clones were engineered into bispecific antibodies. A bispecific antibody having one antibody-fragment binding to a target cancer cell and having one antibody-fragment binding to a CD3 protein on the T cell surface was engineered. There are a number of different anti-CD3 scFv clones targeting human CD3 epsilon, delta, and/or gamma molecules. Examples of such clones are listed in Table 4.

Bispecific antibodies having one antibody-fragment binding to a mutant KRAS G12V peptide presented in the context of HLA-A3 and having one antibody-fragment binding to a CD3 protein on the T cell surface were engineered. Specifically, bispecific antibodies targeting a mutant KRAS G12V peptide presented in the context of HLA-A3 and CD3 were engineered, and bispecific antibodies targeting a mutant IDH2 R140Q peptide presented in the context of HLA-B7 and CD3 were engineered.

TABLE 4
Anti-human CD3 scFv sequences.

| | | SEQ |
|---|---|---|
| Clone Name | Clone scFv Sequence | ID NO: |
| humanized | DIQMTQSPSSLSASVGDRVTITCRASQDIRNYLNWYQQKPGKAPKLLIYYTSRLESGVPSRFSGSGSGTDYT | 404 |
| UCHT1 | LTISSLQPEDFATYYCQQGNTLPWTFGQGTKVEIKGGGGSGGGGSGGGGSEVQLVESGGGLVQPGGSLRLSC | |
| (hUCHT1v9) | AASGYSFTGYTMNWVRQAPGKGLEWVALINPYKGVSTYNQKFKDRFTISVDKSKNTAYLQMNSLRAEDTAVY | |
| | YCARSGYYGDSDWYFDVWGQGTLVTVSS | |
| murine UCHT1 | DIQMTQTTSSLSASLGDRVTISCRASQDIRNYLNWYQQKPDGTVKLLIYYTSRLHSGVPSKFSGSGSGTDYS | 405 |
| (mUCHT1) | LTISNLEQEDIATYFCQQGNTLPWTFAGGTKLEIKGGGGSGGGGSGGGGSEVQLQQSGPELVKPGASMKISC | |
| | KASGYSFTGYTMNWVKQSHGKNLEWMGLINPYKGVSTYNQKFKDKATLTVDKSSSTAYMELLSLTSEDSAVY | |
| | YCARSGYYGDSDWYFDVWGAGTTVTVSS | |
| diL2K | DIVLTQSPATLSLSPGERATLSCRASQSVSYMNWYQQKPGKAPKRWIYDTSKVASGVPARFSGSGSGTDYSL | 406 |
| | TINSLEAEDAATYYCQQWSSNPLTFGGGTKVEIKGGGGSGGGGSGGGGSDVQLVQSGAEVKKPGASVKVSCK | |
| | ASGYTFTRYTMHWVRQAPGQGLEWIGYINPSRGYTNYADSVKGRFTITTDKSTSTAYMELSSLRSEDTATYY | |
| | CARYYDDHYCLDYWGQGTTVTVSS | |
| hXR32 | QAVVTQEPSLTVSPGGTVTLTCRSSTGAVTTSNYANWVQQKPGQAPRGLIGGTNKRAPWTPARFSGSLLGGK | 407 |
| | AALTITGAQAEDEADYYCALWYSNLWVFGGGTKLTVLGGGGSGGGGSGGGGSEVQLVESGGGLVQPGGSLRL | |
| | SCAASGFTFNTYAMNWVRQAPGKGLEWVARIRSKYNNYATYYADSVKDRFTISRDDSKNSLYLQMNSLKTED | |
| | TAVYYCVRHGNFGNSYVSWFAYWGQGTLVTVSS | |
| L2K-07 | DIQLTQSPAIMSASPGEKVTMTCRASSSVSYMNWYQQKSGTSPKRWIYDTSKVASGVPYRFSGSGSGTSYSL | 408 |
| | TISSMEAEDAATYYCQQWSSNPLTFGAGTKLELKGGGGSGGGGSGGGGSDIKLQQSGAELARPGASVKMSCK | |
| | TSGYTFTRYTMHWVKQRPGQGLEWIGYINPSRGYTNYNQKFKDKATLTTDKSSSTAYMQLSSLTSEDSAVYY | |
| | CARYYDDHYCLDYWGQGTTLTVSS | |
| OKT3 | QIVLTQSPAIMSASPGEKVTMTCSASSSVSYMNWYQQKSGTSPKRWIYDTSKLASGVPAHFRGSGSGTSYSL | 409 |
| | TISGMEAEDAATYYCQQWSSNPFTFGSGTKLEINGGGGSGGGGSGGGGSQVQLQQSGAELARPGASVKMSCK | |
| | ASGYTFTRYTMHWVKQRPGQGLEWIGYINPSRGYTNYNQKFKDKATLTTDKSSSTAYMQLSSLTSEDSAVYY | |
| | CARYYDDHYCLDYWGQGTTLTVSS | |
| PSMA-CD3 | QTVVTQEPSLTVSPGGTVTLTCGSSTGAVTSGNYPNWVQQKPGQAPRGLIGGTKFLAPGTPARFSGSLLGGK | 410 |
| | AALTLSGVQPEDEAEYYCVLWYSNRWVFGGGTKLTVLGGGGSGGGGSGGGGSEVQLVESGGGLVQPGGSLKL | |
| | SCAASGFTFNKYAMNWVRQAPGKGLEWVARIRSKYNNYATYYADSVKDRFTISRDDSKNTAYLQMNNLKTED | |
| | TAVYYCVRHGNFGNSYISYWAYWGQGTLVTVSS | |
| 28F11 | EIVLTQSPATLSLSPGERATLSCRASQSVSSYLAWYQQKPGQAPRLLIYDASNRATGIPARFSGSGSGTDFT | 411 |
| | LTISSLEPEDFAVYYCQQRSNWPPLTFGGGTKVEIKGGGGSGGGGSGGGGSQVQLVESGGGVVQPGRSLRLS | |
| | CAASGFKFSGYGMHWVRQAPGKGLEWVAVIWYDGSKKYYVDSVKGRFTISRDNSKNTLYLQMNSLRAEDTAV | |
| | YYCARQMGYWHFDLWGRGTLVTVSS | |
| 27H5-VL1 | EIVLTQSPRTLSLSPGERATLSCRASQSVSSSYLAWYQQKPGQAPRLLIYGASSRATGIPDRFSGSGSGTDF | 412 |
| | TLTISRLDPEDFAVYYCQQYGSSPITFGQGTRLEIKGGGGSGGGGSGGGGSQVQLVESGGGVVQPGRSLRLS | |
| | CAASGFTFRSYGMHWVRQAPGKGLEWVAIIWYDGSKKNYADSVKGRFTISRDNSKNTLYLQMNSLRAEDTAV | |
| | YYCARGTGYNWFDPWGQGTLVTVSS | |
| 27H5-VL2 | DILMTQSPSSLSASVGDRVTITCRASQGISSALAWYQQKPGKAPKLLIYYASSLQSGVPSRFSGSGSGTDYT | 413 |
| | LTISSLQPEDFATYYCQQYYSTLTFGGGTKVEIKGGGGSGGGGSGGGGSQVQLVESGGGVVQPGRSLRLSCA | |
| | ASGFTFRSYGMHWVRQAPGKGLEWVAIIWYDGSKKNYADSVKGRFTISRDNSKNTLYLQMNSLRAEDTAVYY | |
| | CARGTGYNWFDPWGQGTLVTVSS | |
| 23F10 | EIVLTQSPATLSLSPGERATLSCRASQSVSSYLAWYQQKPGQAPRLLIYDASNRATGIPARFSGSGSGTDFT | 414 |
| | LTISSLEPEDFAVYYCQQRSNWPPLTFGGGTKVEIKGGGGSGGGGSGGGGSQVQLVQSGGGVVQSGRSLRLS | |
| | CAASGFKFSGYGMHWVRQAPGKGLEWVAVIWYDGSKKYYVDSVKGRFTISRDNSKNTLYLQMNSLRGEDTAV | |
| | YYCARQMGYWHFDLWGRGTLVTVSS | |
| 15C3-VL1 | EIVLTQSPATLSLSPGERATLSCRASQSVSSYLAWYQQKPGQAPRLLIYDASNRATGIPARFSGSGSGTDFT | 415 |
| | LTISSLEPEDFAVYYCQQRSNWPWTFGQGTKVEIKGGGGSGGGGSGGGGSQVQLVQSGGGVVQPGRSLRLSC | |
| | VASGFTFSSYGMHWVRQAPGKGLEWVAAIWYNGRKQDYADSVKGRFTISRDNSKNTLYLQMNSLRAEDTAVY | |
| | YCTRGTGYNWFDPWGQGTLVTVSS | |
| 15C3-VL2 | AIQLTQSPSSLSASVGDRVTITCRASQGISSALAWYQQKPGKAPKLLIYDASSLESGVPSRFSGSGSGTDFT | 416 |
| | LTISSLQPEDFATYYCQQFNSYPITFGQGTRLEIKGGGGSGGGGSGGGGSQVQLVQSGGGVVQPGRSLRLSC | |
| | VASGFTFSSYGMHWVRQAPGKGLEWVAAIWYNGRKQDYADSVKGRFTISRDNSKNTLYLQMNSLRAEDTAVY | |
| | YCTRGTGYNWFDPWGQGTLVTVSS | |
| hu 12F6 | QIVLSQSPAILSASPGEKVTMTCRASSSVSYMHWYQQKPGSSPKPWIYATSNLASGVPARFSGSGSGTSYSL | 417 |
| | TISRVEAEDAATYYCQQWSSNPPTFGGGTKLETKRGGGGSGGGGSGGGGSQVQLQQSGAELARPGASVKMSC | |
| | KASGYTFTSYTMHWVKQRPGQGLEWIGYINPSSGYTKYNQKFKDKATLTADKSSSTAYMQLSSLTSEDSAVY | |
| | YCARWQDYDVYFDYWGQGTTLTVSS | |

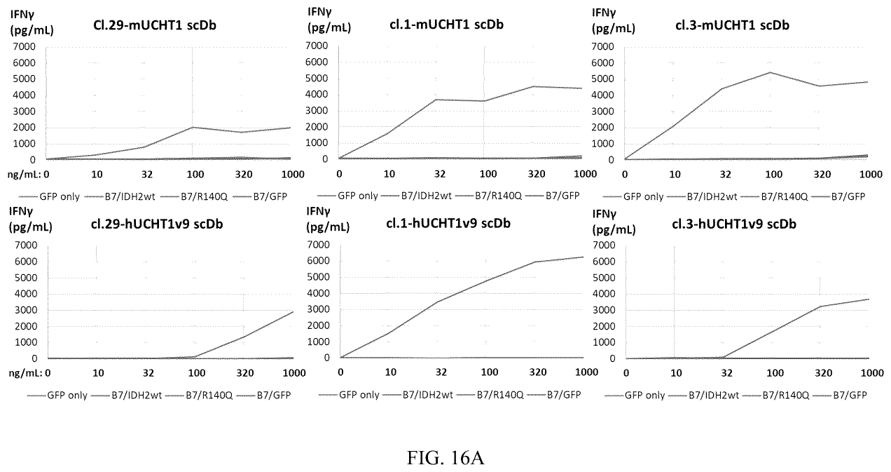

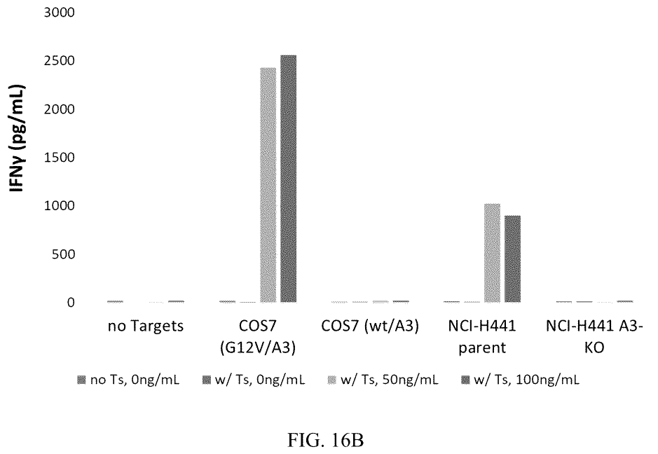

Representative scDb co-culture results are shown in FIG. 16A for three IDH2 R140Q HLA-B7 MANAbody scFv clones combined with two different anti-CD3 scFv clones. T cells were co-cultured with COS-7 cells co-transfected with plasmids encoding HLA-B7, full-length IDH2 variants, and/or GFP in the presence of the specified concentration of scDb. As a read out of T cell activation by cognate antigen on target cells, the release of IFNγ in the co-culture media supernatant was measured by ELISA. Only when COS-7 cells were co-transfected with HLA-B7 and mutant IDH2 R140Q plasmids was there significant T cell release of IFNγ over background, with the level of IFNγ dependent on the concentration of scDb included in the well. T cells co-cultured with COS-7 cells co-transfected with HLA-B7 and wt IDH2 released only background levels of IFNγ. Representative scDb co-culture results are shown in FIG. 16B for a KRAS G12V HLA-A3 MANAbody scFv clone combined with an anti-CD3 clone into a single chain diabody. In this co-culture, the single chain diabody was tested at concentrations of 0, 50, and 100 ng/mL. Only when COS-7 cells were co-transfected with HLA-A3 and mutant KRAS G12V plasmids was there significant T cell release of IFNγ over background. T cells co-cultured with COS-7 cells co-transfected with HLA-A3 and wt KRAS released only background levels of IFNγ, similar to the levels of IFNγ seen in no T cell, no target cell, and no scDb wells. An endogenous KRAS G12V HLA-A3 positive cell line NCI-H441 as a target cell line along with its isogenic HLA-A3 knockout control. IFNγ release was only seen against the parental NCI-H441 cell line but not the HLA-A3 knockout NCI-H441. Together, these findings suggest that bispecific antibodies containing MANAbody clones that target tumor cells expressing MANAs presented in the context of HLA molecules.

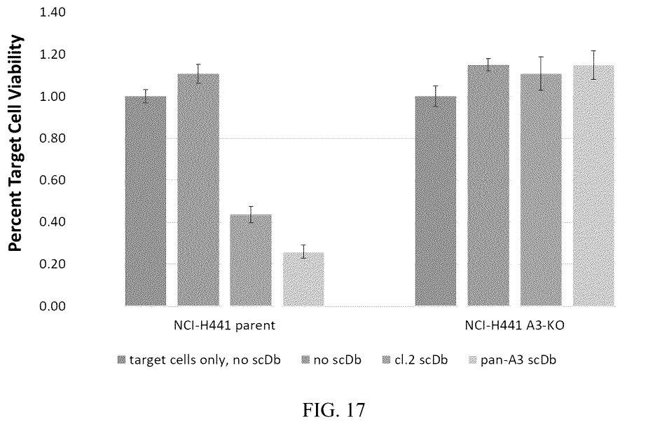

To evaluate the efficacy of using MANAbody clones as a therapeutic modality, target cell viability of a KRAS G12V HLA-A3 single-chain diabody was assayed using PROMEGA® CellTiter-Glo® reagent (FIG. 17). CellTiter-Glo® measures ATP concentration in a well, which is proportional to the number of viable cells. Percent target cell viability was measured by subtracting the CellTiter-Glo® value from T cell only wells and normalizing to target cell only wells. Only when NCI-H441 parent cells were incubated with T cells in the presence of the KRAS G12V-A3 scDb or a pan-HLA-A3 scDb positive control, was there significant target cell death. No target cell death was observed in the absence of scDb or among the NCI-H441 HLA-A3 knockout wells.

Together, these findings demonstrate that MANAbodies can be used to redirect and activate T cells to kill tumor cells expressing particular mutant protein and HLA allele pairs (e.g., IDH2 R140Q with HLA-B7 and KRAS G12V with HLA-A3).

### Materials and Methods

### Cells and Cell Lines.

RPMI-6666 cells (ATCC, Manassas, VA) was cultured in RPMI-1640 (ATCC) with 20% FBS (GE Hyclone, Logan, Utah, USA), and 1% penicillin streptomycin (Life Technologies). T2 cells (ATCC) and MINO cells (ATCC) were cultured in RPMI-1640 (ATCC) with 10% FBS (GE Hyclone), and 1% penicillin streptomycin (Thermo Fisher). T2A3 cells (gifted from Dr. Eric Lutz) were cultured in RPMI-1640 (ATCC) with 10% FBS (GE Hyclone), 1% penicillin streptomycin (Thermo Fisher), 0.1 mM MEM Non-Essential Amino Acids (NEAA, Thermo Fisher), and 500 μg/mL geneticin (Thermo Fisher). SigM5 cells (DSMZ, Brunswick, Germany) were cultured in Iscove's MDM (ATCC) with 20% FBS (GE Hyclone), and 1% penicillin streptomycin (Thermo Fisher). Hs611.T cells (ATCC) was cultured in Dulbecco's Modified Eagle's Medium (ATCC) with 10% FBS (GE Hyclone), and 1% penicillin streptomycin (Thermo Fisher). NCI-H441 cells (ATCC) and COS-7 cells (ATCC) was cultured in McCoy's 5A (Modified) Medium (Thermo Fisher) with 10% FBS (GE Hyclone), and 1% penicillin streptomycin (Thermo Fisher). COS-7 cells (ATCC, CRL-1651™) were cultured in DMEM (high glucose, pyruvate; Thermo Fisher) with 10% FBS (GE Hyclone), and 1% Penicillin-Streptomycin (Thermo Fisher). 293FT cells (Thermo Fisher) were cultured in high-glucose D-MEM (Thermo Fisher), with 10% FBS (GE Hyclone), 0.1 mM MEM Non-Essential Amino Acids (NEAA, Thermo Fisher), 6 mM L-glutamine (Thermo Fisher), 1 mM MEM Sodium Pyruvate (Thermo Fisher), 500 μg/ml geneticin (Thermo Fisher), and 1% Penicillin-Streptomycin (Thermo Fisher). All cell lines were maintained at 37° C. under 5% CO₂.

PBMCs were obtained by Ficoll-Paque PLUS (GE Healthcare) gradient centrifugation of whole blood from healthy volunteer donors. CD3+ cells were positively selected with CD3 MicroBeads (Miltenyi Biotec) from PBMCs, and were activated and expanded with Dynabeads® Human T-Activator CD3/CD28 (Life Technologies). Unless otherwise noted, primary CD3+ T cells were cultured in RPMI-1640 (ATCC) with 10% FBS (GE Hyclone), 1% Penicillin-Streptomycin (Life Technologies), and 100 IU/mL recombinant human interleukin-2 (Proleukin®) at 37° C. under 5% CO₂.

### Phage Display Library Construction.

For the 1st generation phage library, oligonucleotides were synthesized at DNA 2.0 (Menlo Park, CA) using mixed and split pool degenerate oligonucleotide syntheses. For the 2nd generation phage library, oligonucleotides were synthesized at GeneArt (Thermo Fisher, Halethorpe, MD) using trinucleotide mutagenesis (TRIM) technology. For both libraries, the oligonucleotides were incorporated into the pADL-10b phagemid (Antibody Design Labs, San Diego, CA). This phagemid contains an F1 origin, a transcriptional repressor to limit uninduced expression, a lac operator, and a lac repressor. The scFv was synthesized with a pelB periplasmic secretion signal and was subcloned downstream of the lac operator. For the 1st generation library, a myc epitope tag followed by a TEV protease cleavage recognition sequence was placed immediately downstream of the variable heavy chain, while in the 2nd generation library, the scFv was followed by a FLAG tag. Following the scFv, tag, and cleavage site, was the full length, in-frame M13 pIII coat protein sequence.

To transform the phagemid DNA into bacteria, 10-20 ng of the ligation product was mixed on ice with 10 μL of electrocompetent SS320 cells (Lucigen, Middleton, WI) and 14 μL of double-distilled water. This mixture was electroporated using a Gene Pulser electroporation system (Bio-Rad, Hercules, CA) and allowed to recover in Recovery Media (Lucigen) for 60 min at 37° C. Cells transformed with 60 ng of ligation product were pooled and plated on a 24-cm×24-cm plate containing 2×YT medium supplemented with carbenicillin (100 μg/mL) and 2% glucose. Cells were grown at 37° C. for 6 hours and placed at 4° C. overnight. To determine the transformation efficiency for each series of electroporations, aliquots were taken and titered by serial dilution. Cells grown on plates were scraped into 850 mL of 2×YT medium with carbenicillin (100 μg/mL) plus 2% glucose for a final OD600 of 5-15. Two mL of the 850 mL culture were taken and diluted ˜1:200 to reach a final OD600 of 0.05-0.07. To the remaining culture, 150 mL of sterile glycerol were added before snap freezing to produce glycerol stocks. The diluted bacteria were grown to an OD600 of 0.2-0.4, infected with M13K07 Helper phage (Antibody Design Labs, San Diego, CA) and allowed to shake at 37° C. for 1 hour. The culture was centrifuged and the cells were resuspended in 2×YT medium with carbenicillin (100 μg/mL) and kanamycin (50 g/mL) and grown overnight at 30° C. for phage production. The following morning, the bacterial culture was aliquoted into 50 mL Falcon tubes and pelleted twice at high speed to obtain clarified supernatant. The phage-laden supernatant was precipitated on ice for 40 min with a 20% PEG-8000/2.5M NaCl solution at a 4:1 ratio of PEG/NaCl to supernatant. After precipitation, phage was centrifuged at 12,000 g for 40 minutes and resuspended in a 1 mL vol 1×TBS, 2 mM EDTA. Phage from multiple tubes was pooled, re-precipitated, and resuspended to an average titer of 1×10¹³ cfu/mL. For the 1st generation library, the total number of transformants obtained was 5.5×10⁹. For the 2nd generation library, the total number of transformants obtained was 3.6×10¹⁰. Each library was aliquoted and stored in 15% glycerol at −80° C.

### Next-Generation Sequencing of the Complete Phage Library.

DNA from the libraries was amplified using primers that flank the CDR-H3 region. The sequences at the 5′-ends of these primers incorporated molecular barcodes to facilitate unambiguous enumeration of distinct phage sequences. The protocols for PCR-amplification and sequencing are described in Kinde et al. Sequences processed and translated using a custom SQL database and both the nucleotide sequences and amino acid translations were analyzed using Microsoft Excel.

### Peptides and HLA-Monomers.

Mutant, wt, and control peptides (listed in Table 1) were predicted to bind to HLA alleles using NetMHC version 4.0. All peptides were synthesized at a purity of >90% by Peptide 2.0 (Chantilly, VA). Peptides were resuspended in DMSO or DMF at 10 mg/mL and stored at −20° C. HLA monomers were synthesized by refolding recombinant HLA with peptide and beta-2 microglobulin, purified by gel-filtration, and biotinylated (Fred Hutchinson Immune Monitoring Lab, Seattle, WA). Monomers were confirmed to be folded prior to selection by performing an ELISA using W6/32 antibody (BioLegend, San Diego, CA).

### Selection for Phage Binding to Mutant Peptide-HLA Monomers.

Biotinylated monomers containing HLA and beta-2-microglobulin proteins were conjugated to MyOne™ T1 streptavidin magnetic beads (Life Technologies, Carlsbad, CA). The biotinylated monomers were incubated with 30 μL of MyOne™ T1 beads (per 1 μg of monomer) in blocking buffer (PBS, 0.5% BSA, 0.1% Na-azide) for 1 hour at room temperature (RT). After the initial incubation, the complexes were washed 3 times with 1 ml blocking buffer and resuspended in 1 ml blocking buffer.

### Enrichment Phase.

In the enrichment phase of selection (round 1), phage representing 1000-fold coverage of the library was incubated with naked, washed MyOne™ T1 beads and heat-denatured, bead-conjugated HLA monomer overnight at 4° C. on a rotator. This step was necessary to remove any phage recognizing either streptavidin or denatured monomer, present to a small extent in every preparation of biotinylated monomer. After negative selection, beads were isolated with a DynaMag-2 magnet (Life Technologies) and the supernatant containing unbound phage was transferred for positive selection against 1 μg of the mutant peptide-HLA monomer conjugated to MyOne™ T1 streptavidin magnetic beads. Prior to elution, beads were washed 10 times with 1 ml, 1×TBS containing 0.5% Tween®-20 using a magnet. Phage was eluted by resuspending the beads in 1 mL of 0.2 M glycine, pH 2.2. After a 10-minute incubation, the solution was neutralized by the addition of 150 μL of 1 M Tris, pH 9.0. Neutralized phage was used to infect 10 ml cultures of mid-log-phage SS320s, with the addition of M13K07 helper phage (MOI of 4) and 2% glucose. After shaking for 1 hour at 37° C., bacteria was resuspended in 2×YT medium with carbenicillin (100 μg/mL), kanamycin (50 μg/mL), and 50 uM of IPTG and grown overnight at 30° C. for phage production. Phage was precipitated the next morning with PEG/NaCl as previously described.

### Final Selection Phase.

Three to five rounds of final selection were performed with phage resulting from the enrichment phase. For each round of final selection, the first negative selection was performed using 10-0.1% of the precipitated phage against HLA-allele matched cells lacking the mutated protein of interest. The unbound phage was then negatively selected against native wt peptide-HLA monomer and unrelated HLA-allele matched monomer. After negative selection, beads were isolated with a Dynamag 2 magnet (Life Technologies) and the supernatant containing unbound phage was transferred for positive selection with 250 ng to 1 μg of mutant peptide-HLA monomer, as described for the enrichment phase above.

### ELISA.

Streptavidin-coated, 96-well plates (R&D Systems, Minneapolis, MN) were coated with 50 ng (in 50 uL) of biotinylated mutant or wt peptide-HLA monomers in blocking buffer (PBS with 0.5% BSA, 2 mM EDTA, and 0.1% sodium azide) at 4° C. overnight. Plates were briefly washed with 1×TBST (TBS+0.05% Trition-X 100). Phage was serially diluted to the specified concentrations in blocking buffer and 50 uL was added to each well. Phage were incubated for 2 hrs at RT, followed by washing (6 washes with 1×TBS-0.05% Tween®-20 (TBST) using an ELISA plate washer (BioTek, Winooski, VT). The bound phage were incubated with 50 μL of rabbit anti-M13 antibody (Pierce, Rockford, IL) diluted 1:3000 in 1×TBST for 1 hr at room temperature, followed by washing an additional 6× times and incubation with 50 μL of anti-Rabbit HRP (Thermo Fisher) diluted 1:10,000 in 1×TBST for 1 hour at room temperature. After a final 6 washes with 1×TBST, 50 L of TMB substrate (BioLegend, San Diego, CA) was added to the well and the reaction was quenched with IN sulfuric acid. Absorbance at 450 nm was measured with a Synergy H1 Multi-Mode Reader (BioTek, Winooski, VT).

Monoclonal phage ELISA was performed by selecting individual colonies of SS320 cells transformed with a limiting dilution of phage obtained from the final selection. Individual colonies were inoculated into 200 μl of 2×YT medium containing 100 μg/mL carbenicillin and 2% glucose and grown for three hours at 37° C. The cells were then infected with 1.6×10⁷ M13K07 helper phage (Antibody Design Labs, San Diego, CA) and incubated for at 37° C. with shaking. The cells were pelleted, resuspended in 300 μL of 2×YT medium containing carbenicillin (100 μg/mL), kanamycin (50 μg/mL), and 50 μM IPTG, and grown overnight at 30° C. Cells were pelleted and the phage-laden supernatant was used for ELISA as described above.

### Peptide Pulsing and Flow Cytometry.

For peptide pulsing, HLA-matched cells were washed once with PBS and once with serum-free RPMI-1640 before incubation at 10⁶ cells per mL in serum-free RPMI-1640 containing 50 μg/mL peptide and 10 μg/mL human beta-2 microglobulin (ProSpec, East Brunswick, NJ) overnight at 37° C. The pulsed cells were pelleted, washed once in cold stain buffer (PBS containing 0.5% BSA, 2 mM EDTA, and 0.1% sodium azide), and resuspended in 100 μL of stain buffer. Phage staining was performed on ice with 10 uL (approximately 1×10⁹) phage for 1 hour in 100 uL total volume, followed by one 4 mL wash in cold stain buffer. Cells were then stained with 1 uL of rabbit anti-M13 antibody (Pierce, Rockford, IL) in 100 uL total volume on ice for 1 hour and washed once with 4 mL of cold stain buffer. Cells were stained with anti-rabbit-PE (Biolegend) on ice for 1 hour in 100 uL total volume, followed by incubation with LIVE/DEAD Fixable Near-IR Dead Cell Stain (Thermo Fisher) for 10 min at room temperature per manufacturer's instructions. Cells were washed once in 4 mL of stain buffer followed by resuspension in 300 uL of stain buffer before analysis. Stained cells were analyzed using an LSRII flow cytometer (Becton Dickinson, Mansfield, MA).

### CAR Construction and Generation.

A third-generation Chimeric Antigen Receptor (CAR) construct, containing the MANAbody scFv, a CD28 transmembrane domain, and 4-1BB and CD3ζ intracellular domains, was synthesized (GeneArt®) and cloned into the mammalian expression vector pCI (PROMEGA®). mRNA was synthesized with the T7 mScript™ Standard mRNA Production System Kit (CellScript™) per manufacturer's instructions. CAR mRNA was electroporated into primary CD3+ T cells with the BTX ECM 2001 Electro Cell Manipulator (Harvard Apparatus) to generate CAR-T cells.

### CAR-T Activation Co-Culture Assay.

COS-7 cells were transfected with various combinations of pcDNA3.1 (Life Technologies) plasmids encoding HLA-A3, HLA-B7, IDH2(WT), IDH2(R140Q), KRAS(WT), and KRAS(G12V) with Lipofectamine 3000 (Life Technologies) per manufacturer's instructions in 96-well plate format. 100,000 electroporated CAR-T cells were overlaid over the transfected COS-7 cells, and the co-culture was allowed to incubate for 4 hours at 37° C. under 5% CO₂. Following co-culture, conditioned media was collected and assayed for secreted IFNγ by ELISA (Quantikine®, R&D Systems).

### Bispecific Antibody Production.

gBLOCKs encoding bispecific antibodies were ordered from IDT (Skokie, Illinois). gBLOCKs were topo-cloned into the pcDNA3.4 plasmid (Thermo Fisher) following the manufacturer's protocol. 293FT cells (Thermo Fisher) were transfected with the bispecific antibody pcDNA3.4 plasmids using Lipofectamine 3000 (Life Technologies) per manufacturer's instructions in a T75 flask. Following a 5-7 day incubation, media was harvest and centrifuged at 3,000 g for 10 min at 4 C. Bispecific antibody protein was purified using a Clontech Capturem™ His-Tagged Purification Miniprep Kit (Takara, Mountain View, CA) per manufacturer's instructions. Bispecific antibody protein was desalted into PBS using Zeba spin 7 k MWCO desalting columns per manufacturer's instructions. Bispecific antibody concentration was quantified using Mini-PROTEAN® TGX Stain-Free™ Precast Gels (Biorad, Hercules, California) using a standard curve of protein of known concentration. Stain-free gels were imaged using the ChemiDoc XRS+ Imager (Biorad).

### Bispecific Antibody Co-Culture Assay.

COS-7 cells were transfected with various combinations of pcDNA3.1 (Life Technologies) plasmids encoding HLA-A3, HLA-B7, IDH2(WT), IDH2(R140Q), KRAS(WT), and KRAS(G12V) with Lipofectamine 3000 (Life Technologies) per manufacturer's instructions in a T75 flask. 50,000 T cells were combined with transfected 30,000 COS-7 cells or 10,000 NCI-H441 cells and the specified concentration of bispecific antibody in a 96-well plate, and the co-culture was allowed to incubate for 24 hours at 37° C. under 5% CO₂. Following co-culture, the 96-well plate was snap frozen and conditioned media lysate was collected and assayed for secreted IFNγ by ELISA (Quantikine®, R&D Systems). Alternatively, following coculture, target cell viability was measured using CellTiter-Glo® (PROMEGA®).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A molecule comprising an antigen-binding domain that can bind to a peptide-HLA-beta-2 microglobulin complex, wherein said peptide comprises a modified peptide, wherein said HLA is a class I HLA, and wherein said antigen-binding domain does not bind to a complex that includes a wild-type version of the modified peptide, wherein said modified peptide is derived from a modified KRAS polypeptide, wherein said modified peptide comprises an amino acid sequence selected from the group consisting SEQ ID NO: 18, SEQ ID NO:20, SEQ ID NO:21, SEQ ID NO:22 and SEQ ID NO:24.

2. The molecule of claim 1, wherein said modified peptide comprises from 7 amino acids to 15 amino acids.

3. The molecule of claim 2, wherein said modified peptide comprises 10 amino acids.

4-10. (canceled)

11. The molecule of claim 1, wherein said modified peptide comprises SEQ ID NO:18, wherein said class I HLA is an HLA-A2, and wherein said antigen binding fragment comprises an amino acid sequence selected from the group consisting of SEQ ID NO:338, SEQ ID NO:339, and SEQ ID NO:340.

12. The molecule of claim 1, wherein said modified peptide comprises SEQ ID NO:20, wherein said class I HLA is an HLA-A3, and wherein said antigen binding fragment comprises an amino acid sequence selected from the group consisting of SEQ ID NO:341, SEQ ID NO:342, and SEQ ID NO:343.

13. The molecule of claim 1, wherein said modified peptide comprises SEQ ID NO:21, wherein said class I HLA is an HLA-A3, and wherein said antigen binding fragment comprises an amino acid sequence selected from the group consisting of SEQ ID NO:342, SEQ ID NO:343, SEQ ID NO:349, SEQ ID NO:350, SEQ ID NO:351, SEQ ID NO:352, SEQ ID NO:353, SEQ ID NO:354, SEQ ID NO:355, SEQ ID NO:356, and SEQ ID NO:357.

14. The molecule of claim 1, wherein said modified peptide comprises SEQ ID NO:22, wherein said class I HLA is an HLA-A3, and wherein said antigen binding fragment comprises an amino acid sequence selected from the group consisting of SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:369, SEQ ID NO:370, SEQ ID NO:371, SEQ ID NO:372, SEQ ID NO:373, and SEQ ID NO:374.

15. The molecule of claim 1, wherein said modified peptide comprises SEQ ID NO:24, wherein said class I HLA is an HLA-A11, and wherein said antigen binding fragment comprises an amino acid sequence selected from the group consisting of SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, and SEQ ID NO:368.

16-20. (canceled)

21. The molecule of claim 1, wherein said molecule is selected from the group consisting of an antibody, an antibody fragment, a single chain variable fragment (scFv), a chimeric antigen receptor (CAR), a T cell receptor (TCR), a TCR mimic, a tandem scFv, a bispecific T cell engager, a diabody, a single-chain diabody, an scFv-Fc, a bispecific antibody, and a dual-affinity re-targeting antibody (DART).

22. The molecule of claim 1, wherein said molecule is a single-chain diabody.

23. The molecule of claim 1, wherein said molecule further comprises an antigen-binding domain that can bind to an effector cell receptor selected from the group consisting of CD3, CD28, CD4, CD8, CD16a, NKG2D, PD-1, CTLA-4, 4-1BB, OX40, ICOS, and CD27.

24. The molecule of claim 23, wherein said antigen-binding domain that can bind to an effector cell can bind to CD3, wherein said antigen-binding domain comprises an amino acid sequence selected from the group consisting of SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, and SEQ ID NO:417.

25. The molecule of claim 23, wherein said antigen-binding domain that can bind to an effector cell can bind to CD16a.

26. The molecule of claim 23, wherein said antigen-binding domain that can bind to an effector cell can bind to NKG2D.

27-35. (canceled)


SEQUENCE LISTING (420 sequences)

SEQ ID NO: 1 - protein, 10 aa, synthetic construct
SPNGTIQNIL

SEQ ID NO: 2 - protein, 10 aa, synthetic construct
SPNGTIRNIL

SEQ ID NO: 3 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYSPPTFGQGTKVEIKRTGGGSGGGGSGGGASEVQLVESGGGLVQPGGSLRLSCAASGFNISDTYIHWVRQAPGKGLEWVASISPRTGYNRYADSVKGRFTISADTSKNTAYLQMNSLRAEDTAVYYCSRAYYSYAYAMDVWGQGTLVTVSS

SEQ ID NO: 4 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGKAYWPATFGQGTKVEIKRTGGGSGGGGSGGGASEVQLVESGGGLVQPGGSLRLSCAASGFNVGHYRMHWVRQAPGKGLEWVAMVSPNGYYTYYADSVKGRFTISADTSKNTAYLQMNSLRAEDTAVYYCSRGYSSYAFDYWGQGTLVTVSS

SEQ ID NO: 5 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQVYSSPFTFGQGTKVEIKRTGGGSGGGGSGGGASEVQLVESGGGLVQPGGSLRLSCAASGFNVKYYMMHWVRQAPGKGLEWVAAISPGYDYTYYADSVKGRFTISADTSKNTAYLQMNSLRAEDTAVYYCSRSYWRYSVDVWGQGTLVTVSS

SEQ ID NO: 6 - protein, 245 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSLYSPMTFGQGTKVEIKRTGGGSGGGGSGGGASEVQLVESGGGLVQPGGSLRLSCAASGFNSFLSIHWVRQAPGKGLEWVAHIFPSSDYTSYADSVKGRFTISADTSKNTAYLQMNSLRAEDTAVYYCSRGKHSSDSNYYMDYWGQGTLVTVSS

SEQ ID NO: 7 - skipped

SEQ ID NO: 8 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYMPFTFGQGTKVEIKRTGGGSGGGGSGGGASEVQLVESGGGLVQPGGSLRLSCAASGFNIFRGYMHWVRQAPGKGLEWVAMISPHSDYTSYADSVKGRFTISADTSKNTAYLQMNSLRAEDTAVYYCSRSYGWAAFDYWGQGTLVTVSS

SEQ ID NO: 9 - protein, 10 aa, synthetic construct
RPIPIKYKAM

SEQ ID NO: 10 - protein, 10 aa, synthetic construct
KPITIGRHAH

SEQ ID NO: 11 - protein, 9 aa, synthetic construct
AVGVGKSAL

SEQ ID NO: 12 - protein, 9 aa, synthetic construct
AGGVGKSAL

SEQ ID NO: 13 - protein, 9 aa, synthetic construct
IMQLMPFGC

SEQ ID NO: 14 - protein, 9 aa, synthetic construct
ITQLMPFGC

SEQ ID NO: 15 - protein, 10 aa, synthetic construct
GMNQRPILTI

SEQ ID NO: 16 - protein, 10 aa, synthetic construct
GMNWRPILTI

SEQ ID NO: 17 - protein, 10 aa, synthetic construct
GMNRRPILTI

SEQ ID NO: 18 - protein, 9 aa, synthetic construct
LVVVGAVGV

SEQ ID NO: 19 - protein, 9 aa, synthetic construct
LVVVGAGGV

SEQ ID NO: 20 - protein, 10 aa, synthetic construct
VVVGACGVGK

SEQ ID NO: 21 - protein, 10 aa, synthetic construct
VVVGADGVGK

SEQ ID NO: 22 - protein, 9 aa, synthetic construct
VVGAVGVGK

SEQ ID NO: 23 - protein, 10 aa, synthetic construct
VVVGAGGVGK

SEQ ID NO: 24 - protein, 9 aa, synthetic construct
VVGADGVGK

SEQ ID NO: 25 - protein, 9 aa, synthetic construct
VVGAGGVGK

SEQ ID NO: 26 - protein, 9 aa, synthetic construct
TTAPFLSGK

SEQ ID NO: 27 - protein, 9 aa, synthetic construct
TTAPSLSGK

SEQ ID NO: 28 - protein, 10 aa, synthetic construct
ILDTAGHEEY

SEQ ID NO: 29 - protein, 10 aa, synthetic construct
ILDTAGQEEY

SEQ ID NO: 30 - protein, 10 aa, synthetic construct
ILDTAGKEEY

SEQ ID NO: 31 - protein, 10 aa, synthetic construct
ILDTAGLEEY

SEQ ID NO: 32 - protein, 10 aa, synthetic construct
ILDTAGREEY

SEQ ID NO: 33 - protein, 6 aa, synthetic construct
QDVNTA

SEQ ID NO: 34 - protein, 9 aa, synthetic construct
QQYDYAPIT

SEQ ID NO: 35 - protein, 11 aa, synthetic construct
QQSPYYYLPIT

SEQ ID NO: 36 - protein, 9 aa, synthetic construct
QQYYYSPVT

SEQ ID NO: 37 - protein, 9 aa, synthetic construct
QQHYGNPFT

SEQ ID NO: 38 - protein, 9 aa, synthetic construct
QQSYYSPPT

SEQ ID NO: 39 - protein, 9 aa, synthetic construct
QQYYSYPPT

SEQ ID NO: 40 - protein, 9 aa, synthetic construct
QQYYYYPPT

SEQ ID NO: 41 - protein, 8 aa, synthetic construct
GFNISWYQ

SEQ ID NO: 42 - protein, 8 aa, synthetic construct
GFNVSWSY

SEQ ID NO: 43 - protein, 8 aa, synthetic construct
GFNISWNQ

SEQ ID NO: 44 - protein, 8 aa, synthetic construct
GFNVGYYG

SEQ ID NO: 45 - protein, 8 aa, synthetic construct
GFNITSSY

SEQ ID NO: 46 - protein, 8 aa, synthetic construct
GFNINSSY

SEQ ID NO: 47 - protein, 8 aa, synthetic construct
GFNISTSY

SEQ ID NO: 48 - protein, 8 aa, synthetic construct
VTPYSGYT

SEQ ID NO: 49 - protein, 8 aa, synthetic construct
IYGDSGYT

SEQ ID NO: 50 - protein, 8 aa, synthetic construct
VSPYSGYT

SEQ ID NO: 51 - protein, 8 aa, synthetic construct
VSGMEGYT

SEQ ID NO: 52 - protein, 8 aa, synthetic construct
ISPADGYN

SEQ ID NO: 53 - protein, 8 aa, synthetic construct
ISPTDGYY

SEQ ID NO: 54 - protein, 8 aa, synthetic construct
IDPNDGYS

SEQ ID NO: 55 - protein, 10 aa, synthetic construct
SRSYTDGFDY

SEQ ID NO: 56 - protein, 14 aa, synthetic construct
SRGQWEASYYAMDY

SEQ ID NO: 57 - protein, 10 aa, synthetic construct
SRSDYYAMDY

SEQ ID NO: 58 - protein, 11 aa, synthetic construct
SRDIYGYAMDV

SEQ ID NO: 59 - protein, 13 aa, synthetic construct
SRTDSTAYTAMDV

SEQ ID NO: 60 - protein, 13 aa, synthetic construct
SRTSDTSYAAMDV

SEQ ID NO: 61 - protein, 13 aa, synthetic construct
SRTNNTAADAMDV

SEQ ID NO: 62 - protein, 9 aa, synthetic construct
QQYSYSPPT

SEQ ID NO: 63 - protein, 10 aa, synthetic construct
QQGKAYWPAT

SEQ ID NO: 64 - protein, 9 aa, synthetic construct
QQVYSSPFT

SEQ ID NO: 65 - protein, 10 aa, synthetic construct
QQYSLYSPMT

SEQ ID NO: 66 - protein, 9 aa, synthetic construct
QQSYYMPFT

SEQ ID NO: 67 - protein, 8 aa, synthetic construct
GFNISDTY

SEQ ID NO: 68 - protein, 8 aa, synthetic construct
GFNVGHYR

SEQ ID NO: 69 - protein, 8 aa, synthetic construct
GFNVKYYM

SEQ ID NO: 70 - protein, 7 aa, synthetic construct
GFNSFLS

SEQ ID NO: 71 - protein, 8 aa, synthetic construct
GFNIFRGY

SEQ ID NO: 72 - protein, 8 aa, synthetic construct
ISPRTGYN

SEQ ID NO: 73 - protein, 8 aa, synthetic construct
VSPNGYYT

SEQ ID NO: 74 - protein, 8 aa, synthetic construct
ISPGYDYT

SEQ ID NO: 75 - protein, 8 aa, synthetic construct
IFPSSDYT

SEQ ID NO: 76 - protein, 8 aa, synthetic construct
ISPHSDYT

SEQ ID NO: 77 - protein, 13 aa, synthetic construct
SRAYYSYAYAMDV

SEQ ID NO: 78 - protein, 11 aa, synthetic construct
SRGYSSYAFDY

SEQ ID NO: 79 - protein, 11 aa, synthetic construct
SRSYWRYSVDV

SEQ ID NO: 80 - protein, 15 aa, synthetic construct
SRGKHSSDSNYYMDY

SEQ ID NO: 81 - protein, 11 aa, synthetic construct
SRSYGWAAFDY

SEQ ID NO: 82 - protein, 9 aa, synthetic construct
QQSGYAPIT

SEQ ID NO: 83 - protein, 9 aa, synthetic construct
QQYSYAPIT

SEQ ID NO: 84 - protein, 9 aa, synthetic construct
QQSLYGPFT

SEQ ID NO: 85 - protein, 9 aa, synthetic construct
QQYSYSPIT

SEQ ID NO: 86 - protein, 9 aa, synthetic construct
QQSGYQPDT

SEQ ID NO: 87 - protein, 9 aa, synthetic construct
QQYLYQPWT

SEQ ID NO: 88 - protein, 9 aa, synthetic construct
QQGLYYPWT

SEQ ID NO: 89 - protein, 8 aa, synthetic construct
GFNISYYS

SEQ ID NO: 90 - protein, 8 aa, synthetic construct
GFNIGYYT

SEQ ID NO: 91 - protein, 8 aa, synthetic construct
GFNIAYEY

SEQ ID NO: 92 - protein, 8 aa, synthetic construct
GFNLFGYG

SEQ ID NO: 93 - protein, 8 aa, synthetic construct
GFNISWYA

SEQ ID NO: 94 - protein, 8 aa, synthetic construct
GFNIDYYG

SEQ ID NO: 95 - protein, 8 aa, synthetic construct
GFNVSYSS

SEQ ID NO: 96 - protein, 8 aa, synthetic construct
VDPDSDYT

SEQ ID NO: 97 - protein, 8 aa, synthetic construct
VSPWSYST

SEQ ID NO: 98 - protein, 8 aa, synthetic construct
IGPDSGYT

SEQ ID NO: 99 - protein, 8 aa, synthetic construct
IGPYYYYT

SEQ ID NO: 100 - protein, 8 aa, synthetic construct
IWPDSDWT

SEQ ID NO: 101 - protein, 8 aa, synthetic construct
LYGGSDST

SEQ ID NO: 102 - protein, 8 aa, synthetic construct
IWPDSGQT

SEQ ID NO: 103 - protein, 12 aa, synthetic construct
SRSWIHMFSMDY

SEQ ID NO: 104 - protein, 11 aa, synthetic construct
SRDHWDEAFDV

SEQ ID NO: 105 - protein, 13 aa, synthetic construct
SRVWYYSTYGMDY

SEQ ID NO: 106 - protein, 11 aa, synthetic construct
SRENYDMAMDY

SEQ ID NO: 107 - protein, 11 aa, synthetic construct
SRYYYSSAFDV

SEQ ID NO: 108 - protein, 10 aa, synthetic construct
SRQYSAYFDY

SEQ ID NO: 109 - protein, 11 aa, synthetic construct
SRSSYFDAMDY

SEQ ID NO: 110 - protein, 9 aa, synthetic construct
QQWYSSPVT

SEQ ID NO: 111 - protein, 8 aa, synthetic construct
GFNINWAN

SEQ ID NO: 112 - protein, 8 aa, synthetic construct
GFNINWAN

SEQ ID NO: 113 - protein, 8 aa, synthetic construct
GFNIYLHD

SEQ ID NO: 114 - protein, 8 aa, synthetic construct
GFNIYWSH

SEQ ID NO: 115 - protein, 8 aa, synthetic construct
ISPPYDYT

SEQ ID NO: 116 - protein, 8 aa, synthetic construct
IIPAIDYT

SEQ ID NO: 117 - protein, 8 aa, synthetic construct
ISSFEGYT

SEQ ID NO: 118 - protein, 10 aa, synthetic construct
SRSYSYYFDY

SEQ ID NO: 119 - protein, 10 aa, synthetic construct
SRRDGYYFDY

SEQ ID NO: 120 - protein, 10 aa, synthetic construct
SRSYSYYMDY

SEQ ID NO: 121 - protein, 11 aa, synthetic construct
QQSYGSGSPWT

SEQ ID NO: 122 - protein, 9 aa, synthetic construct
QQTYYSPWT

SEQ ID NO: 123 - protein, 9 aa, synthetic construct
QQYYYPPIT

SEQ ID NO: 124 - protein, 8 aa, synthetic construct
GFNIVGGG

SEQ ID NO: 125 - protein, 8 aa, synthetic construct
GFNIRSYA

SEQ ID NO: 126 - protein, 8 aa, synthetic construct
GFNVSHTG

SEQ ID NO: 127 - protein, 8 aa, synthetic construct
IYPQGDYT

SEQ ID NO: 128 - protein, 8 aa, synthetic construct
VGPGKGYT

SEQ ID NO: 129 - protein, 11 aa, synthetic construct
SRDSSYLAFDY

SEQ ID NO: 130 - protein, 13 aa, synthetic construct
SRNFQSTSHAFDY

SEQ ID NO: 131 - protein, 10 aa, synthetic construct
SRKTYYAFDY

SEQ ID NO: 132 - protein, 11 aa, synthetic construct
QQSYYYFRPIT

SEQ ID NO: 133 - protein, 10 aa, synthetic construct
QQASYYYPLT

SEQ ID NO: 134 - protein, 10 aa, synthetic construct
QQKSEYSPWT

SEQ ID NO: 135 - protein, 9 aa, synthetic construct
QQSGYIPFT

SEQ ID NO: 136 - protein, 10 aa, synthetic construct
QQGAYYRPFT

SEQ ID NO: 137 - protein, 8 aa, synthetic construct
GFNLSYSD

SEQ ID NO: 138 - protein, 8 aa, synthetic construct
GFNISASG

SEQ ID NO: 139 - protein, 8 aa, synthetic construct
GFNIYRYG

SEQ ID NO: 140 - protein, 8 aa, synthetic construct
GFNIYGTM

SEQ ID NO: 141 - protein, 8 aa, synthetic construct
GFNISYSY

SEQ ID NO: 142 - protein, 8 aa, synthetic construct
VMPDSGHT

SEQ ID NO: 143 - protein, 8 aa, synthetic construct
IHPLKPYT

SEQ ID NO: 144 - protein, 8 aa, synthetic construct
LYPYGYST

SEQ ID NO: 145 - protein, 8 aa, synthetic construct
FKPDSYNT

SEQ ID NO: 146 - protein, 8 aa, synthetic construct
LLPYDGNT

SEQ ID NO: 147 - protein, 13 aa, synthetic construct
SRATNIPVYAFDY

SEQ ID NO: 148 - protein, 13 aa, synthetic construct
SRYSSMYYYYFDY

SEQ ID NO: 149 - protein, 11 aa, synthetic construct
SRSYAYGYFAY

SEQ ID NO: 150 - protein, 13 aa, synthetic construct
SRGEVYHYYAFDY

SEQ ID NO: 151 - protein, 10 aa, synthetic construct
SRAAYSSMDV

SEQ ID NO: 152 - protein, 9 aa, synthetic construct
QQYMYSPVT

SEQ ID NO: 153 - protein, 9 aa, synthetic construct
QQSSSSPIT

SEQ ID NO: 154 - protein, 9 aa, synthetic construct
QQSSASPLT

SEQ ID NO: 155 - protein, 9 aa, synthetic construct
QQYAYSPLT

SEQ ID NO: 156 - protein, 8 aa, synthetic construct
GFNVSAYW

SEQ ID NO: 157 - protein, 8 aa, synthetic construct
GFNISGYG

SEQ ID NO: 158 - protein, 8 aa, synthetic construct
GFNVSSVG

SEQ ID NO: 159 - protein, 8 aa, synthetic construct
GFNVSSYG

SEQ ID NO: 160 - protein, 8 aa, synthetic construct
IYGGSGYT

SEQ ID NO: 161 - protein, 8 aa, synthetic construct
LYGGSDYT

SEQ ID NO: 162 - protein, 8 aa, synthetic construct
IYGTSDYT

SEQ ID NO: 163 - protein, 8 aa, synthetic construct
IAPRRDYT

SEQ ID NO: 164 - protein, 12 aa, synthetic construct
SRTHSYWSAFDY

SEQ ID NO: 165 - protein, 10 aa, synthetic construct
SRTVRYAFDY

SEQ ID NO: 166 - protein, 10 aa, synthetic construct
SRSSRYSMDY

SEQ ID NO: 167 - protein, 10 aa, synthetic construct
SRKSSYYFDY

SEQ ID NO: 168 - protein, 9 aa, synthetic construct
QQYSYYPIT

SEQ ID NO: 169 - protein, 9 aa, synthetic construct
QQYSYTPVT

SEQ ID NO: 170 - protein, 9 aa, synthetic construct
QQYSYEPVT

SEQ ID NO: 171 - protein, 10 aa, synthetic construct
QQYAYYSPVT

SEQ ID NO: 172 - protein, 9 aa, synthetic construct
QQYEYYPMT

SEQ ID NO: 173 - protein, 8 aa, synthetic construct
GFNFSYGY

SEQ ID NO: 174 - protein, 8 aa, synthetic construct
GFNVWGPG

SEQ ID NO: 175 - protein, 8 aa, synthetic construct
GFNVSGSQ

SEQ ID NO: 176 - protein, 8 aa, synthetic construct
GFNIYGQM

SEQ ID NO: 177 - protein, 8 aa, synthetic construct
GFNVMYST

SEQ ID NO: 178 - protein, 8 aa, synthetic construct
ISGYTGNT

SEQ ID NO: 179 - protein, 8 aa, synthetic construct
IHPFSGNT

SEQ ID NO: 180 - protein, 8 aa, synthetic construct
IPGWSGYT

SEQ ID NO: 181 - protein, 8 aa, synthetic construct
LSPFSGNT

SEQ ID NO: 182 - protein, 8 aa, synthetic construct
IYSWSDYT

SEQ ID NO: 183 - protein, 16 aa, synthetic construct
SRAASLSSSYYSAFDV

SEQ ID NO: 184 - protein, 11 aa, synthetic construct
SRGYSYSAMDY

SEQ ID NO: 185 - protein, 11 aa, synthetic construct
SRGYSYFAMDY

SEQ ID NO: 186 - protein, 14 aa, synthetic construct
SRNISYEQSSAFDY

SEQ ID NO: 187 - protein, 11 aa, synthetic construct
SRGYAHNSFDY

SEQ ID NO: 188 - protein, 9 aa, synthetic construct
QQYSFYPFT

SEQ ID NO: 189 - protein, 11 aa, synthetic construct
QQYSAYYQPIT

SEQ ID NO: 190 - protein, 9 aa, synthetic construct
QQYEYVPHT

SEQ ID NO: 191 - protein, 9 aa, synthetic construct
QQYSYMPIT

SEQ ID NO: 192 - protein, 9 aa, synthetic construct
QQYAYYPVT

SEQ ID NO: 193 - protein, 9 aa, synthetic construct
QQYDYRPVT

SEQ ID NO: 194 - protein, 9 aa, synthetic construct
QQYDFTPMT

SEQ ID NO: 195 - protein, 10 aa, synthetic construct
QQYSSSSPVT

SEQ ID NO: 196 - protein, 7 aa, synthetic construct
GFNFGSY

SEQ ID NO: 197 - protein, 8 aa, synthetic construct
GFNISDSY

SEQ ID NO: 198 - protein, 8 aa, synthetic construct
GFNIFSDQ

SEQ ID NO: 199 - protein, 8 aa, synthetic construct
GFNLSYSY

SEQ ID NO: 200 - protein, 8 aa, synthetic construct
GFNISYGY

SEQ ID NO: 201 - protein, 8 aa, synthetic construct
GFNISYQH

SEQ ID NO: 202 - protein, 8 aa, synthetic construct
GFNLSGYY

SEQ ID NO: 203 - protein, 8 aa, synthetic construct
GFNVSGQY

SEQ ID NO: 204 - protein, 8 aa, synthetic construct
GFNVSTSG

SEQ ID NO: 205 - protein, 8 aa, synthetic construct
GFNISYAK

SEQ ID NO: 206 - protein, 8 aa, synthetic construct
GFNFSSYV

SEQ ID NO: 207 - protein, 8 aa, synthetic construct
ISGYSGNT

SEQ ID NO: 208 - protein, 8 aa, synthetic construct
FSPYSSNT

SEQ ID NO: 209 - protein, 9 aa, synthetic construct
FMPYDSYYT

SEQ ID NO: 210 - protein, 8 aa, synthetic construct
ISGFSGNT

SEQ ID NO: 211 - protein, 8 aa, synthetic construct
FHYGSGNT

SEQ ID NO: 212 - protein, 8 aa, synthetic construct
FMPYQGST

SEQ ID NO: 213 - protein, 8 aa, synthetic construct
FSPYSGYT

SEQ ID NO: 214 - protein, 8 aa, synthetic construct
ISPVSGNT

SEQ ID NO: 215 - protein, 8 aa, synthetic construct
IYGAYSGT

SEQ ID NO: 216 - protein, 8 aa, synthetic construct
LTYWGGYT

SEQ ID NO: 217 - protein, 8 aa, synthetic construct
VYPDSGGT

SEQ ID NO: 218 - protein, 13 aa, synthetic construct
SRSNQSAYSYMDY

SEQ ID NO: 219 - protein, 13 aa, synthetic construct
SRSQFTFYQYFDY

SEQ ID NO: 220 - protein, 11 aa, synthetic construct
SRMSVRNAFDY

SEQ ID NO: 221 - protein, 12 aa, synthetic construct
SRSDSYYTAMDY

SEQ ID NO: 222 - protein, 10 aa, synthetic construct
SRSNYYYLDY

SEQ ID NO: 223 - protein, 14 aa, synthetic construct
SRANIYSSHSFFDY

SEQ ID NO: 224 - protein, 13 aa, synthetic construct
SRTHSSIYHSFDY

SEQ ID NO: 225 - protein, 14 aa, synthetic construct
SRPMKTSYYGAFDY

SEQ ID NO: 226 - protein, 14 aa, synthetic construct
SRSQSYTYWSAMDY

SEQ ID NO: 227 - protein, 11 aa, synthetic construct
SRGEYGTYMDY

SEQ ID NO: 228 - protein, 11 aa, synthetic construct
SRTSSYYAFDY

SEQ ID NO: 229 - protein, 8 aa, synthetic construct
QQSSYTPI

SEQ ID NO: 230 - protein, 9 aa, synthetic construct
QQYAYYPIT

SEQ ID NO: 231 - protein, 9 aa, synthetic construct
QQYEYYPIT

SEQ ID NO: 232 - protein, 9 aa, synthetic construct
QQYTYYPIT

SEQ ID NO: 233 - protein, 9 aa, synthetic construct
QQSSVEPWT

SEQ ID NO: 234 - protein, 8 aa, synthetic construct
GFNISQGG

SEQ ID NO: 235 - protein, 8 aa, synthetic construct
GFNISSTG

SEQ ID NO: 236 - protein, 8 aa, synthetic construct
GFNFFSTV

SEQ ID NO: 237 - protein, 8 aa, synthetic construct
GFNLHGYL

SEQ ID NO: 238 - protein, 8 aa, synthetic construct
GFNLSTHV

SEQ ID NO: 239 - protein, 8 aa, synthetic construct
GFNVSYYS

SEQ ID NO: 240 - protein, 8 aa, synthetic construct
VYPGGGQT

SEQ ID NO: 241 - protein, 8 aa, synthetic construct
LLGGSGNT

SEQ ID NO: 242 - protein, 8 aa, synthetic construct
IYPWSGST

SEQ ID NO: 243 - protein, 8 aa, synthetic construct
IYPPNGYT

SEQ ID NO: 244 - protein, 8 aa, synthetic construct
FYPYVGYT

SEQ ID NO: 245 - protein, 8 aa, synthetic construct
IYPWNDYT

SEQ ID NO: 246 - protein, 11 aa, synthetic construct
SRGYDYSAFDY

SEQ ID NO: 247 - protein, 11 aa, synthetic construct
SRGLQYSAMDY

SEQ ID NO: 248 - protein, 12 aa, synthetic construct
SRSRSSNYYFDV

SEQ ID NO: 249 - protein, 11 aa, synthetic construct
SRGVDYAYLDY

SEQ ID NO: 250 - protein, 11 aa, synthetic construct
SRGYRYQYMDV

SEQ ID NO: 251 - protein, 10 aa, synthetic construct
SRGSYYSFDY

SEQ ID NO: 252 - protein, 9 aa, synthetic construct
QQIYTSPIT

SEQ ID NO: 253 - protein, 9 aa, synthetic construct
QQRAYFPIT

SEQ ID NO: 254 - protein, 10 aa, synthetic construct
QQQYAYTPIT

SEQ ID NO: 255 - protein, 9 aa, synthetic construct
QQIHYKPLT

SEQ ID NO: 256 - protein, 8 aa, synthetic construct
GFNINNTY

SEQ ID NO: 257 - protein, 8 aa, synthetic construct
GFNFITTG

SEQ ID NO: 258 - protein, 8 aa, synthetic construct
GFNFSDYG

SEQ ID NO: 259 - protein, 8 aa, synthetic construct
GFNVWSYG

SEQ ID NO: 260 - protein, 8 aa, synthetic construct
GFNVAWYS

SEQ ID NO: 261 - protein, 8 aa, synthetic construct
IGPGSDYT

SEQ ID NO: 262 - protein, 8 aa, synthetic construct
LIPASGYT

SEQ ID NO: 263 - protein, 8 aa, synthetic construct
VTPDGSYT

SEQ ID NO: 264 - protein, 8 aa, synthetic construct
VYGGSSYT

SEQ ID NO: 265 - protein, 13 aa, synthetic construct
SRTYYSYYSAMDV

SEQ ID NO: 266 - protein, 11 aa, synthetic construct
SRYYYASALDY

SEQ ID NO: 267 - protein, 10 aa, synthetic construct
SRGWSYYMDY

SEQ ID NO: 268 - protein, 10 aa, synthetic construct
SRSYGWAMDY

SEQ ID NO: 269 - protein, 11 aa, synthetic construct
SRDFYSSGMDY

SEQ ID NO: 270 - protein, 9 aa, synthetic construct
QQEWRLPIT

SEQ ID NO: 271 - protein, 9 aa, synthetic construct
QQGTSTPFT

SEQ ID NO: 272 - protein, 9 aa, synthetic construct
QQSWRYPMT

SEQ ID NO: 273 - protein, 9 aa, synthetic construct
QQSYSYPVT

SEQ ID NO: 274 - protein, 10 aa, synthetic construct
QQGWLYSPFT

SEQ ID NO: 275 - protein, 8 aa, synthetic construct
GFNVYGNQ

SEQ ID NO: 276 - protein, 8 aa, synthetic construct
GFNISRYG

SEQ ID NO: 277 - protein, 8 aa, synthetic construct
GFNIYSSW

SEQ ID NO: 278 - protein, 8 aa, synthetic construct
IYPYSGST

SEQ ID NO: 279 - protein, 8 aa, synthetic construct
IYPDSGYT

SEQ ID NO: 280 - protein, 8 aa, synthetic construct
FYPSSSYT

SEQ ID NO: 281 - protein, 8 aa, synthetic construct
FQPYSGYT

SEQ ID NO: 282 - protein, 8 aa, synthetic construct
VYGGSGYT

SEQ ID NO: 283 - protein, 13 aa, synthetic construct
SRSAYVAYSYFDY

SEQ ID NO: 284 - protein, 11 aa, synthetic construct
SRAYLYYYLAY

SEQ ID NO: 285 - protein, 10 aa, synthetic construct
SRKYYEAMDY

SEQ ID NO: 286 - protein, 10 aa, synthetic construct
SREYTYYFDY

SEQ ID NO: 287 - protein, 11 aa, synthetic construct
SRAHSSYYVDY

SEQ ID NO: 288 - protein, 10 aa, synthetic construct
QQGYFYYPNT

SEQ ID NO: 289 - protein, 8 aa, synthetic construct
GFNIGYYG

SEQ ID NO: 290 - protein, 8 aa, synthetic construct
VYPGGGYT

SEQ ID NO: 291 - protein, 10 aa, synthetic construct
SRYYYYGFDY

SEQ ID NO: 292 - protein, 9 aa, synthetic construct
QQHYYSPVT

SEQ ID NO: 293 - protein, 9 aa, synthetic construct
QQHYYSPVT

SEQ ID NO: 294 - protein, 8 aa, synthetic construct
IYPDYDYT

SEQ ID NO: 295 - protein, 10 aa, synthetic construct
SRTYSVYMDY

SEQ ID NO: 296 - protein, 9 aa, synthetic construct
QQYAYAPFT

SEQ ID NO: 297 - protein, 8 aa, synthetic construct
GFNVSYSM

SEQ ID NO: 298 - protein, 8 aa, synthetic construct
VWGDGGVT

SEQ ID NO: 299 - protein, 10 aa, synthetic construct
SRGSYYAFDY

SEQ ID NO: 300 - protein, 9 aa, synthetic construct
QQAHMIPIT

SEQ ID NO: 301 - protein, 9 aa, synthetic construct
QQSVYDPIT

SEQ ID NO: 302 - protein, 9 aa, synthetic construct
QQSYTSPLT

SEQ ID NO: 303 - protein, 9 aa, synthetic construct
QQGQYSPFT

SEQ ID NO: 304 - skipped

SEQ ID NO: 305 - protein, 8 aa, synthetic construct
GFNFSFPG

SEQ ID NO: 306 - protein, 8 aa, synthetic construct
GFNISGSW

SEQ ID NO: 307 - protein, 8 aa, synthetic construct
GFNIYYGV

SEQ ID NO: 308 - protein, 8 aa, synthetic construct
GFNVSYEY

SEQ ID NO: 309 - skipped

SEQ ID NO: 310 - protein, 8 aa, synthetic construct
FVGYDGYT

SEQ ID NO: 311 - protein, 8 aa, synthetic construct
LYPDSDYT

SEQ ID NO: 312 - protein, 8 aa, synthetic construct
IYPDSSWT

SEQ ID NO: 313 - protein, 8 aa, synthetic construct
IYGGSDNT

SEQ ID NO: 314-315 - skipped (2 entries)

SEQ ID NO: 316 - protein, 11 aa, synthetic construct
SRDYYSFSMDY

SEQ ID NO: 317 - protein, 10 aa, synthetic construct
SRAHTYAFDY

SEQ ID NO: 318 - protein, 19 aa, synthetic construct
SRDQDFHYMNYYLSYALDY

SEQ ID NO: 319 - protein, 10 aa, synthetic construct
SRPLGSYFDY

SEQ ID NO: 320 - protein, 9 aa, synthetic construct
QQYWYLPTT

SEQ ID NO: 321 - protein, 8 aa, synthetic construct
GFNISWYD

SEQ ID NO: 322 - protein, 8 aa, synthetic construct
IEPSVGYT

SEQ ID NO: 323 - protein, 11 aa, synthetic construct
SRSYPYYYFDY

SEQ ID NO: 324 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDYAPITFGQGTKVEIKRTGGGSGGGGSGGGASEVQLVESGGGLVQPGGSLRLSCAASGFNISWYQMHWVRQAPGKGLEWVALVTPYSGYTYYADSVKGRFTISADTSKNTAYLQMNSLRAEDTAVYYCSRSYTDGFDYWGQGTLVTVSS

SEQ ID NO: 325 - protein, 246 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSPYYYLPITFGQGTKVEIKRTGGGSGGGGSGGGASEVQLVESGGGLVQPGGSLRLSCAASGFNVSWSYMHWVRQAPGKGLEWVANIYGDSGYTHYADSVKGRFTISADTSKNTAYLQMNSLRAEDTAVYYCSRGQWEASYYAMDYWGQGTLVTVSS

SEQ ID NO: 326 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYYSPVTFGQGTKVEIKRTGGGSGGGGSGGGASEVQLVESGGGLVQPGGSLRLSCAASGFNISWNQMHWVRQAPGKGLEWVALVSPYSGYTYYADSVKGRFTISADTSKNTAYLQMNSLRAEDTAVYYCSRSDYYAMDYWGQGTLVTVSS

SEQ ID NO: 327 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYGNPFTFGQGTKVEIKRTGGGSGGGGSGGGASEVQLVESGGGLVQPGGSLRLSCAASGFNVGYYGMHWVRQAPGKGLEWVAFVSGMEGYTSYADSVKGRFTISADTSKNTAYLQMNSLRAEDTAVYYCSRDIYGYAMDVWGQGTLVTVSS

SEQ ID NO: 328 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYSPPTFGQGTKVEIKRTGGGSGGGGSGGGASEVQLVESGGGLVQPGGSLRLSCAASGFNITSSYIHWVRQAPGKGLEWVAYISPADGYNRYADSVKGRFTISADTSKNTAYLQMNSLRAEDTAVYYCSRTDSTAYTAMDVWGQGTLVTVSS

SEQ ID NO: 329 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYSYPPTFGQGTKVEIKRTGGGSGGGGSGGGASEVQLVESGGGLVQPGGSLRLSCAASGFNINSSYIHWVRQAPGKGLEWVAYISPTDGYYRYADSVKGRFTISADTSKNTAYLQMNSLRAEDTAVYYCSRTSDTSYAAMDVWGQGTLVTVSS

SEQ ID NO: 330 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFNISTSYIHWVRQAPGKGLEWVATIDPNDGYSRYADSVKGRFTISADTSKNTAYLQMNSLRAEDTAVYYCSRTNNTAADAMDVWGQGTLVTVSS

SEQ ID NO: 331 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSGYAPITFGQGTKVEIKRTGGGSGGGGSGGGASEVQLVESGGGLVQPGGSLRLSCAASGFNISYYSMHWVRQAPGKGLEWVADVDPDSDYTEYADSVKGRFTISADTSKNTAYLQMNSLRAEDTAVYYCSRSWIHMFSMDYWGQGTLVTVSS

SEQ ID NO: 332 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYAPITFGQGTKVEIKRTGGGSGGGGSGGGASEVQLVESGGGLVQPGGSLRLSCAASGFNIGYYTMHWVRQAPGKGLEWVAEVSPWSYSTSYADSVKGRFTISADTSKNTAYLQMNSLRAEDTAVYYCSRDHWDEAFDVWGQGTLVTVSS

SEQ ID NO: 333 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSLYGPFTFGQGTKVEIKRTGGGSGGGGSGGGASEVQLVESGGGLVQPGGSLRLSCAASGFNIAYEYMHWVRQAPGKGLEWVALIGPDSGYTYYADSVKGRFTISADTSKNTAYLQMNSLRAEDTAVYYCSRVWYYSTYGMDYWGQGTLVTVSS

SEQ ID NO: 334 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYSPITFGQGTKVEIKRTGGGSGGGGSGGGASEVQLVESGGGLVQPGGSLRLSCAASGFNLFGYGMHWVRQAPGKGLEWVAEIGPYYYYTSYADSVKGRFTISADTSKNTAYLQMNSLRAEDTAVYYCSRENYDMAMDYWGQGTLVTVSS

SEQ ID NO: 335 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSGYQPDTFGQGTKVEIKRTGGGSGGGGSGGGASEVQLVESGGGLVQPGGSLRLSCAASGFNISWYAMHWVRQAPGKGLEWVAEIWPDSDWTYYADSVKGRFTISADTSKNTAYLQMNSLRAEDTAVYYCSRYYYSSAFDVWGQGTLVTVSS

SEQ ID NO: 336 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYLYQPWTFGQGTKVEIKRTGGGSGGGGSGGGASEVQLVESGGGLVQPGGSLRLSCAASGFNIDYYGMHWVRQAPGKGLEWVASLYGGSDSTDYADSVKGRFTISADTSKNTAYLQMNSLRAEDTAVYYCSRQYSAYFDYWGQGTLVTVSS

SEQ ID NO: 337 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGLYYPWTFGQGTKVEIKRTGGGSGGGGSGGGASEVQLVESGGGLVQPGGSLRLSCAASGFNVSYSSIHWVRQAPGKGLEWVAEIWPDSGQTWYADSVKGRFTISADTSKNTAYLQMNSLRAEDTAVYYCSRSSYFDAMDYWGQGTLVTVSS

SEQ ID NO: 338 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQWYSSPVTFGQGTKVEIKRTGGGSGGGGSGGGASEVQLVESGGGLVQPGGSLRLSCAASGFNINWANMHWVRQAPGKGLEWVAQISPPYDYTNYADSVKGRFTISADTSKNTAYLQMNSLRAEDTAVYYCSRSYSYYFDYWGQGTLVTVSS

SEQ ID NO: 339 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYSRPVTFGQGTKVEIKRTGGGSGGGGSGGGASEVQLVESGGGLVQPGGSLRLSCAASGFNIYLHDMHWVRQAPGKGLEWVAQIIPAIDYTNYADSVKGRFTISADTSKNTAYLQMNSLRAEDTAVYYCSRRDGYYFDYWGQGTLVTVSS

SEQ ID NO: 340 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQWYSSPVTFGQGTKVEIKRTGGGSGGGGSGGGASEVQLVESGGGLVQPGGSLRLSCAASGFNIYWSHMHWVRQAPGKGLEWVAIISSFEGYTYYADSVKGRFTISADTSKNTAYLQMNSLRAEDTAVYYCSRSYSYYMDYWGQGTLVTVSS

SEQ ID NO: 341 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYGSGSPWTFGQGTKVEIKRTGGGSGGGGSGGGASEVQLVESGGGLVQPGGSLRLSCAASGFNIVGGGIHWVRQAPGKGLEWVAKIYPQGDYTYYADSVKGRFTISADTSKNTAYLQMNSLRAEDTAVYYCSRDSSYLAFDYWGQGTLVTVSS

SEQ ID NO: 342 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQTYYSPWTFGQGTKVEIKRTGGGSGGGGSGGGASEVQLVESGGGLVQPGGSLRLSCAASGFNIRSYAMHWVRQAPGKGLEWVAQVGPGKGYTYYADSVKGRFTISADTSKNTAYLQMNSLRAEDTAVYYCSRNFQSTSHAFDYWGQGTLVTVSS

SEQ ID NO: 343 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYYYPPITFGQGTKVEIKRTGGGSGGGGSGGGASEVQLVESGGGLVQPGGSLRLSCAASGFNVSHTGMHWVRQAPGKGLEWVAVVGPGKGYTYYADSVKGRFTISADTSKNTAYLQMNSLRAEDTAVYYCSRKTYYAFDYWGQGTLVTVSS

SEQ ID NO: 344 - protein, 245 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYYFRPITFGQGTKVEIKRTGGGSGGGGSGGGASEVQLVESGGGLVQPGGSLRLSCAASGFNLSYSDIHWVRQAPGKGLEWVAVVMPDSGHTNYADSVKGRFTISADTSKNTAYLQMNSLRAEDTAVYYCSRATNIPVYAFDYWGQGTLVTVSS

SEQ ID NO: 345 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQASYYYPLTFGQGTKVEIKRTGGGSGGGGSGGGASEVQLVESGGGLVQPGGSLRLSCAASGFNISASGMHWVRQAPGKGLEWVADIHPLKPYTNYADSVKGRFTISADTSKNTAYLQMNSLRAEDTAVYYCSRYSSMYYYYFDYWGQGTLVTVSS

SEQ ID NO: 346 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQKSEYSPWTFGQGTKVEIKRTGGGSGGGGSGGGASEVQLVESGGGLVQPGGSLRLSCAASGFNIYRYGIHWVRQAPGKGLEWVAVLYPYGYSTSYADSVKGRFTISADTSKNTAYLQMNSLRAEDTAVYYCSRSYAYGYFAYWGQGTLVTVSS

SEQ ID NO: 347 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSGYIPFTFGQGTKVEIKRTGGGSGGGGSGGGASEVQLVESGGGLVQPGGSLRLSCAASGFNIYGTMMHWVRQAPGKGLEWVAQFKPDSYNTYYADSVKGRFTISADTSKNTAYLQMNSLRAEDTAVYYCSRGEVYHYYAFDYWGQGTLVTVSS

SEQ ID NO: 348 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGAYYRPFTFGQGTKVEIKRTGGGSGGGGSGGGASEVQLVESGGGLVQPGGSLRLSCAASGFNISYSYMHWVRQAPGKGLEWVATLLPYDGNTYYADSVKGRFTISADTSKNTAYLQMNSLRAEDTAVYYCSRAAYSSMDVWGQGTLVTVSS

SEQ ID NO: 349 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYMYSPVTFGQGTKVEIKRTGGGSGGGGSGGGASEVQLVESGGGLVQPGGSLRLSCAASGFNVSAYWMHWVRQAPGKGLEWVAQIYGGSGYTMYADSVKGRFTISADTSKNTAYLQMNSLRAEDTAVYYCSRTHSYWSAFDYWGQGTLVTVSS

SEQ ID NO: 350 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSSSPITFGQGTKVEIKRTGGGSGGGGSGGGASEVQLVESGGGLVQPGGSLRLSCAASGFNISGYGMHWVRQAPGKGLEWVAYLYGGSDYTNYADSVKGRFTISADTSKNTAYLQMNSLRAEDTAVYYCSRTVRYAFDYWGQGTLVTVSS

SEQ ID NO: 351 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSASPLTFGQGTKVEIKRTGGGSGGGGSGGGASEVQLVESGGGLVQPGGSLRLSCAASGFNVSSVGMHWVRQAPGKGLEWVAYIYGTSDYTYYADSVKGRFTISADTSKNTAYLQMNSLRAEDTAVYYCSRSSRYSMDYWGQGTLVTVSS

SEQ ID NO: 352 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYAYSPLTFGQGTKVEIKRTGGGSGGGGSGGGASEVQLVESGGGLVQPGGSLRLSCAASGFNVSSYGMHWVRQAPGKGLEWVAFIAPRRDYTSYADSVKGRFTISADTSKNTAYLQMNSLRAEDTAVYYCSRKSSYYFDYWGQGTLVTVSS

SEQ ID NO: 353 - protein, 246 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYYPITFGQGTKVEIKRTGGGSGGGGSGGGASEVQLVESGGGLVQPGGSLRLSCAASGFNFSYGYMHWVRQAPGKGLEWVAWISGYTGNTYYADSVKGRFTISADTSKNTAYLQMNSLRAEDTAVYYCSRAASLSSSYYSAFDVWGQGTLVTVSS

SEQ ID NO: 354 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYTPVTFGQGTKVEIKRTGGGSGGGGSGGGASEVQLVESGGGLVQPGGSLRLSCAASGFNVWGPGMHWVRQAPGKGLEWVARIHPFSGNTYYADSVKGRFTISADTSKNTAYLQMNSLRAEDTAVYYCSRGYSYSAMDYWGQGTLVTVSS

SEQ ID NO: 355 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYEPVTFGQGTKVEIKRTGGGSGGGGSGGGASEVQLVESGGGLVQPGGSLRLSCAASGFNVSGSQMHWVRQAPGKGLEWVARIPGWSGYTYYADSVKGRFTISADTSKNTAYLQMNSLRAEDTAVYYCSRGYSYFAMDYWGQGTLVTVSS

SEQ ID NO: 356 - protein, 245 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYAYYSPVTFGQGTKVEIKRTGGGSGGGGSGGGASEVQLVESGGGLVQPGGSLRLSCAASGFNIYGQMMHWVRQAPGKGLEWVAFLSPFSGNTYYADSVKGRFTISADTSKNTAYLQMNSLRAEDTAVYYCSRNISYEQSSAFDYWGQGTLVTVSS

SEQ ID NO: 357 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYEYYPMTFGQGTKVEIKRTGGGSGGGGSGGGASEVQLVESGGGLVQPGGSLRLSCAASGFNVMYSTMHWVRQAPGKGLEWVASIYSWSDYTYYADSVKGRFTISADTSKNTAYLQMNSLRAEDTAVYYCSRGYAHNSFDYWGQGTLVTVSS

SEQ ID NO: 358 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSFYPFTFGQGTKVEIKRTGGGSGGGGSGGGASEVQLVESGGGLVQPGGSLRLSCAASGFNFGSYIHWVRQAPGKGLEWVAIISGYSGNTYYADSVKGRFTISADTSKNTAYLQMNSLRAEDTAVYYCSRSNQSAYSYMDYWGQGTLVTVSS

SEQ ID NO: 359 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYSPITFGQGTKVEIKRTGGGSGGGGSGGGASEVQLVESGGGLVQPGGSLRLSCAASGFNISDSYMHWVRQAPGKGLEWVATFSPYSSNTWYADSVKGRFTISADTSKNTAYLQMNSLRAEDTAVYYCSRSQFTFYQYFDYWGQGTLVTVSS

SEQ ID NO: 360 - protein, 244 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQYSAYYQPITFGQGTKVEIKRTGGGSGGGGSGGGASEVQLVESGGGLVQPGGSLRLSCAASGFNIFSDQMHWVRQAPGKGLEWVAGFMPYDSYYTNYADSVKGRFTISADTSKNTAYLQMNSLRAEDTAVYYCSRMSVRNAFDYWGQGTLVTVSS

SEQ ID NO: 361 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYYPITFGQGTKVEIKRTGGGSGGGGSGGGASEVQLVESGGGLVQPGGSLRLSCAASGFNLSYSYMHWVRQAPGKGLEWVAVISGFSGNTYYADSVKGRFTISADTSKNTAYLQMNSLRAEDTAVYYCSRSDSYYTAMDYWGQGTLVTVSS

SEQ ID NO: 362 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYEYVPHTFGQGTKVEIKRTGGGSGGGGSGGGASEVQLVESGGGLVQPGGSLRLSCAASGFNISYGYMHWVRQAPGKGLEWVAKFHYGSGNTYYADSVKGRFTISADTSKNTAYLQMNSLRAEDTAVYYCSRSNYYYLDYWGQGTLVTVSS

SEQ ID NO: 363 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYMPITFGQGTKVEIKRTGGGSGGGGSGGGASEVQLVESGGGLVQPGGSLRLSCAASGFNISYQHIHWVRQAPGKGLEWVAVFMPYQGSTYYADSVKGRFTISADTSKNTAYLQMNSLRAEDTAVYYCSRANIYSSHSFFDYWGQGTLVTVSS

SEQ ID NO: 364 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYAYYPVTFGQGTKVEIKRTGGGSGGGGSGGGASEVQLVESGGGLVQPGGSLRLSCAASGFNLSGYYMHWVRQAPGKGLEWVAWFSPYSGYTYYADSVKGRFTISADTSKNTAYLQMNSLRAEDTAVYYCSRTHSSIYHSFDYWGQGTLVTVSS

SEQ ID NO: 365 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYMPITFGQGTKVEIKRTGGGSGGGGSGGGASEVQLVESGGGLVQPGGSLRLSCAASGFNVSGQYMHWVRQAPGKGLEWVAVISPVSGNTYYADSVKGRFTISADTSKNTAYLQMNSLRAEDTAVYYCSRPMKTSYYGAFDYWGQGTLVTVSS

SEQ ID NO: 366 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDYRPVTFGQGTKVEIKRTGGGSGGGGSGGGASEVQLVESGGGLVQPGGSLRLSCAASGFNVSTSGMHWVRQAPGKGLEWVAFIYGAYSGTYYADSVKGRFTISADTSKNTAYLQMNSLRAEDTAVYYCSRSQSYTYWSAMDYWGQGTLVTVSS

SEQ ID NO: 367 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYDFTPMTFGQGTKVEIKRTGGGSGGGGSGGGASEVQLVESGGGLVQPGGSLRLSCAASGFNISYAKMHWVRQAPGKGLEWVAYLTYWGGYTNYADSVKGRFTISADTSKNTAYLQMNSLRAEDTAVYYCSRGEYGTYMDYWGQGTLVTVSS

SEQ ID NO: 368 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSSSSPVTFGQGTKVEIKRTGGGSGGGGSGGGASEVQLVESGGGLVQPGGSLRLSCAASGFNFSSYVMHWVRQAPGKGLEWVAVVYPDSGGTYYADSVKGRFTISADTSKNTAYLQMNSLRAEDTAVYYCSRTSSYYAFDYWGQGTLVTVSS

SEQ ID NO: 369 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSYTPITFGQGTKVEIKRTGGGSGGGGSGGGASEVQLVESGGGLVQPGGSLRLSCAASGFNISQGGIHWVRQAPGKGLEWVAYVYPGGGQTNYADSVKGRFTISADTSKNTAYLQMNSLRAEDTAVYYCSRGYDYSAFDYWGQGTLVTVSS

SEQ ID NO: 370 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYAYYPITFGQGTKVEIKRTGGGSGGGGSGGGASEVQLVESGGGLVQPGGSLRLSCAASGFNISSTGMHWVRQAPGKGLEWVAELLGGSGNTNYADSVKGRFTISADTSKNTAYLQMNSLRAEDTAVYYCSRGLQYSAMDYWGQGTLVTVSS

SEQ ID NO: 371 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYEYYPITFGQGTKVEIKRTGGGSGGGGSGGGASEVQLVESGGGLVQPGGSLRLSCAASGFNFFSTVIHWVRQAPGKGLEWVAEIYPWSGSTYYADSVKGRFTISADTSKNTAYLQMNSLRAEDTAVYYCSRSRSSNYYFDVWGQGTLVTVSS

SEQ ID NO: 372 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYTYYPITFGQGTKVEIKRTGGGSGGGGSGGGASEVQLVESGGGLVQPGGSLRLSCAASGFNLHGYLMHWVRQAPGKGLEWVAFIYPPNGYTYYADSVKGRFTISADTSKNTAYLQMNSLRAEDTAVYYCSRGVDYAYLDYWGQGTLVTVSS

SEQ ID NO: 373 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYSYYPITFGQGTKVEIKRTGGGSGGGGSGGGASEVQLVESGGGLVQPGGSLRLSCAASGFNLSTHVMHWVRQAPGKGLEWVAEFYPYVGYTYYADSVKGRFTISADTSKNTAYLQMNSLRAEDTAVYYCSRGYRYQYMDVWGQGTLVTVSS

SEQ ID NO: 374 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSSVEPWTFGQGTKVEIKRTGGGSGGGGSGGGASEVQLVESGGGLVQPGGSLRLSCAASGFNVSYYSIHWVRQAPGKGLEWVAYIYPWNDYTYYADSVKGRFTISADTSKNTAYLQMNSLRAEDTAVYYCSRGSYYSFDYWGQGTLVTVSS

SEQ ID NO: 375 - protein, 238 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYYSPPTFGQGTKVEIKRTGGGSGGGASEVQLVESGGGLVQPGGSLRLSCAASGFNINNTYIHWVRQAPGKGLEWVASIYPTDGYTRYADSVKGRFTISADTSKNTAYLQMNSLRAEDTAVYYCSRTYYSYYSAMDVWGQGTLVTVSS

SEQ ID NO: 376 - protein, 241 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQIYTSPITFGQGTKVEIKRTGGGSGGGGSGGGASEVQLVESGGGLVQPGGSLRLSCAASGFNFITTGMHWVRQAPGKGLEWVARIGPGSDYTNYADSVKGRFTISADTSKNTAYLQMNSLRAEDTAVYYCSRYYYASALDYWGQGTLVTVSS

SEQ ID NO: 377 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQRAYFPITFGQGTKVEIKRTGGGSGGGGSGGGASEVQLVESGGGLVQPGGSLRLSCAASGFNFSDYGMHWVRQAPGKGLEWVAMLIPASGYTNYADSVKGRFTISADTSKNTAYLQMNSLRAEDTAVYYCSRGWSYYMDYWGQGTLVTVSS

SEQ ID NO: 378 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQQYAYTPITFGQGTKVEIKRTGGGSGGGGSGGGASEVQLVESGGGLVQPGGSLRLSCAASGFNVWSYGIHWVRQAPGKGLEWVAGVTPDGSYTYYADSVKGRFTISADTSKNTAYLQMNSLRAEDTAVYYCSRSYGWAMDYWGQGTLVTVSS

SEQ ID NO: 379 - protein, 241 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQIHYKPLTFGQGTKVEIKRTGGGSGGGGSGGGASEVQLVESGGGLVQPGGSLRLSCAASGFNVAWYSIHWVRQAPGKGLEWVAQVYGGSSYTYYADSVKGRFTISADTSKNTAYLQMNSLRAEDTAVYYCSRDFYSSGMDYWGQGTLVTVSS

SEQ ID NO: 380 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQEWRLPITFGQGTKVEIKRTGGGSGGGGSGGGASEVQLVESGGGLVQPGGSLRLSCAASGFNVYGNQIHWVRQAPGKGLEWVARIYPYSGSTYYADSVKGRFTISADTSKNTAYLQMNSLRAEDTAVYYCSRSAYVAYSYFDYWGQGTLVTVSS

SEQ ID NO: 381-389 - skipped (9 entries)

SEQ ID NO: 390 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGTSTPFTFGQGTKVEIKRTGGGSGGGGSGGGASEVQLVESGGGLVQPGGSLRLSCAASGFNLSYYGMHWVRQAPGKGLEWVATIYPDSGYTKYADSVKGRFTISADTSKNTAYLQMNSLRAEDTAVYYCSRAYLYYYLAYWGQGTLVTVSS

SEQ ID NO: 391 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSWRYPMTFGQGTKVEIKRTGGGSGGGGSGGGASEVQLVESGGGLVQPGGSLRLSCAASGFNISRYGMHWVRQAPGKGLEWVAVFYPSSSYTYYADSVKGRFTISADTSKNTAYLQMNSLRAEDTAVYYCSRKYYEAMDYWGQGTLVTVSS

SEQ ID NO: 392 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYSYPVTFGHGTKVEIKRTGGGSGGGGSGGGASEVQLVESGGGLVQPGGSLRLSCAASGFNIYSSWMHWVRQAPGKGLEWVAYFQPYSGYTKYADSVKGRFTISADTSKNTAYLQMNSLRAEDTAVYYCSREYTYYFDYWGQGTLVTVSS

SEQ ID NO: 393 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGWLYSPFTFGQGTKVEIKRTGGGSGGGGSGGGASEVQLVESGGGLVQPGGSLRLSCAASGFNISGYGMHWVRQAPGKGLEWVARVYGGSGYTYYADSVKGRFTISADTSKNTAYLQMNSLRAEDTAVYYCSRAHSSYYVDYWGQGTLVTVSS

SEQ ID NO: 394 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGYFYYPNTFGQGTKVEIKRTGGGSGGGGSGGGASEVQLVESGGGLVQPGGSLRLSCAASGFNIGYYGMHWVRQAPGKGLEWVATVYPGGGYTSYADSVKGRFTISADTSKNTAYLQMNSLRAEDTAVYYCSRYYYYGFDYWGQGTLVTVSS

SEQ ID NO: 395 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYYSPVTFGQGTKVEIKRTGGGSGGGGSGGGASEVQLVESGGGLVQPGGSLRLSCAASGFNIFYQDMHWVRQAPGKGLEWVAMIYPDYDYTYYADSVKGRFTISADTSKNTAYLQMNSLRAEDTAVYYCSRTYSVYMDYWGQGTLVTVSS

SEQ ID NO: 396 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYAYAPFTFGQGTKVEIKRTGGGSGGGGSGGGASEVQLVESGGGLVQPGGSLRLSCAASGFNVSYSMIHWVRQAPGKGLEWVARVWGDGGVTYYADSVKGRFTISADTSKNTAYLQMNSLRAEDTAVYYCSRGSYYAFDYWGQGTLVTVSS

SEQ ID NO: 397 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQAHMIPITFGQGTKVEIKRTGGGSGGGGSGGGASEVQLVESGGGLVQPGGSLRLSCAASGFNFSFPGMHWVRQAPGKGLEWVAWFVGYDGYTYYADSVKGRFTISADTSKNTAYLQMNSLRAEDTAVYYCSRDYYSFSMDYWGQGTLVTVSS

SEQ ID NO: 398 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSVYDPITFGQGTKVEIKRTGGGSGGGGSGGGASEVQLVESGGGLVQPGGSLRLSCAASGFNISGSWIHWVRQAPGKGLEWVAWLYPDSDYTYYADSVKGRFTISADTSKNTAYLQMNSLRAEDTAVYYCSRAHTYAFDYWGQGTLVTVSS

SEQ ID NO: 399 - protein, 249 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQSYTSPLTFGQGTKVEIKRTGGGSGGGGSGGGASEVQLVESGGGLVQPGGSLRLSCAASGFNIYYGVMHWVRQAPGKGLEWVAMIYPDSSWTYYADSVKGRFTISADTSKNTAYLQMNSLRAEDTAVYYCSRDQDFHYMNYYLSYALDYWGQGTLVTVSS

SEQ ID NO: 400 - protein, 240 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQGQYSPFTFGQGTKVEIKRTGGGSGGGGSGGGASEVQLVESGGGLVQPGGSLRLSCAASGFNVSYEYMHWVRQAPGKGLEWVAEIYGGSDNTYYADSVKGRFTISADTSKNTAYLQMNSLRAEDTAVYYCSRPLGSYFDYWGQGTLVTVSS

SEQ ID NO: 401 - protein, 241 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQYWYLPTTFGQGTKVEIKRTGGGSGGGGSGGGASEVQLVESGGGLVQPGGSLRLSCAASGFNISWYDIHWVRQAPGKGLEWVADIEPSVGYTYYADSVKGRFTISADTSKNTAYLQMNSLRAEDTAVYYCSRSYPYYYFDYWGQGTLVTVSS

SEQ ID NO: 402 - protein, 8 aa, synthetic construct
GFNLSYYG

SEQ ID NO: 403 - protein, 10 aa, synthetic construct
ELAGIGILTV

SEQ ID NO: 404 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 405 - protein, 244 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 406 - protein, 240 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 407 - protein, 249 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 408 - protein, 240 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 409 - protein, 240 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 410 - protein, 249 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 411 - protein, 241 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSS

SEQ ID NO: 412 - protein, 241 aa, synthetic construct
EIVLTQSPRTLSLSPGERATLSCRASQSVSSSYLAWYQQKPGQAPRLLIYGASSRATGIPDRFSGSGSGTDFTLTISRLDPEDFAVYYCQQYGSSPITFGQGTRLEIKGGGGSGGGGSGGGGSQVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 413 - protein, 239 aa, synthetic construct
DILMTQSPSSLSASVGDRVTITCRASQGISSALAWYQQKPGKAPKLLIYYASSLQSGVPSRFSGSGSGTDYTLTISSLQPEDFATYYCQQYYSTLTFGGGTKVEIKGGGGSGGGGSGGGGSQVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 414 - protein, 241 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSQVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 415 - protein, 240 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 416 - protein, 240 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 417 - protein, 241 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETKRGGGGSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSS

SEQ ID NO: 418 - protein, 10 aa, synthetic construct
SPGAANKRPI

SEQ ID NO: 419 - protein, 9 aa, synthetic construct
VVGACGVGK

SEQ ID NO: 420 - protein, 9 aa, synthetic construct
VVGADGVGK